(19) United States
(12) Patent Application Publication
(10) Pub. No.: US 2022/0356529 A1
(43) Pub. Date: Nov. 10, 2022

(54) METHOD OF ASSESSING THE SENSITIVITY OR RESISTANCE OF A SUBJECT TO AN ONCOLYTIC VIRUS, RECOMBINANT VIRUS, PREPARATION AND USES THEREOF

(71) Applicants: UNIVERSITÉ COTE D'AZUR, NICE CEDEX 2 (FR); INSTITUT NATIONAL DE LA SANTE ET DE LA RECHERCHE MEDICALE, PARIS (FR); COMMISSARIAT A L'ENERGIE ATOMIQUE ET AUX ENERGIES ALTERNATIVES, PARIS (FR)

(72) Inventors: BÉATRICE CAMBIEN, CHATEAUNEUF-VILLEVIEILLE (FR); GEORGES VASSAUX, PEGOMAS (FR); ROBERT BARTHEL, CHATEAUNEUF-VILLEVIEILLE (FR)

(21) Appl. No.: 17/625,791

(22) PCT Filed: Jul. 16, 2020
(86) PCT No.: PCT/EP2020/070095

§ 371 Date: Jan. 10, 2022

(30) Foreign Application Priority Data
Jul. 16, 2019 (EP) 19315072.9

(51) Int. Cl. C12Q 1/6886

(52) U.S. Cl. CPC C12Q 1/6886; C12Q 2600/106; C12Q 2600/158

(57) ABSTRACT

The present invention relates to the field of medicine and in particular to the treatment of cancer. More particularly, the invention relates to a method of assessing the sensitivity or resistance of a subject having a cancer to an oncolytic virus, to a method of selecting a treatment comprising an oncolytic virus efficient against the cancer of a subject and to a method of monitoring in a subject the response to a cancer treatment comprising an oncolytic virus. The description further relates to products including a therapeutic recombinant virus, in particular a recombinant oncolytic virus, typically a vaccinia virus, a pharmaceutical composition, and a kit comprising such a therapeutic virus, as well as preparation and uses thereof.

### FIELD OF THE INVENTION

The present invention relates to the field of medicine and in particular to the treatment of cancer. More particularly, the invention relates to a method of assessing the sensitivity or resistance of a subject having a cancer to an oncolytic virus, to a method of selecting a treatment comprising an oncolytic virus efficient against the cancer of a subject and to a method of monitoring in a subject the response to a cancer treatment comprising an oncolytic virus, and, if required, of stopping or adapting the treatment.

The description further relates to products including a therapeutic recombinant virus, in particular a recombinant oncolytic virus, typically a vaccinia virus, a pharmaceutical composition, and a kit comprising such a therapeutic virus, as well as preparation and uses thereof.

### BACKGROUND OF THE INVENTION

Oncolytic vaccinia viruses (VV) represent a new class of anticancer agents with multiple mechanisms of action. VV have been shown to act at three distinct levels (Kirn and Thorne, 2009).

VV infects and selectively replicates in cancer cells, leading to primary oncolysis and resulting in cancer cell destruction (Heise and Kirn, 2000). They also disrupt the tumor vasculature (Breitbach et al., 2013), and reduce tumor perfusion. Finally, the release of tumor antigens from dead tumor cells participates to the initiation of an immune response that will be effective against tumor cells (Achard et al., 2018; Breitbach et al., 2015; Kirn and Thorne, 2009; Thorne, 2011). In humans, VV, administered intratumorally or systemically has been well-tolerated in various clinical trials (Breitbach et al., 2015).

Poxviruses are large viruses with cytoplasmic sites of replication and they are considered less dependent on host cell functions than other DNA viruses. Nevertheless, the existence of cellular proteins capable of inhibiting or enhancing poxvirus replication and spread has been demonstrated. Cellular proteins such as dual specific phosphatase 1 DUSP1 (Caceres et al., 2013) or barrier to autointegration factor (BAF) (Ibrahim et al., 2011) have been shown to be detrimental to the virus. On the opposite the ubiquitin ligase cullin-3 has been shown to be required for the initiation of viral DNA replication (Mercer et al., 2012). Furthermore, high-throughput RNA interference screens have suggested the potential role of hundreds of proteins acting as either restricting or promoting factors for VV (Beard et al., 2014; Mercer et al., 2012; Sivan et al., 2013; Sivan et al., 2015). These studies highlight the importance of cellular factors in VV replication and spread. The concept of resistance to primary oncolysis to VV has, so far, never been formally demonstrated. For example, in the field of breast cancer research, in vitro testing in established human cell lines and in vivo xenografts in mice, have shown clearly and convincingly that VV has anti-tumor activity against breast cancer (Gholami et al., 2012; Zhang et al., 2007). The efficacy of VV was evident in triple-negative high-grade breast carcinoma mouse models (Gholami et al., 2012), a pathology associated with poor prognostic and for which new therapeutic options are urgently needed. Nevertheless, these studies were performed in established cancer cell lines that may differ from the actual carcinoma cells present in the tumors.

Spontaneously occurring mammary cancers in dogs are of potential interest in the development of new anticancer agents (Khanna, 2017; Paoloni and Khanna, 2008) as, as a whole, the classification of canine breast carcinoma is relevant to that of human's (Gama et al., 2008; Pinho et al., 2012; Sassi et al., 2010). If differences have been highlighted in complex carcinomas (Liu et al., 2014), simple canine carcinomas faithfully represent human breast carcinomas, both at the histological and molecular level (Gama et al., 2008; Sassi et al., 2010). This is particularly the case for the so called “triple negative carcinomas” (wherein a lack of estrogen and progesterone receptors and of epidermal growth factor receptor type 2 are observed) (Jaillardon et al., 2015; Kim et al., 2013) for which therapeutic options are limited and unsatisfactory.

Inventors herein demonstrate, with vaccinia viruses, that cancer cells are not equally sensitive to oncolytic viruses. This observation is in sharp contrast with the fact the same virus is considered by the skilled person to be equally efficient in established cell lines (Cree et al., 2010) for example from differentiated/low-grade and in high-grade triple negative carcinoma cells (TNBC). They herein advantageously describe a method of assessing the sensitivity or resistance of a subject having a cancer to an oncolytic virus, a method of monitoring in a subject the response to a cancer treatment comprising an oncolytic virus, and, if required, of stopping or adapting the treatment, and a method of selecting a treatment comprising an oncolytic virus efficient against the cancer of a subject, as well as a new recombinant oncolytic virus and compositions and kits comprising such a recombinant virus.

Other advantages of the methods, products and compositions herein described are further indicated below.

### SUMMARY OF THE INVENTION

Inventors herein describe methods, typically in vitro or ex vivo methods, of assessing the sensitivity or resistance of a subject having a cancer to an oncolytic virus.

A particular method comprises a step of determining, in a biological sample from a subject, the expression or, on the contrary, lack of expression of at least one gene of interest, typically the presence or absence of a protein/mRNA encoded by a gene selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, thereby assessing whether the subject having a cancer is sensitive or resistant to the oncolytic virus.

Another particular method comprises a step a) of determining, in a biological sample from a subject, the presence or absence of, and if present the expression level of, and/or percentage of cells expressing, a protein/mRNA encoded by a gene selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, and, when the expression level of, and/or percentage of cells expressing, the protein/mRNA is determined, a step b) of comparing said expression level to a reference expression level and/or said percentage of cells to a reference percentage of cells, thereby assessing whether the subject having a cancer is sensitive or resistant to the oncolytic virus.

Also provided is a method, typically an in vitro or ex vivo a method, of monitoring in a subject the response to a cancer treatment comprising an oncolytic virus, and if required of stopping or adapting the treatment. This method comprises a step a) of determining at a first time point, T0, the expression level of (herein identified as “protein/mRNA reference expression level”), and/or the percentage of cells expressing (herein identified as “reference percentage of cells”), a protein/mRNA encoded by a gene selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in a biological sample of a subject having a cancer, before any step of cancer treatment applied to the subject or at about the same time as, typically when, beginning a cancer treatment in the subject, or after a step of cancer treatment applied to the subject, a step a′) of determining in a biological sample of the subject having a cancer obtained at a different time point, for example T1, T1 following T0, the protein/mRNA response expression level and/or percentage of responding cells expressing the protein, after the administration to said subject of a first, or additional, therapeutic dose of an oncolytic virus for treating the cancer, and a step b) of comparing said protein/mRNA response expression level to said protein/mRNA reference expression level and/or to a protein/mRNA reference expression level in a control population, and/or of comparing said percentage of responding cells expressing the protein to said reference percentage of cells and/or to a reference percentage of cells in a control population, a protein/mRNA response expression level identical to or below the protein/mRNA reference expression level(s), and/or a percentage of responding cells expressing the protein identical to or below the reference percentage of cells, being the indication that the oncolytic virus will be efficient as such against the cancer of the subject, whereas a protein/mRNA response expression level above the protein/mRNA reference expression level(s), and/or a percentage of responding cells expressing the protein above the reference percentage of cells, being the indication that an oncolytic virus will not be efficient alone in the subject, and if the oncolytic virus is not efficient as such, a step c) of stopping or adapting the treatment of the subject's cancer, for example by selecting a treatment combining said oncolytic virus with an additional compound, in particular an additional protein selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, or a treatment comprising a therapeutic recombinant oncolytic virus.

Inventors in addition herein describe a method, typically an in vitro or ex vivo method, of selecting a treatment comprising an oncolytic virus efficient against the cancer of a subject. This method comprises, typically in the following order:
a step a) of determining the presence or absence of, and preferably, if present, the basal expression level of, or basal percentage of cells expressing, a protein/mRNA encoded by a gene selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in a biological sample of a subject having a cancer, before any step of cancer treatment, in particular of cancer treatment comprising an oncolytic virus, applied to the subject, or at about the same time as, typically when, beginning such a cancer treatment in the subject,
a step a′) of determining, in a biological sample of the subject having a cancer, the protein/mRNA response expression level, or percentage of cells expressing the protein, after the administration to said subject of at least one therapeutic dose of an oncolytic virus for treating the cancer,
a step b) of:
   comparing said protein/mRNA response expression level to said protein/mRNA basal expression level and/or to a protein/mRNA reference expression level in a control population, and/or of
   comparing said percentage of cells expressing the protein to said basal percentage of cells and/or to a reference percentage of cells in a control population, and
a step c) of selecting an appropriate treatment of the subject's cancer, wherein:
   a protein/mRNA response expression level identical to or below the protein/mRNA basal and/or reference expression level, and/or a percentage of cells expressing the protein identical to or below the basal and/or reference percentage of cells, is the indication that the oncolytic virus will be efficient as such and is to be selected for treating the cancer of the subject, whereas
   a protein/mRNA response expression level above the protein/mRNA basal and/or reference expression level, and/or a percentage of cells expressing the protein above the basal and/or reference percentage of cells, is the indication that the oncolytic virus will not be efficient as such, and that an appropriate treatment of the subject's cancer is to be selected, the appropriate treatment being (consisting in) for example a treatment combining said oncolytic virus with an additional compound, in particular an additional protein selected typically from DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, or a treatment comprising a therapeutic recombinant oncolytic virus.

Inventors also herein advantageously describe a therapeutic recombinant virus, preferably a therapeutic recombinant oncolytic virus. This therapeutic recombinant virus comprises a nucleic acid for modulating a gene or its expression product in a cell, the gene being selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, and being preferably DDIT4. When the desired modulation is an inhibition, the nucleic acid is preferably selected from a si-RNA, a sh-RNA, an antisense-DNA, an antisense-RNA and a ribozyme.

In a particular aspect, the therapeutic recombinant virus comprises a nucleic acid for inhibiting DDIT4 or its expression product in a cell, and the nucleic acid is selected from a si-RNA, a sh-RNA, an antisense-DNA, an antisense-RNA and a ribozyme.

Herein described is also a pharmaceutical composition comprising a therapeutic recombinant virus as herein described and pharmaceutically acceptable carrier(s) and/or excipient(s).

Inventors also herein describe a therapeutic recombinant virus or pharmaceutical composition as herein described for use as a medicament or for preparing such a medicament, in particular for use in the prevention or treatment of a cancer.

A kit comprising at least one antibody used as a detection means, this antibody being specific to a protein; a molecule allowing the antibody detection; and, optionally, a leaflet providing the protein reference expression level, and/or the reference percentage of cells expressing the protein, in control population(s), and/or a therapeutic recombinant virus, is also described, as well as the use of such a kit for assessing the sensitivity or resistance of a subject having a cancer to an oncolytic virus, or for monitoring in a subject the response to a cancer treatment comprising an oncolytic virus, and, optionally, for preventing or treating the cancer of the subject.

The detection means of the kit is preferably selected from the group consisting of at least one antibody specific to DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 or TBCB, and the leaflet provides the DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and/or TBCB, respective reference expression level(s), and/or reference percentage(s) of cells expressing a protein selected from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in control population(s).

### LEGEND TO THE FIGURES

FIG. 1: Triple-negative breast carcinoma (TNBC) cells are resistant to oncolytic VV-tk⁻.

A. Non-TNBC (white bars) or TNBC (black bars) cells were infected at different multiplicities of infection with VV-tk⁻. Four days later the number of cells in the wells were counted and the EC50 (dose of virus capable of killing 50% of the cell population) was calculated. Data show the mean EC50+/−SE obtained on twenty independent non-TNBC and TNBC cultures.

B. Twenty-four hours after VV-tk⁻ infection, non-TNBC (white bars) or TNBC (black bars) cells were collected. DNA was isolated and subjected to quantitative PCR to titer the number of viral genome per well.

C. Three days after VV-tk⁻ infection, non-TNBC (white bars) or TNBC (black bars) cells were collected, homogenized and the number of infectious particles generated in the culture dishes were titrated on HeLa cells.

D. Non-TNBC (white bars) or TNBC (black bars) cells were infected with a VV-tk⁻ in which the expression of the GFP is driven by an immediate-early VV-tk⁻ promoter. Two hours after infection, the cells were fixed and stained with propidium iodide (PI). The number of PI and GFP positive was determined and the ratio GFP+/PI+ was calculated and is presented.

E and F. Non-TNBC (white bars) or TNBC (black bars) cells were infected with a VV-tk⁻. Six hours after infection, the cells were fixed and stained with PI. The number of cells with mini-nuclei (E) as well as the number of mini-nuclei in mini-nuclei-positive cells (F) were determined. (***: p<0.001; ** p<0.01; * p<0.05).

FIG. 2: Comparison of the viral gene transcription in non-TNBC and TNBC carcinoma cells infected with VV-tk. TNBC or non-TNBC carcinoma cells were infected at a multiplicity of infection of 5. At different times post-infection (2, 4, 8 hours), the cells were collected and processed for RNA sequencing. The data represent the levels of early (A), intermediate (B) or late (C) viral gene expression.

FIG. 3: Characterization of TNBC cells infection by VV-tk⁻ using single-cell transcriptomic analysis.

A, B: Two independent TNBC primary cell cultures (A and B) were either mock infected or infected with VV-tk⁻ at a MOI of 5. Six hours later, the cells were trypsinized and subjected to the 10× Genomics single-cell protocol, followed by NG sequencing. The number of cellular gene expressed decreases as the extent of viral gene expression increases.

C, D: a t-SNE plot was created using the dataset obtained with the naïve cells (uninfected) and after exclusion of the cycling cells: plots were segregated in clusters.

E, F: Three distinct cellular populations were defined: naïve cells, defined as cells not exposed to VV-tk⁻; bystander cells, defined as cells exposed to the virus but expressing less than 1% of early viral genes; infected cells, defined as expressing more than 1% of early viral genes. Naïve, bystander and infected cells were localized onto the t-SNE plot.

FIG. 4: A, B: Top 20 genes differentially expressed in the infected cells versus bystander cells using a differential transcriptional analysis on two cellular population TNBC A and B. C: Six differentially expressed genes are commons to the two experiments.

FIG. 5: A to E: Top 20 genes differentially expressed in the infected cells versus bystander cells using a analysis in individual clusters (in the two primary TNBC populations). F: Top 15 genes overrepresented in the five clusters providing a list of genes for which the differential expression was statistically significant.

FIG. 6: Comparison of the bulk analysis and the cluster analysis provided a total of 15 genes (A) as 5 of the 6 genes selected in the aggregated cluster analysis (B) were also present in the list of selected genes in the “by cluster” analysis.

FIG. 7: Role of DDIT4 in VV-tk⁻ replication. Production of infectious VV particles after infection of (A) HeLa cells overexpressing DDIT4 compared to control HeLa cells expressing GFP, (B) mouse embryonic fibroblast (MEF) from DDIT4 knock out mice compared to MEF isolated from wild-type mice. (C) DDIT4 expression assessed by western blotting in parental HeLa cells, either untreated or treated with increasing concentrations of CoCl2 or with VV.

FIG. 8: Cells from triple negative carcinomas cells (TNBC) or non-TNBC cells were infected with VV-tk⁻ at different multiplicity of infection (MOI) and the numbers of cells remaining in the culture wells were monitored after four days. FIG. 8A presents an example of dose-response curves showing that non-TNBC cells are more sensitive to VV-tk⁻-mediated cell lysis than TNBC cells. FIG. 8B presents an example of dose-response curves showing that in human established cell lines, MCF7 cells (as a representative of the non-TNBC cells) and MDA-MB-231 cells (as a representative of TNBC cells) show an equivalent sensitivity to VV-tk⁻-induced cell lysis.

FIG. 9: Efficacy of infection and replication of VV-tk⁻ within grade 2 and grade 3 mammary carcinoma cells. Eight hours after infection by VV-tk-GFP, the intracellular presence of the virus is visualized in green within carcinoma cells from both grades whereas mininuclei, indicating viral replication, are barely detectable within grade 3 cells.

FIG. 10: Single-cell transcriptomic analysis of TNBC infected with VV (experiment 1 or example 2). Cells from TNBC were either mock infected or infected with VV (MOI=5). Six hours later the cells were subjected to the 10× Genomics single-cell protocol, followed by sequencing. According to the number of clusters to be identified, colored dots were used.

A: UMAP representing the three clusters annotated “COL1A2”, “SCRG1” and “KRT14” as these genes are top discriminating of the three clusters. B: Repartition of the cells incubated (red) or not (grey) with the virus. C: Repartition of naïve, bystander and infected cells in the three clusters.

D: Venn diagram representing genes that are modulated in bystander versus naïve cells and infected versus bystander cells. The pattern of expression of the genes commonly regulated in the two differential analysis is presented as a heat-map. By convention, upregulated genes are identified in red and down regulated genes are identified in green. E: Ingenuity Pathways Analysis showing the upstream regulators describing differentially expressed genes in bystander versus naïve cells and infected versus bystander cells. Orange: pathway activated; blue: pathway inhibited. F: Example of genes part of the IFNγ pathway inversely regulated in bystander versus naïve cells and infected versus bystander cells. Only four out of 44 genes (9.1%) are regulated in the same direction in the two conditions. These genes are noted: *.

FIG. 11: Single-cell transcriptomic analysis of TNBC infected with VV (experiment 2 of example 2). Cells from TNBC were either mock infected or infected with VV (MOI=5). Six hours later the cells were subjected to the 10× Genomics single-cell protocol, followed by sequencing. According to the number of clusters to be identified, colored dots were used. A: UMAP plot representing the four clusters annotated “SPP1”, “CA2”, “COL1A2” and “MDH1” as these genes are top discriminating of the four clusters. B: Repartition of the cells incubated (red) or not (grey) with the virus. C: Repartition of naïve, bystander and infected cells in the four clusters. D: Venn diagram representing genes that are modulated in bystander versus naïve cells and infected versus bystander cells. The pattern of expression of the genes commonly regulated in the two differential analysis is presented as a heat-map. By convention, upregulated genes are identified in red and down regulated genes are identified in green. E: Ingenuity Pathways Analysis showing the upstream regulators describing differentially expressed genes in bystander versus naïve cells and infected versus bystander cells. Orange: pathway activated; blue: pathway inhibited. F: Example of genes part of the IFNγ pathway inversely regulated in bystander versus naïve cells and infected versus bystander cells. Only one out of 17 genes (5.9%) are regulated in the same direction in the two conditions. This gene is noted: *.

FIG. 12: Analysis of the differentially-expressed genes in bystander versus infected cells.

A: Venn diagram of the differentially-expressed genes in bystander versus infected cells in experiments 1 and 2 of example 2. A total of 125 genes are commonly regulated. Ingenuity Pathways Analysis showed that these genes were consistent with an activation of the pathways regulated by TGFβ1, CTNNB1, LPS, TNF and IL1β. The z-score for each pathway is presented.

B: Comparison of the potentially “antiviral genes” in the present study and in the studies of Sivan et al. and Beard et al.

FIG. 13: Analysis of the differentially-expressed genes in bystander versus infected cells using the “conserved markers” strategy.

Venn diagram of the differentially expressed genes in bystander versus infected cells in experiments 1 and 2 of example 2, using a conserved marker analysis. The 7 commonly regulated genes in the two experiments are indicated. ENSCAFG00000032813 and ENSCAF00000031808 are canine genes without human homologs.

### DETAILED DESCRIPTION OF THE INVENTION

Current standard cancer therapies include among others surgery, radiotherapy and chemotherapy. Viral therapy provides an additional tool to treat cancer. Approaches to viral therapy are at least twofold. A first approach includes the use of non-destructive viruses to introduce genes into cells. The rationale of this type of therapy is to selectively provide tumor cells with a biological activity that is lacking or is much lower in the normal cells and which renders the tumor cells sensitive to certain drugs. Another approach to viral therapy to treat cancerous cells involves direct inoculation of tumor with attenuated viruses. Attenuated viruses can exhibit a reduced virulence yet are able to actively multiply and may ultimately cause the destruction of infected cells, in particular of infected cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune system responses.

The present invention more particularly relates to “oncolytic viruses” and to methods of assessing the sensitivity or resistance of a subject having a cancer to an oncolytic virus, methods of selecting a treatment comprising an oncolytic virus efficient against the cancer of a subject and methods of monitoring in a subject the response to a cancer treatment comprising an oncolytic virus, and if required of stopping or adapting the treatment. Oncolytic viruses are herein defined as genetically engineered or naturally occurring viruses, including attenuated version thereof, that selectively replicate in and kill cancer cells without harming the normal tissues.

Inventors worked on freshly-isolated primary cells from low-grade and high-grade canine breast carcinomas and used bulk and single cell RNASeq to analyze events associated with vaccinia virus (VV) infection and to characterize genes potentially interfering with VV cycle. They discovered and herein reveal for the first time that the expression of a specific gene (biomarker of interest) interferes with VV replication and thus affects its therapeutic activity. In a particular aspect, this gene of interest is selected from DDIT4 (DNA Damage Inducible Transcript 4), SERPINE1 (Serpin family E member 1), BHLHE40 (Basic Helix-Loop-Helix Family Member E40), HAS2 (Hyaluronan Synthase 2), MT2A (Metallothionein 2A), AMOTL2 (Angiomotin-like 2), PTRF (Polymerase I and transcript release factor), SLC20A1 (Sodium-dependent phosphate transporter 1), ZYX (Zyxin), CDKN1A (Cyclin-dependent kinase inhibitor 1A), CYP1B1 (Cytochrome P450 Family 1 Subfamily B Member 1), LIF (Leukemia inhibitory factor), NEDD9 (Neural Precursor Cell Expressed, Developmentally Down-Regulated 9), NUAK1 (NUAK family SNF1-like kinase 1 or AMPK-related protein kinase 5), PLAU (Urokinase-type plasminogen activator), THBS1 (Thrombospondin 1), DUSP6 (Dual Specificity protein Phosphatase 1), APEX1 (Apurinic/Apyrimidinic Endodeoxyribonuclease 1) and TBCB (Tubulin Folding Cofactor B). In another particular aspect, the gene is DUSP1 (Dual Specificity Phosphatase 1). In another particular and preferred aspect, the gene is selected from DDIT4, SERPINE1, BHLHE40 and HAS2. In again another particular and preferred aspect, the gene is selected from DDIT4, DUSP6, APEX1 and TBCB. In a further particularly preferred aspect, the gene is DDIT4.

In the below description of the invention, the following terms will be employed and are intended to be defined as indicated below.

As indicated previously, “oncolytic viruses” are herein defined as genetically engineered or naturally occurring viruses, including attenuated version thereof, that selectively replicate in and kill cancer cells without harming the normal tissues in the context of a viral treatment (viral therapy). Viruses for use in the context of the invention, in particular in methods provided herein, include, but are not limited to, a poxvirus, including a vaccinia virus, for example selected from a Lister, a Copenhagen and a Western Reserve (WR) strain, and any attenuated version thereof. Attenuation of a virus means a reduction or elimination of deleterious or toxic effects to a host upon administration of the virus compared to an un-attenuated virus. As used herein, a virus with low toxicity, virulence or pathogenicity means that upon administration a virus does not accumulate in organs and tissues in the host to an extent that results in damage or harm to organs, or that impacts survival of the host to a greater extent than the disease being treated does. The LIVP (Lister virus from the Institute for Research on Virus Preparations, Moscow, Russia) is an example of attenuated Lister strain.

The “cancer” or “tumor” may be any kind of cancer or neoplasia. In a particular aspect, the cancer is a metastatic cancer or a cancer involving an unresectable tumor.

The cancer is typically selected from a carcinoma, a sarcoma, a lymphoma, a melanoma, a paediatric tumour [such as neuroblastomas, ALK (anaplastic lymphoma kinase) lymphoma, osteosarcomas, medulloblastomas, glioblastomas, ependymomas, soft tissue sarcoma, acute myeloid leukemia, and acute lymphoblastic leukemia], and a leukaemia (also herein identified as “leukaemia tumor”).

The cancer is preferably selected from a breast cancer, in particular a breast cancer comprising triple negative carcinoma cells (also herein identified as “triple negative carcinoma cancer” or “TNBC”), a colon cancer, a skin cancer, in particular a melanoma, a lung cancer, a glioblastoma multiform, an osteosarcoma, a soft tissue sarcoma, an ovarian cancer, a prostate cancer, a lymphoma, and an acute myeloid leukemia, preferably a metastatic cancer or a cancer involving an unresectable tumor.

In a particular aspect wherein the gene/protein of interest is DDIT4/DDIT4, the cancer is preferably selected from a breast cancer, in particular a breast cancer comprising triple negative carcinoma cells (also herein identified as “triple negative carcinoma cancer” or “TNBC”), a colon cancer, a skin cancer, in particular a melanoma, a lung cancer, a glioblastoma multiform, an ovarian cancer, and an acute myeloid leukemia, preferably a metastatic cancer or a cancer involving an unresectable tumor.

As used herein, the “subject” or “patient” is an animal, in particular a mammal. The mammal may also be a primate or a domesticated animal such as a dog or a cat. In a particular embodiment, the primate is a human being, whatever its age or sex. The patient typically has a cancer or tumor. Unless otherwise specified in the present disclosure, the tumor is a cancerous or malignant tumor. A particular subpopulation of subjects is composed of subjects suffering from non-metastatic cancer. Another particular subpopulation of subjects is composed of subjects having metastases. In a particular aspect, the subject is a subject who has not been previously exposed to a treatment of cancer or a subject who has received the first administration of anti-cancer agent. In another particular aspect, the subject is a subject who has been previously exposed to a treatment of cancer, for example a subject who has received the administration of at least two or three, therapeutic dose(s) of a treatment of cancer, i.e. of a molecule or agent/product for treating the cancer or tumor, typically of a product comprising or consisting in an oncolytic virus. In a further particular aspect, the subject is a subject who has undergone at least partial resection of the cancerous tumor.

In the context of the present invention, a particular subpopulation of subjects is a subpopulation of subjects suffering from breast cancer, in particular from triple negative carcinoma cancer (TNBC). In a particular aspect of the invention, the subject is suffering of metastatic breast cancer. In the context of breast, in particular of TNBC, and/or ovarian cancer, a particular subpopulation of subjects is composed of subjects having cancer cells that do not express at least one, for example at least two, three or four, gene(s) selected from the gene encoding estrogen receptor (ER), the gene encoding progesterone receptor (PR), the gene encoding HER2/neu, the gene encoding BRCA1, the gene encoding BRCA2, or that do not express the ER, PR, HER2/neu, BRCA1 and BRCA2 genes. Another particular subpopulation of subjects is composed of subjects suffering of a breast and/or ovarian cancer with cancer cells having a mutation within a gene selected from a gene encoding TP53, KRAS, BRAS, and PI3kinase, for example within at least two or three genes, or within each of said four genes. An additional particular subpopulation of subjects is composed of subjects suffering of a breast and/or ovarian cancer who do not respond to hormone therapy.

The invention may be used both for an individual subject and for an entire population of subjects. The subject can be a subject at risk, or suspected to be at risk, of developing a specific cancer, for example a subject with a familial history of cancer, for example of TNBC.

The subject can be asymptomatic, or present early or advanced signs of a cancer. Typically, the subject is asymptomatic or present early signs of a cancer. Typically, the subject exhibits no cancer symptom but is eligible for a clinical study or trial concerning a cancer.

“Treatment” means any manner in which the symptoms of a condition, disorder or disease, in particular cancer, are ameliorated or otherwise beneficially altered. Treatment also encompasses any pharmaceutical use of the viruses described and provided herein. Amelioration or alleviation of symptoms associated with a disease refers to any lessening, whether permanent or temporary, lasting or transient of symptoms that can be attributed to or associated with a disease. Similarly, amelioration or alleviation of symptoms associated with administration of a virus refers to any lessening, whether permanent or temporary, lasting or transient of symptoms that can be attributed to or associated with an administration of the virus for treatment of a disease. Typically, any of the symptoms, such as the tumor, metastasis thereof, the vascularization of the tumors or other parameters by which the disease is characterized are reduced, ameliorated, prevented, placed in a state of remission, or maintained in a state of remission.

As used herein, an effective amount of a virus or compound for treating a particular disease is an amount that is sufficient to ameliorate, or in some manner reduce the symptoms associated with the disease. Such an amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms. An effective amount of a therapeutic agent for control of viral unit numbers or viral titer in a patient is an amount that is sufficient to prevent a virus introduced to a patient for treatment of a disease from overwhelming the patient's immune system such that the patient suffers adverse side effects due to virus toxicity or pathogenicity. Such side effects can include, but are not limited to fever, abdominal pain, aches or pains in muscles, cough, diarrhea, or general feeling of discomfort or illness that are associated with virus toxicity and are related to the subject's immune and inflammatory responses to the virus. Side effects or symptoms can also include escalation of symptoms due to a systemic inflammatory response to the virus, such as, but not limited to, jaundice, blood-clotting disorders and multiple-organ system failure. Such an amount can be administered as a single dosage or can be administered according to a regimen, whereby it is effective. The amount can prevent the appearance of side effects but, typically, is administered in order to ameliorate the symptoms of the side effects associated with the virus and virus toxicity. Repeated administration can be required to achieve the desired amelioration of symptoms.

Implementations of the methods of the invention may involve obtaining a biological sample from a subject, typically a sample from which a sample of a nucleic acid of interest may be obtained and/or the expression of a protein of interest, in particular a protein selected from DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in particular from DDIT4, DUSP6, APEX1 and TBCB, may be analyzed (detected and preferably measured).

The sample may be a solid sample, typically a tumor sample, for example a tumor tissue (biopsy or surgical specimen) or a tumor cell, or a fluid sample.

A fluid sample may be a blood, urine, plasma, serum, lymphatic fluid, spinal fluid, pleural effusion, ascites, sputum, or a combination thereof. The sample is typically a blood sample or a derivative thereof.

A preferred sample is selected from a tumor sample, a blood sample, a serum sample, a plasma sample and a derivative thereof. The tumor tissue is typically a histological section routinely processed for histological evaluation by chemical fixation (for example formalin-fixed/paraffin-embedded) or freezing. In a preferred aspect, the tissue sample submitted for processing and embedding should not excess 3-6 mm, preferably 4 or 5 mm, in thickness. After chemical fixation, the tissue sample is typically dehydrated in alcohol(s) followed by infiltration by for example melted paraffin. The tissue may then by sectioned (typically cut into sections of 4-5 μm thickness) before staining with one or more pigments, and slide mounted. Hematoxylin is used to stain nuclei blue, while eosin stains cytoplasm and the extracellular connective tissue matrix pink. There are hundreds of various other techniques, well known by the skilled person, which have been used to selectively stain cells. Other compounds used to color tissue sections include safranin, Oil Red O, Congo red, silver salts and artificial dyes.

The tumor cell from the biological sample is for example a biopsied cell or a cell from a bodily fluid. In a particular aspect of the invention where the cancer is a breast or ovarian cancer, the tumor cells are preferably epithelial cells from the breast or ovarian tumor.

In a particular and preferred aspect, the herein described methods comprise a step of determining, in a biological sample from a subject, the presence or absence of, and preferably, if present, the basal expression level of, and/or percentage of cells expressing, a protein/mRNA encoded by a gene of interest (as herein described).

In a particular aspect of the invention wherein the protein is to be extracted from a tissue or cell culture, anyone of the three following methods can be advantageously performed:

If the biological sample is a tissue sample, typically a tumor tissue sample, the tissue material is to be grinded for example by Potter's apparatus. The bursting of the cells is completed by osmotic shock or by sonication which will lyse the membranes of the cells. In order not to denature the proteins, the method is preferably performed in buffered medium and at 0° C. (ice). The cell debris from the thereby obtained cellular homogenate are removed by centrifugation. The solubilization of the proteins (when present) is preferably performed in a saline solution, thereby obtaining a crude extract thereof.

If the biological sample is a cell sample, typically a tumor cell sample isolated from a tissue sample, a cell sorter (usually a cytofluorimeter) is preferably used. Antibodies specifically recognize a cell population and will mark it with a fluorochrome to which they are coupled. The apparatus therefore selects fluorescent cells.

If the protein of interest is to be extracted from a particular organelle, a cell fractionation step is typically carried out, i.e. the different constituents of the cell are separated by ultracentrifugation. A protein of interest is typically purified from its particular properties: solubility, ionic charge, size and affinity.

In the context of the present invention, a semi-quantitative immunohistochemical assay is preferably performed to determine protein expression level.

Immunohistochemistry (IHC) technology may be applied as a semi-quantitative tool with a scoring system reflective of intensity of staining, advantageously in conjunction with percentage of stained tumor cells.

IHC measures the level of protein overexpression, while fluorescence in situ hybridization (FISH) may be used to identify specific DNA or RNA molecules and quantify the level of gene amplification. The antibody staining methods often require the use of frozen section histology. Together IHC and FISH are the most commonly used methods of determining a particular protein status in routine diagnostic settings.

Flow cytometry may also be used to detect and measure physical and chemical characteristics of a population of cells, in particular to count cells or to detect a specific protein. A sample containing cells is suspended in a fluid and injected into the flow cytometer instrument. The sample is focused to ideally flow one cell at a time through a laser beam and the light scattered is characteristic to the cells and their components. Cells are often labelled with fluorescent markers so that light is first absorbed and then emitted in a band of wavelengths. Tens of thousands of cells can be quickly examined and the data gathered are processed by a computer. A flow cytometry analyzer is advantageously usable to provide quantifiable data from a sample. Other instruments using flow cytometry include cell sorters which physically separate and thereby purify cells of interest based on their optical properties.

In another particular aspect, the DNA or mRNA is subsequently extracted or purified from the sample prior to genotyping analysis. Any method known in the art may be used for DNA or mRNA extraction or purification. Suitable methods comprise inter alia steps such as centrifugation steps, precipitation steps, chromatography steps, dialyzing steps, heating steps, cooling steps and/or denaturation steps. For some embodiments, a certain DNA or mRNA content in the sample may have to be reached. DNA or mRNA content can be measured for example via UV spectrometry as described in the literature. DNA amplification may be useful prior to the genotyping analysis step. Any method known in the art can be used for DNA amplification. The sample can thus be provided in a concentration and solution appropriate for the genotyping analysis.

Provided are an in vitro or ex vivo (predictive) methods of determining or assessing [including (but not restricted to) predicting] the sensitivity or resistance of a subject having a cancer to an oncolytic virus. This permit, for example, selection of an appropriate therapy, typically viral therapy, or an adaptation/optimization of the therapy.

The term assessing (or determining) is intended to include quantitative and qualitative determination in the sense of obtaining an absolute value for the activity of a product (gene, protein or cell of interest herein considered as the biomarker of interest), and also of obtaining an index, ratio, percentage, visual or other value indicative of the level of the activity. Assessment can be direct or indirect.

By “sensitivity” or “responsiveness” is intended herein the likelihood that a patient positively responds or will positively respond (“sensitive subject” or “responsive subject”/“sensitive tumor” or “responsive tumor”) to a viral therapy/treatment, typically to a viral therapy involving an oncolytic virus such as a vaccinia virus. Typically, a patient or tumor that responds favorably to a treatment with a therapeutic virus means that treatment of a tumor with the virus will cause the tumor to slow or stop tumor growth, or cause the tumor to shrink or regress. This patient or tumor is herein identified as having a responder profile.

By “resistant” is intended herein the likelihood that a patient or its tumor does not respond or will not respond (“resistant subject”/“resistant tumor”) to a viral therapy, typically to a viral therapy involving an oncolytic virus such as a vaccinia virus. A resistant tumor is a tumor for which a therapeutic virus is not effective against in vivo. A resistant patient or tumor is herein identified as having a non-responder profile.

Predictive methods of the invention can be used clinically by the medicinal practitioner to make treatment decisions by choosing as soon as possible the most appropriate treatment modalities/regimens for a particular patient. These predictive methods allow determining the likelihood that a patient will exhibit a (at least partially) positive clinical response or a negative clinical response to treatment with an oncolytic virus and constitute a valuable tool for predicting whether a patient is likely to respond favorably to the viral therapy.

A particular method comprises a step of determining, in a biological sample from a subject, the expression, or on the contrary lack of expression, of at least one gene of interest, typically the presence or absence of at least one protein/mRNA encoded by a gene of interest, and/or the percentage of cells expressing at least one protein encoded by a gene of interest, the gene being selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in particular from DDIT4, DUSP6, APEX1 and TBCB, possibly a set or panel of proteins/mRNA respectively encoded by a gene selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, thereby assessing whether the subject having a cancer is sensitive or resistant to the oncolytic virus, the absence of the DDIT4 protein/mRNA or a level thereof identical to or below a reference expression level, and/or the absence of cells expressing the DDIT4 protein or a percentage thereof identical to or below a reference percentage of cells expressing the DDIT4 protein, being for example associated to sensitivity of the subject having any one of the herein described cancer to oncolytic virus, whereas the presence of the DDIT4 protein/mRNA or a level thereof above a reference expression level, and/or of the presence of cells expressing the DDIT4 protein or a percentage thereof above a reference percentage of cells expressing the DDIT4 protein, being associated to resistance of the subject having any one of the herein described cancer to oncolytic virus.

Another particular method comprises a step a) of determining, in a biological sample from a subject, the presence or absence of, and if present, the expression level of, and/or percentage of cells expressing, at least one protein/mRNA encoded by a gene of interest selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in particular from DDIT4, DUSP6, APEX1 and TBCB, and, when the expression level of, and/or percentage of cells expressing, the at least one protein/mRNA is determined, a step b) of comparing said expression level to a reference expression level, and/or said percentage of cells to a reference percentage of cells, thereby assessing whether the subject having a cancer is sensitive or resistant to the oncolytic virus.

In a particular aspect, the protein/mRNA expression level determined in step a) is the protein/mRNA basal expression level in the subject, and the percentage of cells expressing the protein determined in step a) is the basal percentage of cells expressing the protein in the subject, and step b) comprises comparing said protein/mRNA basal expression level to a protein/mRNA response expression level in the subject as determined after an administration to said subject of the oncolytic virus, the mRNA response expression level being possibly used as the reference expression level, and/or comparing said basal percentage of cells to a percentage of cells expressing the protein in the subject as determined after an administration to said subject of the oncolytic virus, said percentage of cells being possibly used as the reference percentage of cells. In another particular aspect, the protein/mRNA basal expression level, or basal percentage of cells expressing the protein, is determined before any step of cancer treatment applied to the subject or at about the same time as, typically when, beginning a cancer treatment.

Any method known in the art can be used for assessing the expression of a gene in a tumor. Examples of techniques which can be used to detect and measure RNA levels include microarray analysis, quantitative PCR, Northern hybridization, or any other technique for the quantitation of specific nucleic acids.

Examples of methods for detecting and measuring protein expression levels which can be used include, but are not limited to, IHC and flow cytometry, as explained herein above, but also microarray analysis, ELISA assays, Western blotting, or any other technique for the quantitation of specific proteins.

Microarray analysis may involve an array, i.e. a collection of elements such as proteins, nucleic acids or cells, suspended in solution or spread out on a surface, for example affixed to support such as a chip, a tube, a slide, a flask, a microbead or any other suitable laboratory apparatus.

As used herein, a “reference expression level or value” or “control expression level or value” can be an absolute value; a relative value; a value that has an upper and/or lower limit; a range of values; an average value; a median value; a mean value; a statistic value; a cut-off or discriminating value; or a value as compared to a particular control or baseline value.

A reference value can be based on an individual sample value, such as for example, a value obtained from a sample from the individual tested but at an earlier point in time, or a value obtained from a sample from a subject other than the subject tested or a “normal” subject that is a subject identified has having a healthy status or a subject not diagnosed with any cancer.

The reference value identifies the sub-population with a predetermined specificity and/or a predetermined sensitivity based on an analysis of the relation between the parameter values and the known clinical data of the reference population (which can be for example a healthy control population, or any other control population diagnosed with an identified disease distinct of cancer, or distinct of the specific cancer under consideration) and of the population of the subjects of interest. The discriminating values determined in this manner are valid for the same experimental setup in future individual tests.

For example, the reference value can be expressed as a concentration of the biomarker in the biological sample of the tested subject for a particular specificity and/or sensitivity, or can be a normalized cut-off value expressed as a ratio for a particular specificity and/or sensitivity.

As well known by the skilled person, the reference expression value/level will vary depending in particular on the nature of the studied biomarker, on the nature of tools used to measure the biomarker (typically protein/mRNA/cell) expression, and on the nature of the evaluated biological sample.

If a higher or lower sensitivity and/or specificity is/are desired, the cut-off value can easily be changed by the skilled person, for example using a different reagent for a particular gene, protein or cell of interest.

In a particular aspect, the biomarker of interest is a protein, and a concentration of this protein per mg of tumor, or a protein expression score/grade, is characteristic of tumors which do not respond favorably to viral therapy, whereas a higher or lower (depending on the nature of the biomarker) concentration or score/grade, is characteristic of tumors which responds favorably to viral therapy, and viral therapy can be initiated.

In another particular aspect of the invention, a DDIT4 concentration/level above a DDIT4 reference concentration/level is associated to a non-responder profile, whereas a DDIT4 concentration/level at, or below, the DDIT4 reference concentration/level is associated to a responder profile allowing initiation or continuation of viral therapy.

Thanks to the present invention, a proportion/percentage (%) of tumor cells (such as any type of cancer cells as herein described) expressing a biomarker such as DDIT4 is associated to a non-responder profile, whereas a lower proportion/percentage of such tumor cells expressing DDIT4 is associated to a responder profile.

In a particular aspect of the invention, both the biomarker (protein/mRNA) expression and the proportion/percentage (%) of tumor cells expressing the biomarker are determined/evaluated. A quantity of biomarker “above the control value” or “higher than the control value”, or on the contrary “below the control value”, may mean a significant statistical increase/decrease, for example of at least 2 standard deviations.

In a particular method of assessing the sensitivity or resistance of a subject having a cancer to an oncolytic virus herein described, the cancer is a breast or ovarian cancer and a DDIT4 protein/mRNA basal expression level above a DDIT4 protein/mRNA reference expression level, or a percentage of cells expressing a DDIT4 protein above a reference percentage of cells, is indicative of a resistance of the subject to the oncolytic virus, whereas a DDIT4 protein/mRNA basal expression level identical to or below said DDIT4 protein/mRNA reference expression level, or a percentage of cells expressing a DDIT4 protein identical to or below said reference percentage of cells, is indicative of a sensitivity of the subject to the oncolytic virus.

Also provided is a method, in particular an in vitro or ex vivo method, of monitoring in a subject the response to a cancer treatment comprising an oncolytic virus (also herein identified as a viral therapy), and if required of stopping or adapting the treatment. This method comprises:
a step a) of determining at a first time point, T0, the expression level of (herein identified as “protein/mRNA reference expression level”), and/or the percentage of cells expressing (herein identified as “reference percentage of cells”), a protein/mRNA encoded by a gene selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLA U, THBS1, DUSP6, APEX1 and TBCB, in particular from DDIT4, DUSP6, APEX1 and TBCB, in a biological sample of a subject having a cancer, before any step of cancer treatment applied to the subject or at about the same time as, typically when, beginning a cancer treatment in the subject, typically a treatment comprising an oncolytic virus, or after a step of cancer treatment applied to the subject, typically a treatment comprising an oncolytic virus, a step a′) of determining in a biological sample of the subject having a cancer obtained at a different time point, for example T1, T1 following T0, the protein/mRNA response expression level and/or percentage of responding cells expressing the protein/mRNA, after the administration to said subject of a first, or additional, therapeutic dose of an oncolytic virus for treating the cancer, and
a step b) of comparing said protein/mRNA response expression level to said protein/mRNA reference expression level and/or to a protein/mRNA reference expression level in a control population, and/or of comparing said percentage of responding cells expressing the protein to said reference percentage of cells and/or to a reference percentage of cells in a control population, a protein/mRNA response expression level identical to or below the protein/mRNA reference expression level(s), and/or a percentage of responding cells expressing the protein identical to or below the reference percentage of cells, being the indication that the oncolytic virus will be efficient as such against the cancer of the subject, whereas a protein/mRNA response expression level above the protein/mRNA reference expression level(s), and/or a percentage of responding cells expressing the protein above the reference percentage of cells, being the indication that an oncolytic virus will not be efficient alone in the subject, and, if the oncolytic virus is not efficient as such,
a step c) of stopping or adapting the treatment of the subject's cancer, for example by selecting a treatment combining said oncolytic virus with an additional compound, in particular an additional protein selected typically from DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in particular from DDIT4, DUSP6, APEX1 and TBCB, or a treatment comprising a therapeutic recombinant oncolytic virus.

In a particular aspect, steps a) and a′) are reproduced at a plurality of time points to monitor the progress of a cancer treatment during a period of time, and the method includes one or several steps of comparing a protein/mRNA expression level to a previously measured protein/mRNA expression level and/or one or several steps of comparing a percentage of cells to a previously measured percentage of cells.

The time between the first time point and the different (at least second) time point can be about 30 minutes, about 1 hour, about 6 hours about 12 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, and about 1 month.

In certain aspect, the biological samples can be obtained from the same anatomical site.

In some examples, the step of determining whether the level of expression of the at least one selected marker (protein, mRNA or cell) in a biological sample from a subject has decreased, increased, or remained substantially the same, as compared to the expression of the same at least one selected marker in a biological sample obtained at a later time point from the subject, can be performed by comparing quantitative or semi-quantitative results obtained from the measuring steps a) and a′). In some examples, the difference in expression of the same selected marker between the biological samples can be about less than 2-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold or greater than about 100-fold.

Further herein provided is a method, in particular an in vitro or ex vivo method, of selecting an appropriate or optimal treatment of cancer for a subject, typically a treatment comprising an oncolytic virus efficient against the cancer of a subject. This method comprises, typically in the following order:
a step a) of determining the presence or absence of, and preferably, if present, the basal expression level of, and/or basal percentage of cells expressing, at least one protein/mRNA encoded by a gene selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in particular from DDIT4, DUSP6, APEX1 and TBCB, possibly several proteins/mRNAs, in a biological sample of a subject having a cancer, before any step of cancer treatment, in particular of cancer treatment comprising an oncolytic virus, applied to the subject or at about the same time as, typically when, beginning such a cancer treatment in the subject,
a step a′) of determining, in a biological sample of the subject having a cancer, the protein(s)/mRNA(s) response expression level, and/or percentage of cells/mRNAs (respectively) expressing the (each kind of) protein(s)/mRNA(s), after the administration to said subject of at least one therapeutic dose of an oncolytic virus for treating the cancer,
a step b) of:
   comparing said protein/mRNA response expression level to said protein/mRNA basal expression level and/or to a protein/mRNA reference expression level in a control population, and/or of
   comparing said percentage of cells expressing the protein to said basal percentage of cells and/or to a reference percentage of cells in a control population, and
a step c) of selecting an appropriate treatment of the subject's cancer, wherein:
   a protein/mRNA response expression level identical to or below the protein/mRNA basal and/or reference expression level, and/or a percentage of cells expressing the protein identical to or below the basal and/or reference percentage of cells, is the indication that the oncolytic virus will be efficient as such and is to be selected for treating the cancer of the subject, whereas
   a protein/mRNA response expression level above the protein/mRNA basal and/or reference expression level, and/or a percentage of cells expressing the protein above the basal and/or reference percentage of cells, is the indication that the oncolytic virus will not be efficient as such, and that an appropriate treatment of the subject's cancer is to be selected, the appropriate treatment being (consisting in) for example a treatment combining said oncolytic virus with an additional compound, in particular an additional protein selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in particular from DDIT4, DUSP6, APEX1 and TBCB, or a treatment comprising a therapeutic recombinant virus, typically a therapeutic recombinant oncolytic virus as herein described.

The therapeutic (recombinant) virus(es) may be used in combination with an additional (therapeutic) compound, typically an additional therapeutic non-viral compound as herein below described.

Inventors also herein advantageously describe viruses designed for viral therapy (i.e. therapeutic viruses), in particular a recombinant virus. The viral genome of such a virus is modified to carry the genetic information for expression of an agent typically for modulating (in particular inhibiting or, on the contrary, enhancing) the expression of a target gene in target cells, typically in tumor cells.

These viruses have desirables features (resulting in a change of viral characteristics) such as attenuated pathogenicity, reduced toxicity, preferential accumulation in certain cells and tissues, typically tumor tissues, ability to activate an immune response against tumor cells, immunogenicity, ability to lyse or cause tumor cell death, replication competence, expression of exogenous nucleic acids or proteins, and any combination of the foregoing features.

Inventors herein describe an advantageous therapeutic recombinant virus, in particular a therapeutic recombinant oncolytic virus, preferably a recombinant oncolytic vaccinia virus.

In a preferred aspect, this therapeutic recombinant virus designed for gene therapy encodes an agent, for example a nucleic acid or a protein, which inhibits or reduces the level of expression of a marker, typically a gene or a protein, whose level of expression is increased in cells which do not respond favourably to viral therapy such as DDIT4/DDIT4.

In another aspect, the therapeutic recombinant virus designed for gene therapy encodes an agent, for example a nucleic acid or a protein, which enhances the level of expression of a marker, typically a gene or a protein, whose level of expression is decreased in cells which do not respond favourably to viral therapy.

Methods to decrease the level of expression of a protein can include providing a therapeutic virus to a subject or to a cell, where the virus can express a protein that inhibits the expression of the marker.

Methods to increase the level of expression of a protein can include providing a therapeutic virus to a subject or to a cell, where the virus can express a protein that enhances the expression of the marker.

In some aspects, the level of expression of a marker protein or mRNA in a cell can be decreased by providing a therapeutic virus encoding a nucleic acid that reduces the level of expression of a marker whose level of expression is decreased in cells that respond favourably to viral therapy. In such methods the therapeutic agent can include an antisense nucleic acid (DNA or RNA) targeted against a nucleic acid encoding the marker, a small inhibitory RNA (siRNA) targeted against a nucleic acid encoding the marker, a small hairpin RNA (sh-RNA) targeted against a nucleic acid encoding the marker, or a ribozyme targeted against a nucleic acid encoding the marker. Methods to decrease the level of expression of a marker protein using antisense nucleic acids are well known in the art. Antisense sequences can be designed to bind to the promoter and other control regions, exons, introns or even exon-intron boundaries of a gene. Antisense RNA constructs, or DNA encoding such antisense RNAs, can be employed to inhibit gene transcription or translation or both within a host cell, either in vitro or in vivo, such as within a host subject, typically a mammal, including a human subject. Methods to decrease the level of expression of a marker protein using siRNA are well known in the art. For example, the design of a siRNA can be readily determined according to the mRNA sequence encoding of a particular protein.

Some methods of siRNA design and downregulation are further detailed in U.S. Patent Application Publication No. 20030198627.

Methods to decrease the level of expression of a particular protein using a ribozyme are well known in the art. Several forms of naturally-occurring and synthetic ribozymes are known, including Group I and Group II introns, RNaseP, hairpin ribozymes and hammerhead ribozymes (Lewin A S and Hauswirth W W, Trends in Molecular Medicine 7: 221-228, 2001). In some examples, ribozymes can be designed as described in WO 93/23569 and WO 94/02595. U.S. Pat. No. 7,342,111 also describes general methods for constructing vectors encoding ribozymes.

In a particular aspect, the agent is thus a nucleic acid, preferably a nucleic acid selected from a si-RNA, a sh-RNA, an antisense-DNA, an antisense-RNA and a ribozyme.

The nucleic acid typically comprises or consists in a sequence of about 10 nucleotides to about 250 nucleotides, preferably of about 18 nucleotides to about 200 nucleotides, for example of about 10, 15, 20, 25, 30, 35, 40, 45, 50, 60, 65, 70, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245 or 250 nucleotides.

In certain aspects, the heterologous nucleic acid is operatively linked to regulatory elements. Such regulatory elements can include (constitutive or inducible) promoters, enhancers, or terminator sequences. In some examples, the virus contains a regulatory sequence operatively linked to a nucleic acid sequence encoding an agent, such as an agent as described herein above, which reduces the level of expression of a marker whose level of expression is increased in cells which do not respond favourably to viral therapy, or on the contrary which increases the level of expression of a marker whose level of expression is decreased in cells which do not respond favourably to viral therapy, in particular in cells which permit poor viral replication.

A regulatory sequence can for example include a natural or synthetic vaccinia virus promoter. In another aspect, the regulatory sequence can contain a poxvirus promoter. In some examples, strong late promoters can be used to achieve high levels of expression of the foreign genes. Early and intermediate-stage promoters, however, can also be used. In one embodiment, the promoters contain early and late promoter elements, for example the early-late vaccinia p7.5 promoter. In a particular aspect, the therapeutic recombinant virus of the invention is replication competent, i.e. it has an increased capacity to accumulate in targeted tumor tissues, metastases or cancer cells. In exemplary examples, viruses designed for viral therapy can accumulate in a targeted organ, tissue or cell at least about 2-fold greater, at least about 5-fold greater, at least about 10-fold greater, at least about 100-fold greater, at least about 1,000-fold greater, at least about 10,000-fold greater, at least about 100,000-fold greater, or at least about 1,000,000-fold greater, than the accumulation in a non-targeted organ, tissue or cell.

A preferred recombinant virus comprises a nucleic acid for modulating a gene or its expression product in a cell, the gene being selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU and THBS1, DUSP6, APEX1 and TBCB, in particular for inhibiting DDIT4, DUSP6, APEX1 or TBCB.

Methods for the generation of recombinant viruses are well known in the art (e.g., see He et al. (1998) PNAS 95(5): 2509-2514; Racaniello et al, (1981) Science 214: 916-919; Hruby et al, (1990) Clin Micro Rev. 3:153-170; Moss (1993) Curr. Opin. Genet. Dev. 3:86-90; Broder and Earl (1999) MoI. Biotechnol. 13, 223-245; Timiryasova et al. (2001) Biotechniques 31: 534-540). In some examples, genetic variants can be obtained by general methods such as mutagenesis and passage in cell or tissue culture and selection of desired properties, by methods in which nucleic acid residues of the virus are added, removed or modified relative to the wild type. Any of a variety of known mutagenic methods can be used, including recombination-based methods, restriction endonuclease-based methods, and PCR-based methods. Mutagenic methods can be directed against particular nucleotide sequences such as genes, or can be random, where selection methods based on desired characteristics can be used to select mutated viruses. Any of a variety of viral modifications can be made, according to the selected virus and the particular known modifications of the selected virus.

In certain examples, any of a variety of insertions, mutations or deletions of the vaccinia viral genome can be used herein. Such modifications can include insertions, mutations or deletions of one or more genes selected typically from DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1 DUSP6, APEX1 and TBCB, in particular from DDIT4, DUSP6, APEX1 and TBCB.

An example of a virus herein described has one or more expression cassettes removed from the wild-type strain and replaced with a heterologous DNA sequence.

The viruses of the invention can be formulated (in particular can be used to prepare a pharmaceutical composition, typically a medicament) and administered to a subject for treating a cancer or tumor.

Also herein described is a host cell, in particular a mammalian host cell, for example a human host cell, containing a recombinant oncolytic virus according to the invention. The host cell can be a tumor cell and can be derived from a primary tumor or from a metastatic tumor, the tumor being preferably a tumor obtained from the subject to be treated with the recombinant oncolytic virus according to the invention.

When the tumor is a solid tumor, isolation of tumor cells is typically achieved by surgical biopsy. When the cancer is a hematopoietic neoplasm, tumor cells can be harvested by methods including, but not limited to, bone marrow biopsy, needle biopsy, such as of the spleen or lymph nodes, and blood sampling. Biopsy techniques that can be used to harvest tumor cells from a patient include, but are not limited to, needle biopsy, aspiration biopsy, endoscopic biopsy, incisional biopsy, excisional biopsy, punch biopsy, shave biopsy, skin biopsy, bone marrow biopsy, and the Loop Electrosurgical Excision Procedure (LEEP).

Herein described is also a pharmaceutical composition comprising a therapeutic virus according to the invention, in particular a therapeutic recombinant virus, and pharmaceutically acceptable carrier(s) and/or excipient(s), typically a medicament.

Examples of suitable pharmaceutical carriers or excipients are known in the art and include phosphate buffered saline solutions, water, emulsions, such as oil/water emulsions, various types of wetting agents, sterile solutions. Such carriers can be formulated by conventional methods and can be administered to the subject at a suitable dose. Colloidal dispersion systems that can be used for delivery of viruses include macromolecule complexes, nanocapsules, microspheres, beads and lipid-based systems including oil-in-water emulsions (mixed), micelles, liposomes and lipoplexes. An exemplary colloidal system is a liposome. Organ-specific or cell-specific liposomes can be used in order to achieve delivery only to the desired tissue. The targeting of liposomes can be carried out by the person skilled in the art by applying commonly known methods. This targeting includes passive targeting (utilizing the natural tendency of the liposomes to distribute to cells of the RES in organs which contain sinusoidal capillaries) or active targeting (for example by coupling the liposome to a specific ligand, for example, an antibody, a receptor, sugar, glycolipid, protein etc., by well-known methods). In the present methods, monoclonal antibodies can be used to target liposomes to specific tissues, for example, tumor tissue, via specific cell-surface ligands.

The pharmaceutical composition can contain an additional (therapeutic) agent.

The additional therapeutic agent can be an agent that decreases the level of expression of a protein whose level of expression is decreased in cells that respond favourably to viral therapy or an agent that increases the level of expression of a protein whose level of expression is increased in cells that respond favourably to viral therapy.

The additional (therapeutic) agent can be a protein or a nucleic acid and be either natural or artificial. This additional agent is typically a non-viral agent. For example, the agent can be a chemical compound such as an anticancer agent (for example a chemotherapeutic agent such as cisplatin or a monoclonal antibody such as bevacizumab) or an agent used in the context of immunotherapy (for example a PD 1 inhibitor or a PD-L1 inhibitor).

When the cancer is a breast cancer, the additional (therapeutic) agent is typically selected from a therapeutic product classically used in hormonotherapy (such as tamoxifen, fulvestrant, an aromatase inhibitor, etc.), in chemotherapy (such as an anthracycline, a carboplatin, a taxane, 5-FU, a cyclophosphamide, etc.), in immunotherapy (such a PD-L1 inhibitor) or in targeted therapy (such as trastuzumab, lapatinib, etc.).

When the cancer is a ovarian cancer, the additional (therapeutic) agent is typically selected from a therapeutic product classically used in hormonotherapy (such as tamoxifen, an aromatase inhibitor, etc.), in chemotherapy (such as paclitaxel, ifosfamide, cisplatin, vinblastine, etoposide, vincristine, dactinomycin and a cyclophosphamide, etc.), or in targeted therapy (such as bevacizumab, a PARP inhibitor, etc.).

The composition can be a solution, a suspension, an emulsion, a liquid, a powder, a paste, an aqueous composition, a non-aqueous composition, or any combination of such formulations.

Inventors also herein describe a therapeutic virus, in particular a therapeutic recombinant virus, and a pharmaceutical composition comprising such a therapeutic virus, as herein described, for use in medicine, typically for use as a medicament, in particular for use in the prevention or treatment of a cancer, typically a cancer as herein described, in particular a breast cancer, preferably a TNBC, or an ovarian cancer.

The therapeutic virus or pharmaceutical composition may be used in combination with other therapies, preferably another cancer therapy, such as a chemotherapy and/or a radiotherapy.

Also herein described is a method for treating a subject suffering of a cancer as herein described. Such a method typically includes a step of administering the subject with at least one particular agent, in particular a therapeutic recombinant virus or pharmaceutical composition as herein described, in a therapeutically effective amount, possibly any combination thereof.

A therapeutically effective amount of a therapeutic virus of the present invention is the amount which results in the desired therapeutic result, in particular cancer or tumor treatment (as herein defined). For example, a therapeutically effective amount of therapeutic virus can be in the range of about 10⁶ pfu to about 10¹⁰ pfu, preferably of about 10⁷, 10⁸ or 10⁹ pfu to about 10¹⁰ pfu. The skilled person is able to determine suitable therapeutically effective amounts depending on the subject, on the nature of the cancer and on the route of administration.

Therapeutic agents (oncolytic viruses or a oncolytic virus together with an additional therapeutic non-viral agent) can be co-administered to a subject at the same time or at a different time, possibly in multiple cycles over a period of time, such as for several days up to several weeks.

Routes of administering viral therapy (the virus itself or a composition comprising the virus) can include systemic delivery, preferably intravenous delivery, intratumoral administration, enteral or parenteral administration.

A preferred therapeutically effective amount of therapeutic virus can be in the range of about 10⁶ pfu to about 10⁸ pfu when the selected route is the intratumoral route, and a preferred therapeutically effective amount of therapeutic virus can be in the range of about 10⁹ pfu to about 10¹⁰ pfu when the selected route is the intravenous route.

The route can be the intravenous, intradermal, subcutaneous, intramuscular, oral (e.g., inhalation), transdermal (topical), transmucosal, intraperitoneal, intrathecal, intracerebral, intravitreal, epidural, intraarticular, intracavernous, or rectal route.

Inventors also herein describe a kit reagents, devices or instructions for use thereof as well as the use of such a kit, typically for assessing the sensitivity or resistance of a subject having a cancer to an oncolytic virus, or for monitoring in a subject the response to a cancer treatment comprising an oncolytic virus, and, optionally, for preventing or treating the cancer of the subject.

The kit typically comprises at least one, typically at least two, mean(s)/reagent(s) to detect and optionally measure the expression level of at least one marker associated with a favourable or a poor response, in particular a poor response, to viral therapy; and optionally at least one of a reagent or device to obtain a biological sample; a therapeutic agent as herein described, such as at least one therapeutic (recombinant) virus, possibly a plurality of distinct therapeutic viruses; a pharmaceutical composition; a reagent or device to administer viral therapy; a host cell containing a therapeutic virus; reagents to measure the presence of a therapeutic virus in a subject; control tissue (cell line) slide(s); a leaflet providing the marker(s) (typically protein(s)) reference expression level(s), and/or reference percentages of cells expressing a protein selected from DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, preferably from DDIT4, DUSP6, APEX1 and TBCB, in control population(s), or instructions for example for assessing the sensitivity or resistance of a subject having a cancer to an oncolytic virus, for selecting proper treatment comprising an oncolytic virus, for monitoring the response to a cancer treatment comprising an oncolytic virus over duration of the treatment time and/or for administering a therapeutic virus.

Exemplary devices include a hypodermic needle, an intravenous needle, a catheter, a needle-less injection device, an inhaler, and a liquid dispenser such as an eyedropper.

The kit can in particular contain means/reagents to detect and/or measure the expression level of one or more markers associated with a favourable or poor response to viral therapy. Such a kit can comprise means for, or components for, detecting and/or measuring particular protein levels in a biological sample, such as antibodies, in particular monoclonal antibodies, specific to a particular protein; or a means or component for measuring particular mRNA levels in a biological sample, such as nucleic acid probes specific for RNA encoding the marker.

A particular kit comprises at least one antibody used as a detection means, this antibody being specific to a protein; a molecule allowing the antibody detection; and optionally a therapeutic recombinant virus.

The detection means of the kit is preferably selected from the group consisting of at least one antibody specific to DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 or TBCB, preferably specific to DDIT4, DUSP6, APEX1 or TBCB, a molecule allowing the antibody detection; a therapeutic recombinant virus; and, optionally, a leaflet provides the DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and/or TBCB respective reference expression level(s), and/or reference percentages of cells expressing a protein selected from DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in control population(s).

Is in particular described the use of a kit as herein described for assessing the sensitivity or resistance of a subject having a cancer to an oncolytic virus, or for monitoring in a subject the response to a cancer treatment comprising an oncolytic virus, and, optionally, for preventing or treating the cancer of the subject.

Further aspects and advantages of the present invention will be described in the following examples, which should be regarded as illustrative and not limiting.

### EXAMPLES

### Example 1: Single Cell Transcriptomic Analysis to Identify Genes Interfering with Viral Replication

Materials and Methods

Cells

Protocol Describing the Isolation and Culture of the Cells:

Very low passage canine primary cell cultures were provided by Lucioles Consulting. They were derived from a panel of canine primary tissues including normal mammary tissues, hyperplastic lesions, benign tumors, carcinomas in situ and all grades of carcinomas. The tissues were phenotyped using standard histopathology and immunohistochemistry techniques. Cell survival assays were performed as previously described (Martinico et al., 2006). BHK21, MCF7, MDAMB231, HeLa and DDIT4^{+/+} and ^{-/-} MEF cells were obtained and cultured as previously described [Ben Sahra I et al, 2011; Montiel-Equihua C A et al, 2008, Vassaux G et al, 1999; Riesco-Eizaguirre G et al, 2011; Ellisen L W et al, 2002; Hebben M et al, 2007]. Fluorescence imaging and Western blots were performed as previously described [Savary G et al, 2019; Vassaux G et al, 2018], respectively. Quantification of fluorescent cells were performed using the CellQuant program (available at: http://biophytiro.unice.fr/cellQuant/index_html)

Virus

A VACV-Lister strain deleted in the thymidine kinase gene and VACV-Copenhagen recombinants encoding GFP downstream of a synthetic early promoter (VACV-Cop21 and VACV-Cop 32) were described previously (Dimier et al., 2011; Drillien et al., 2004). Virus titration was performed on BHK21 cell monolayers infected for two days and stained with neutral red.

Results

TNBC Canine Cells Show Reduced Sensitivity to VV Compared to Non-Triple-Negative Carcinoma Cells.

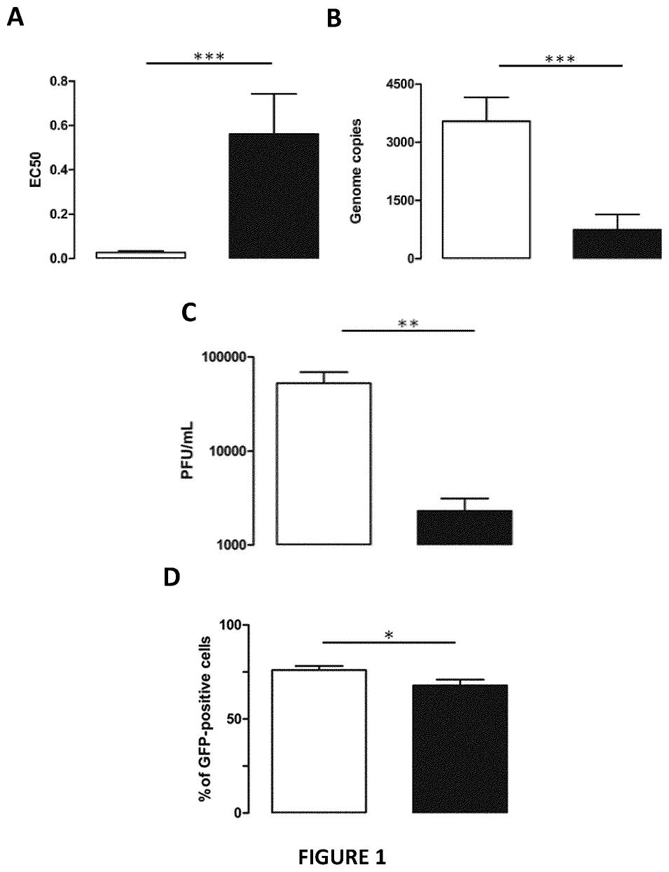

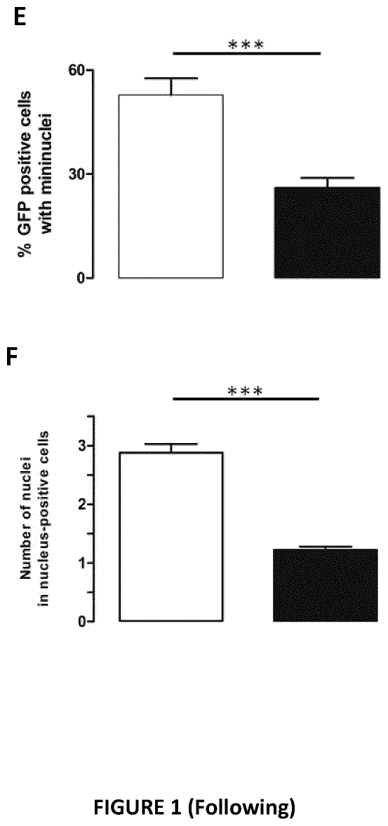

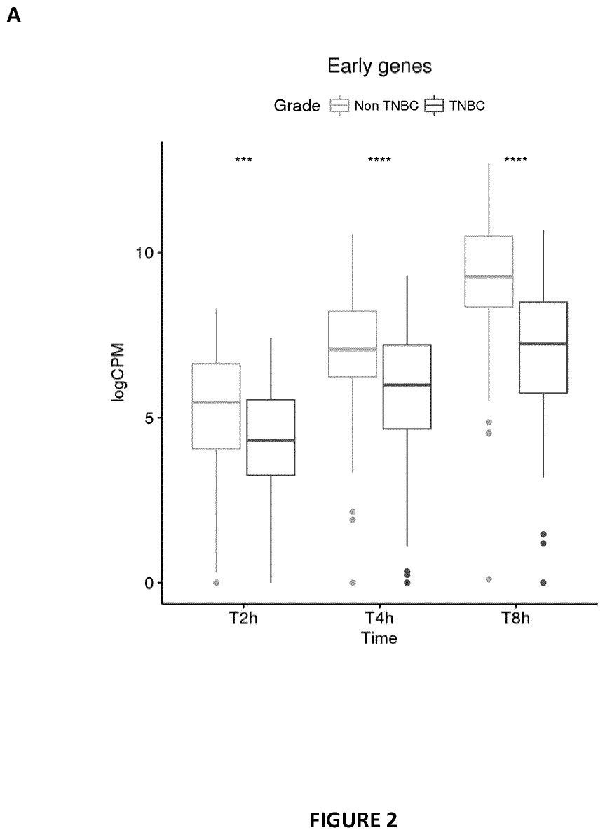

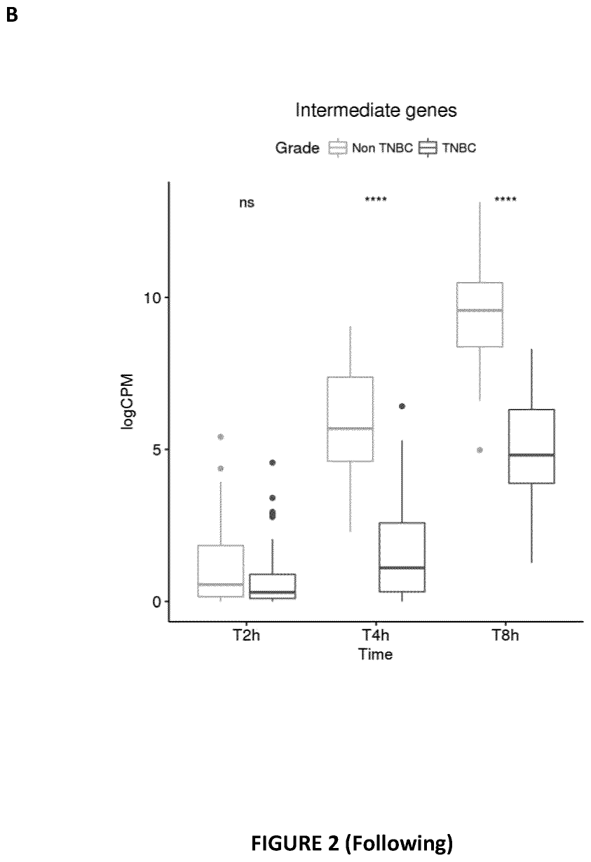

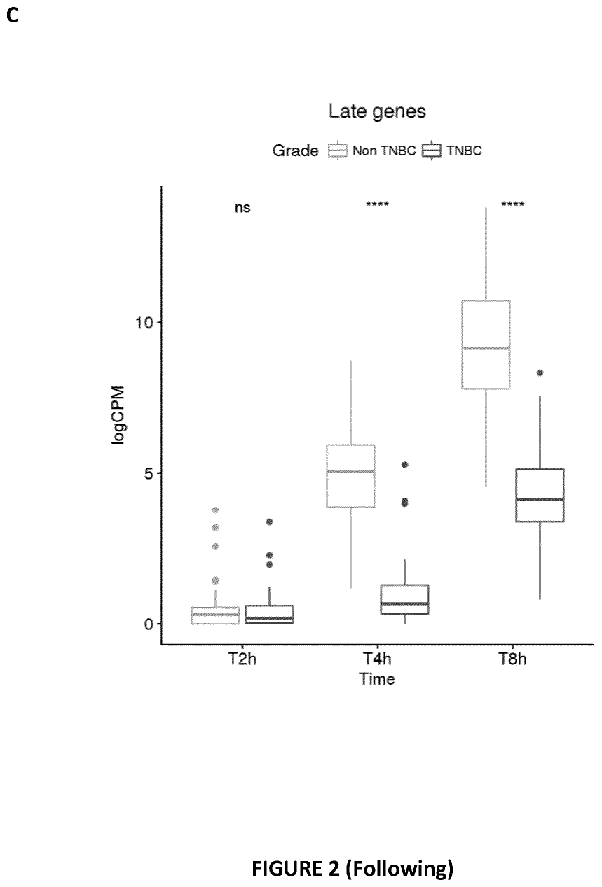

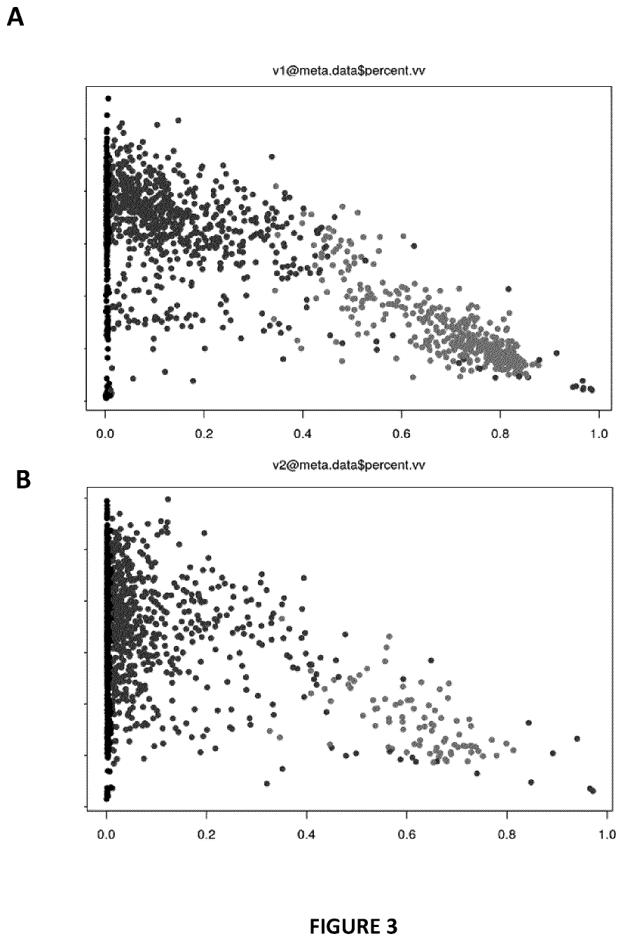

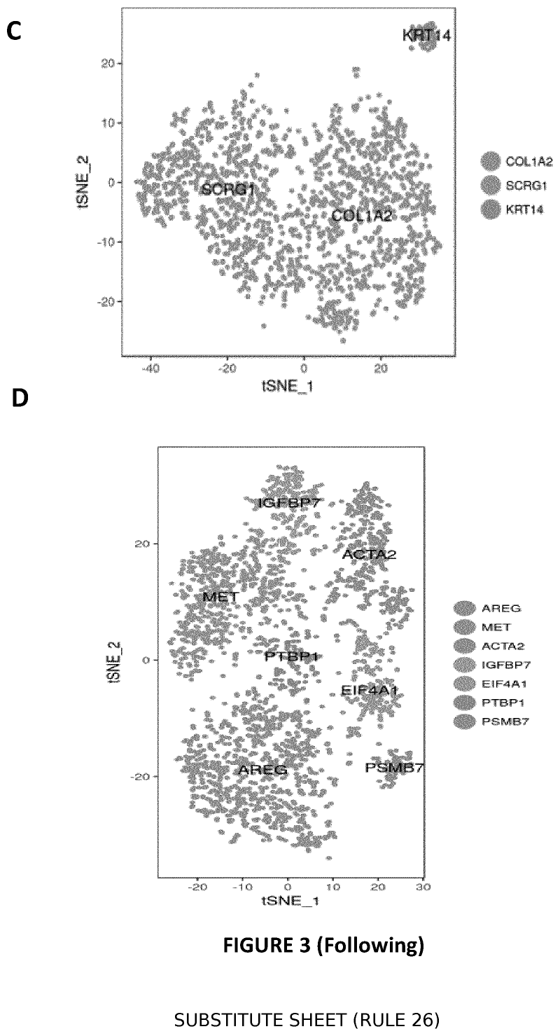

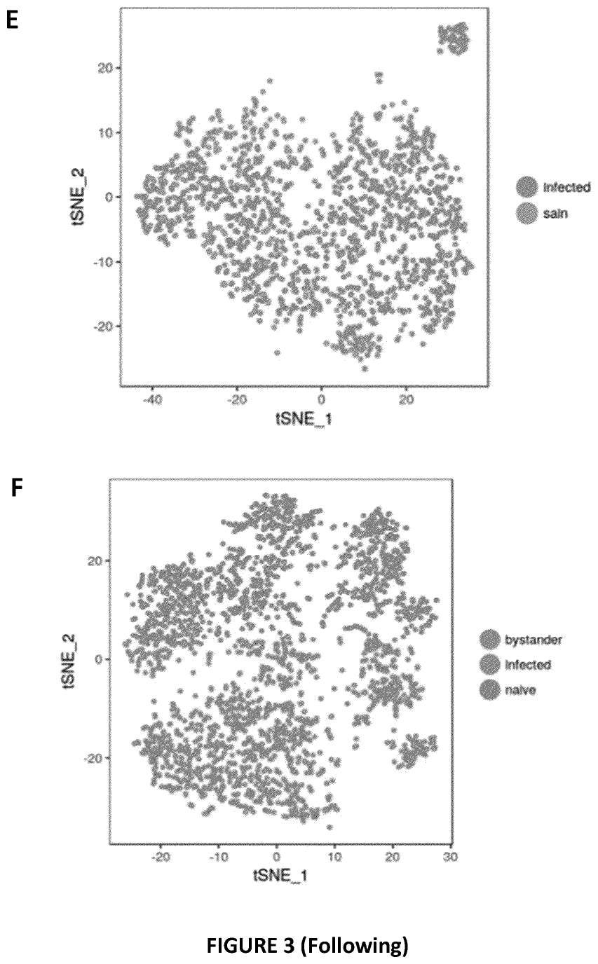

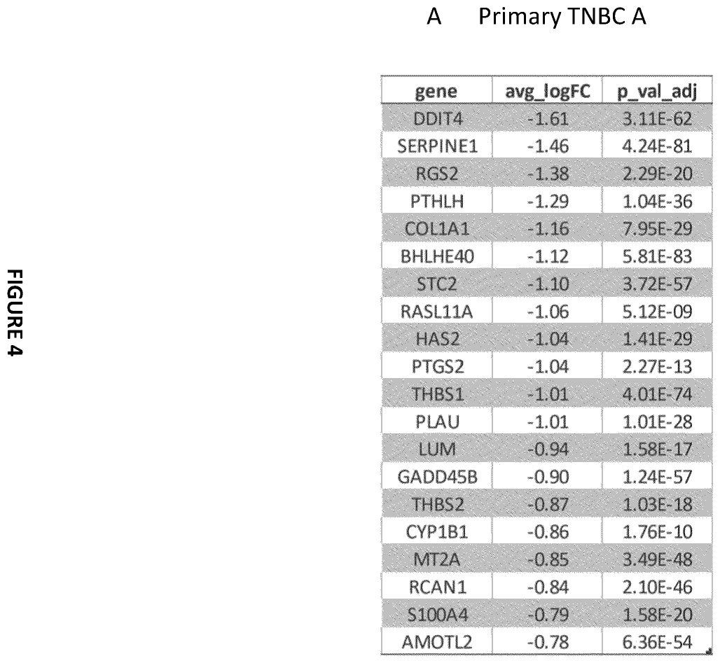

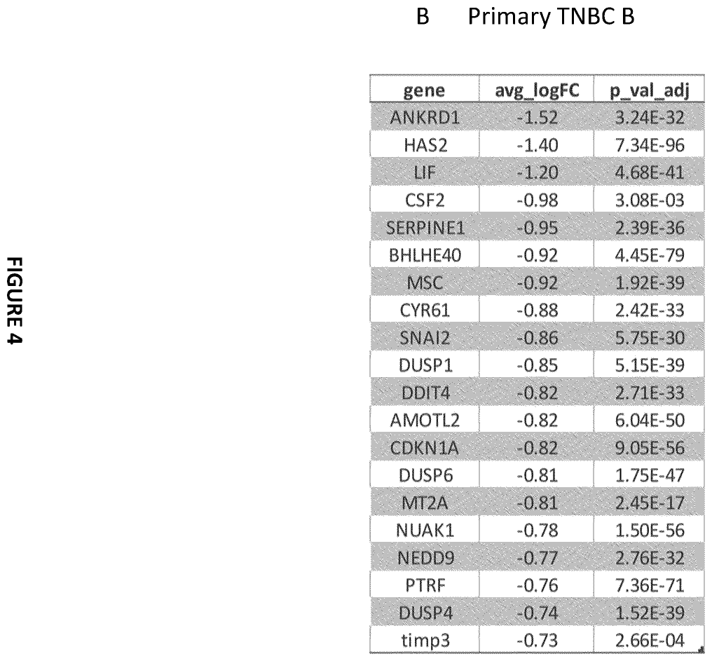

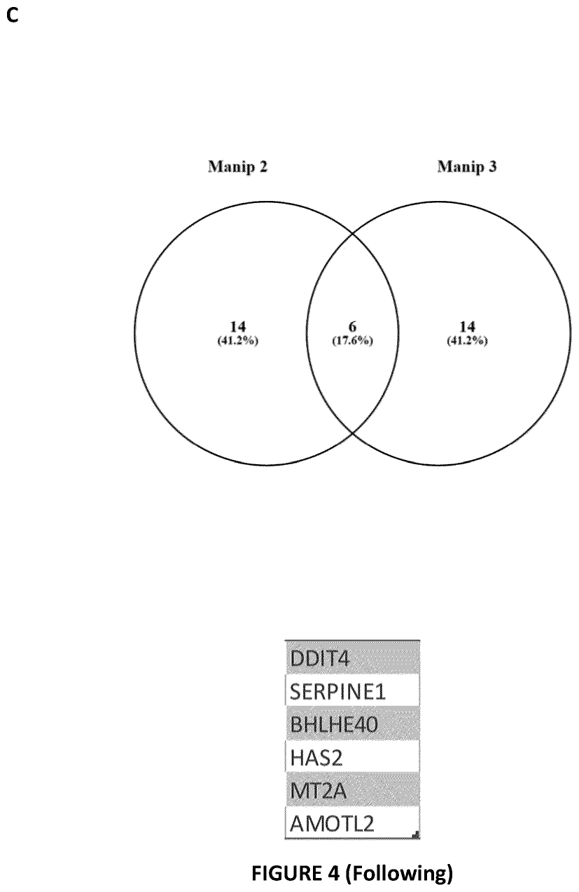

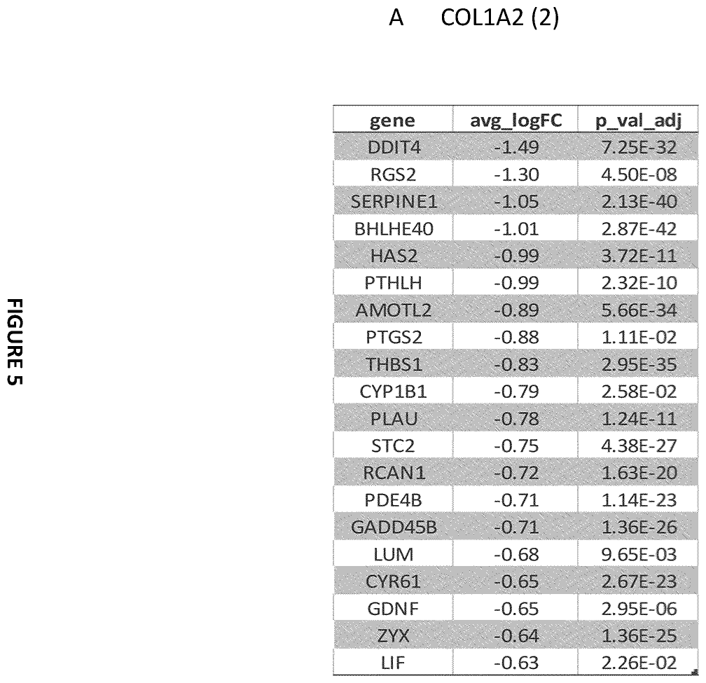

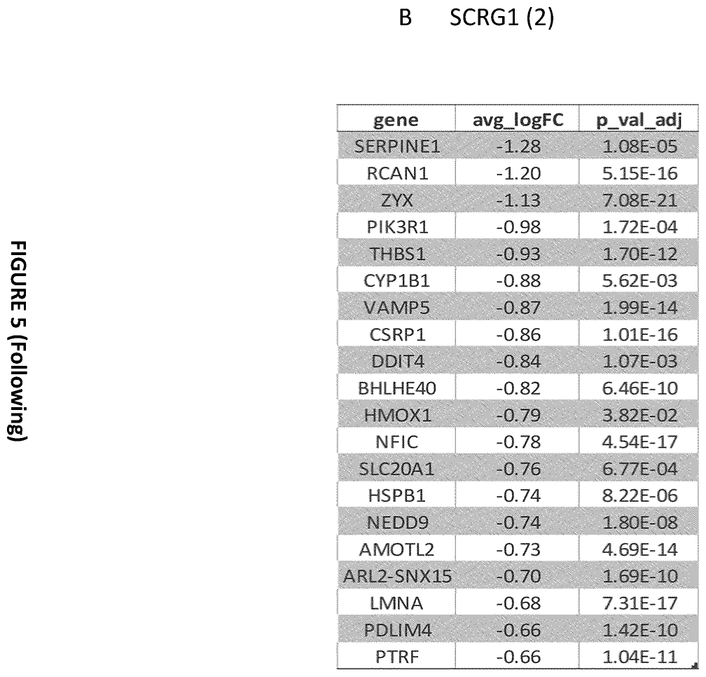

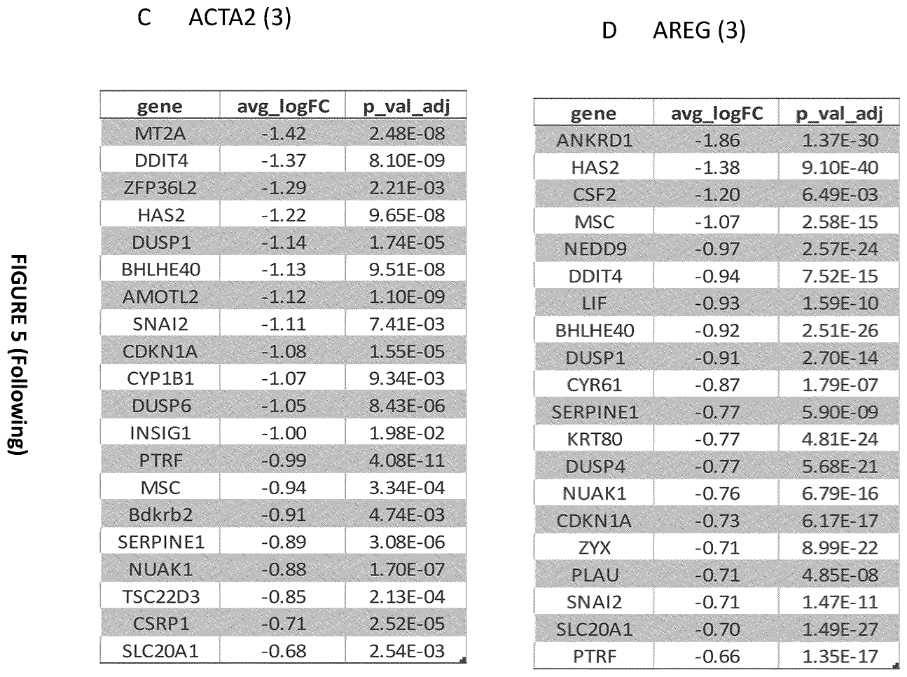

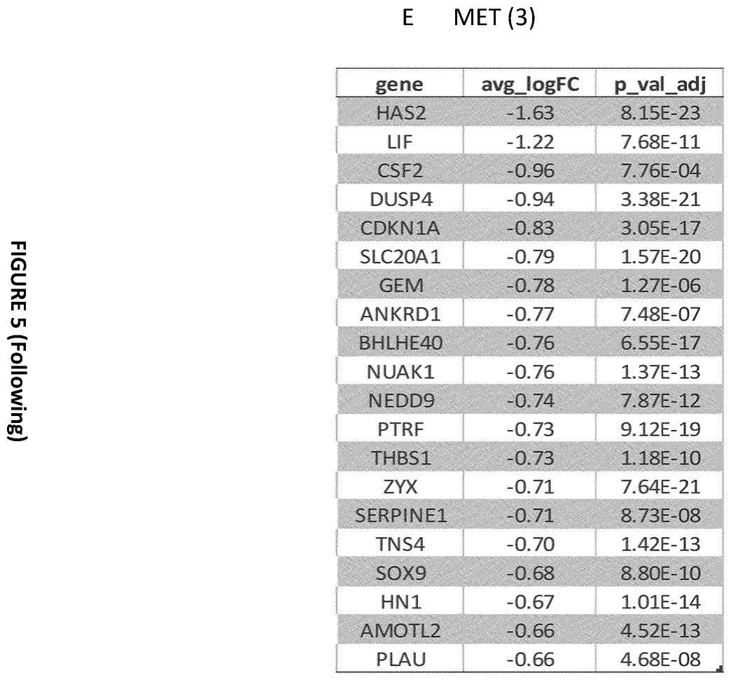

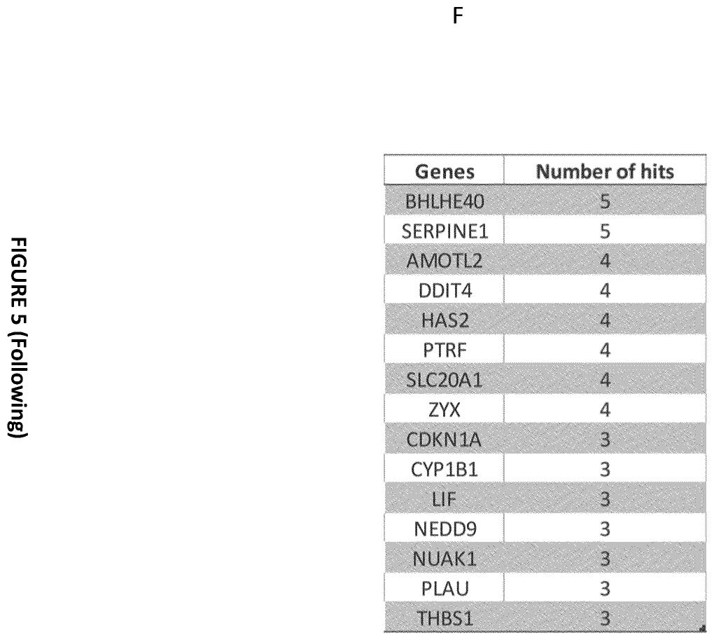

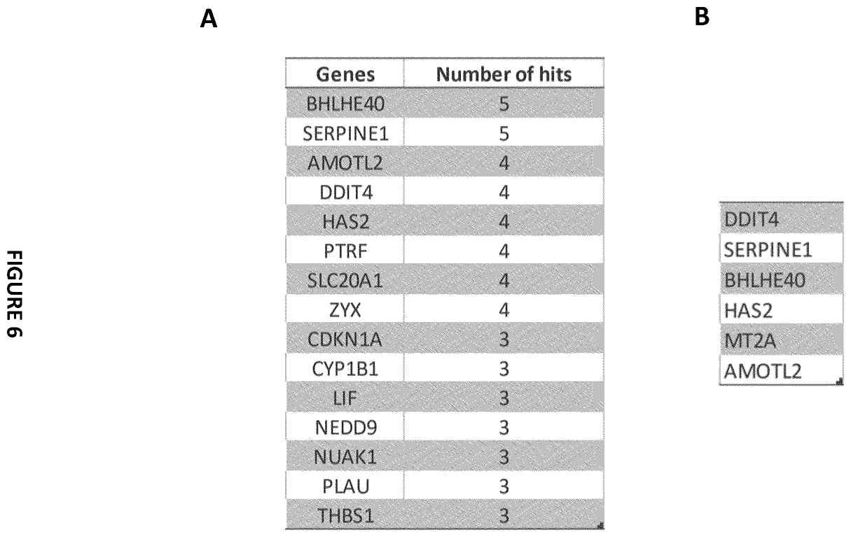

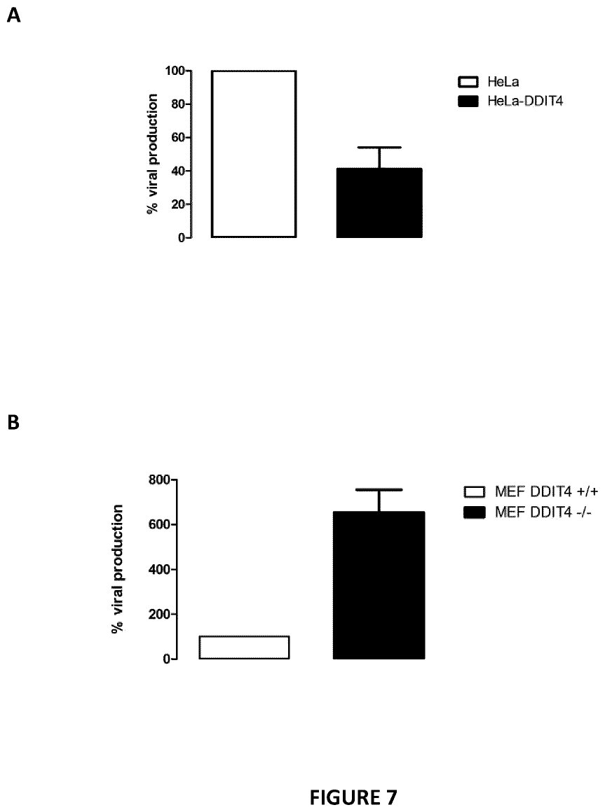

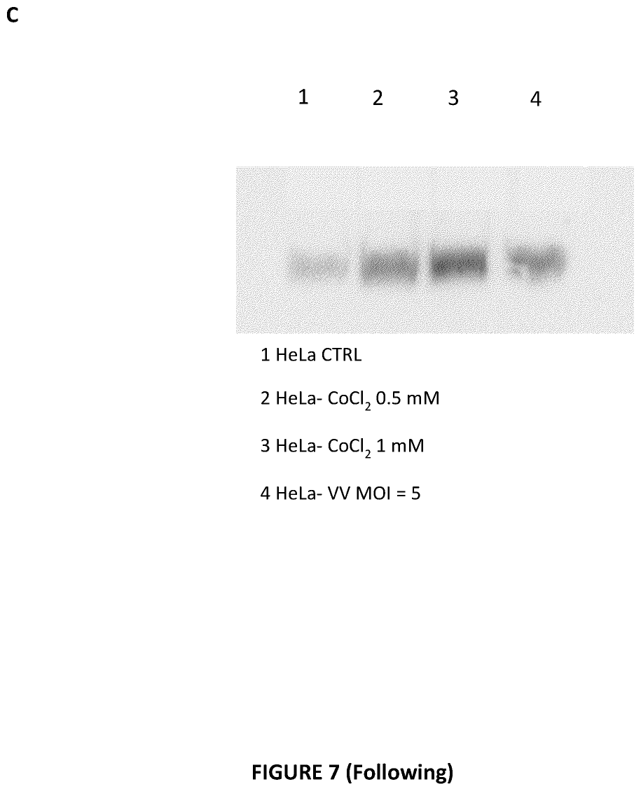

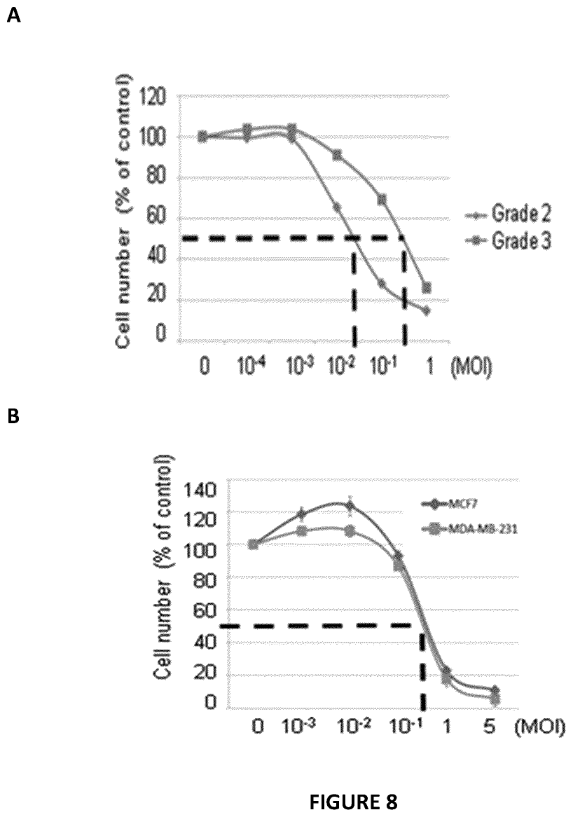

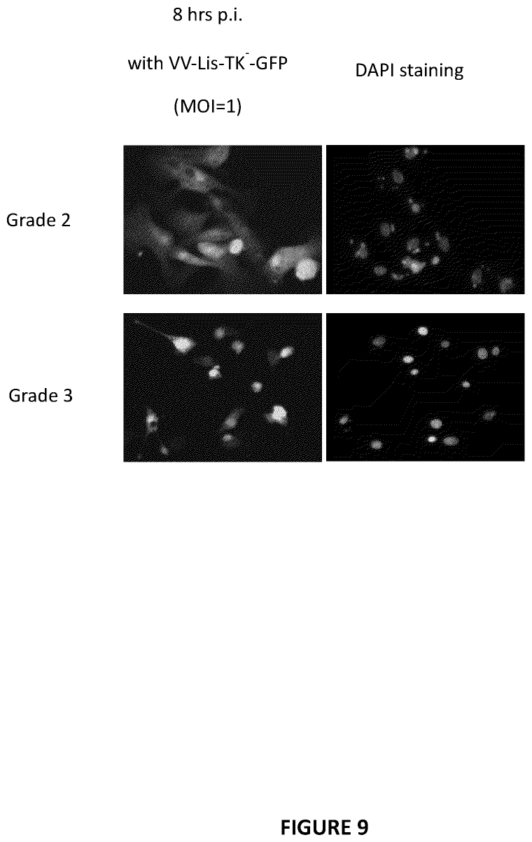

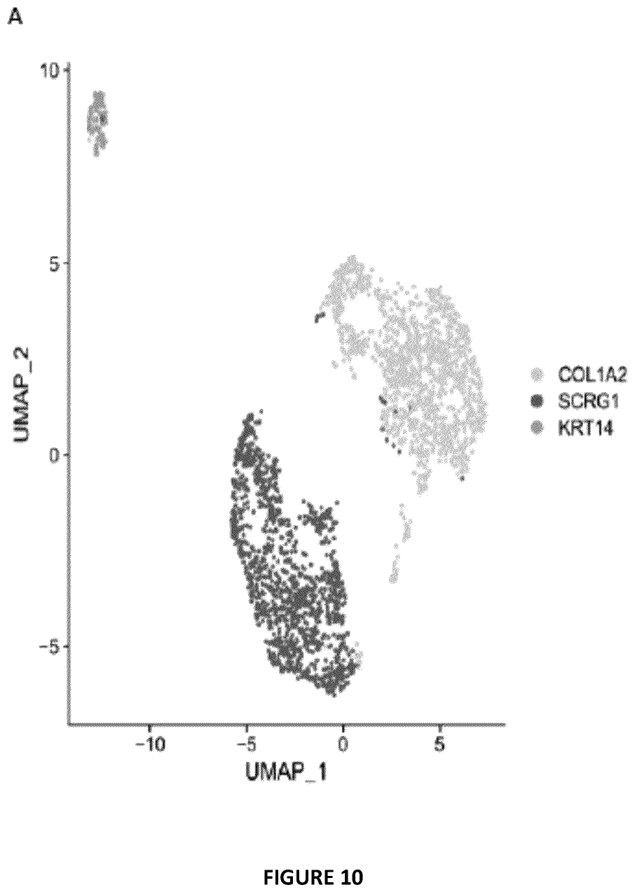

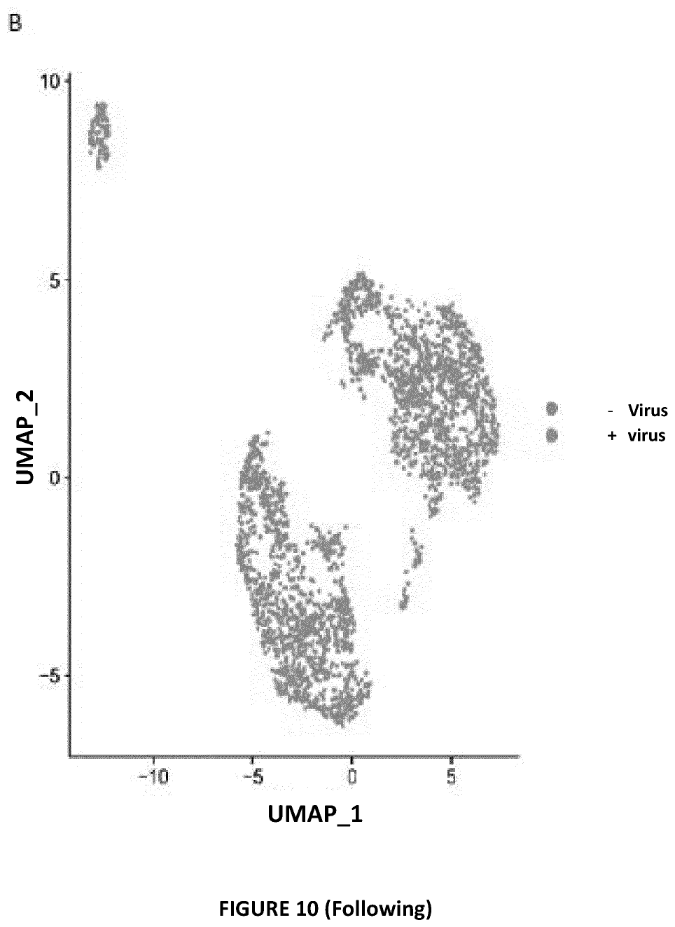

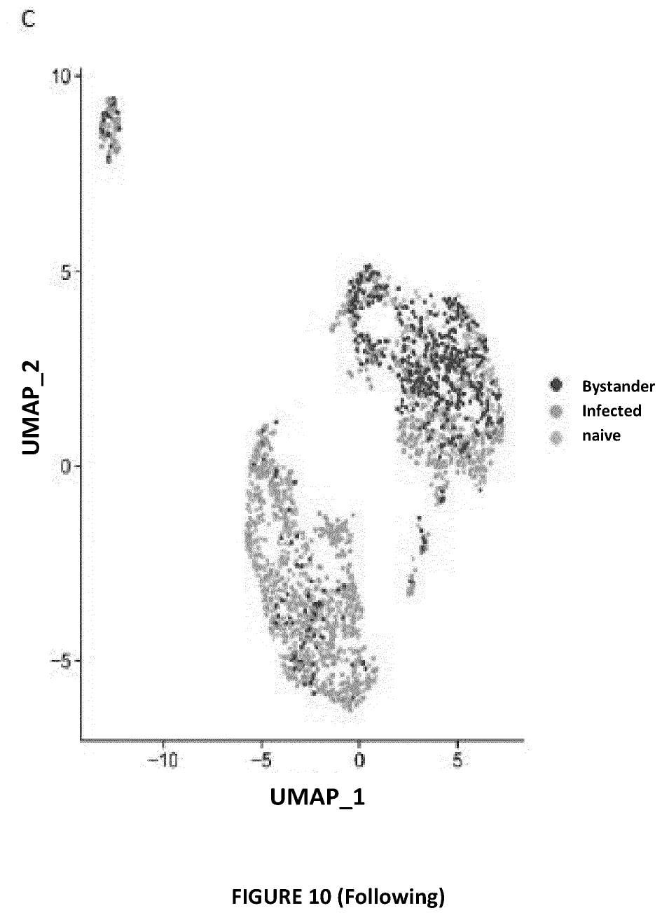

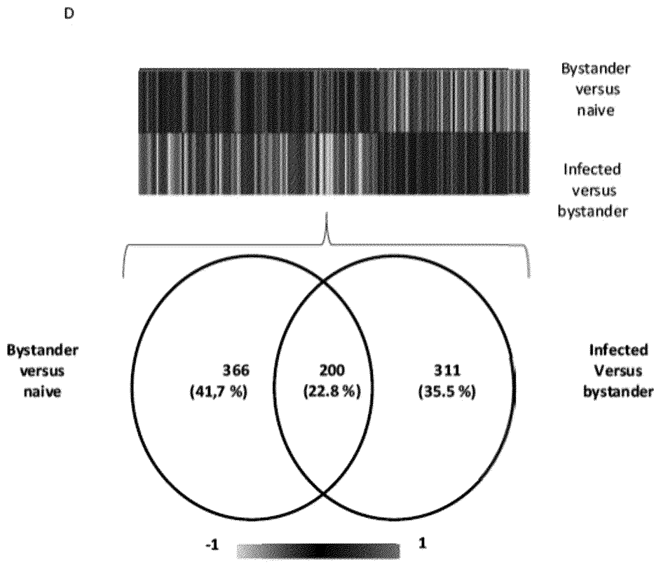

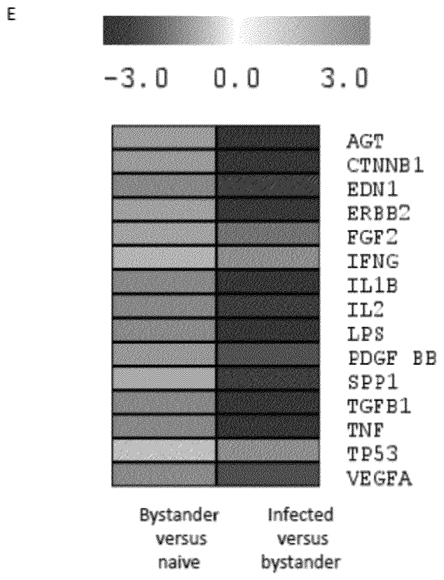

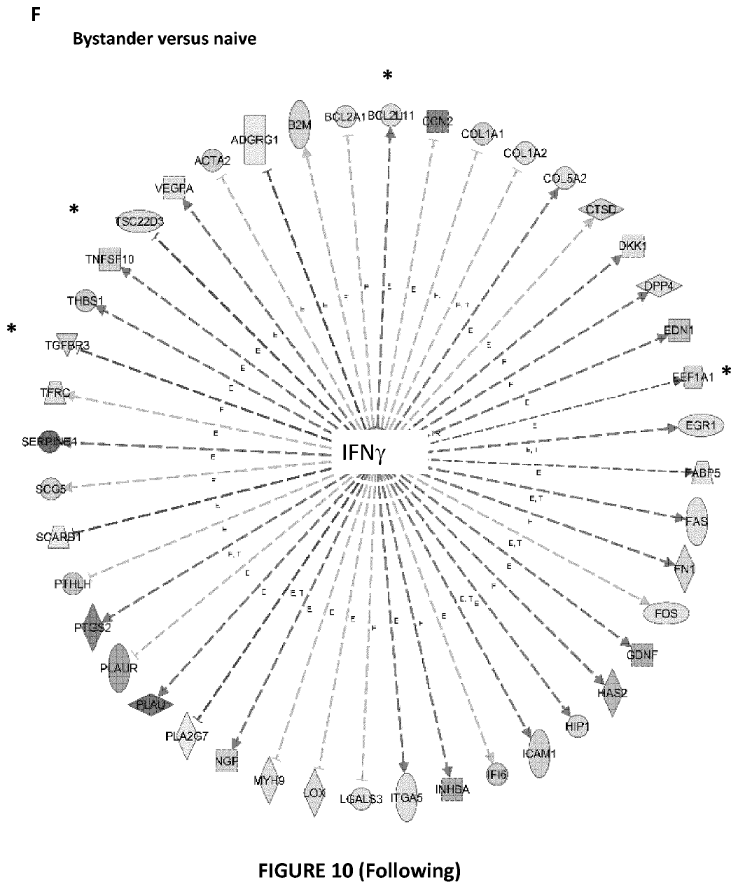

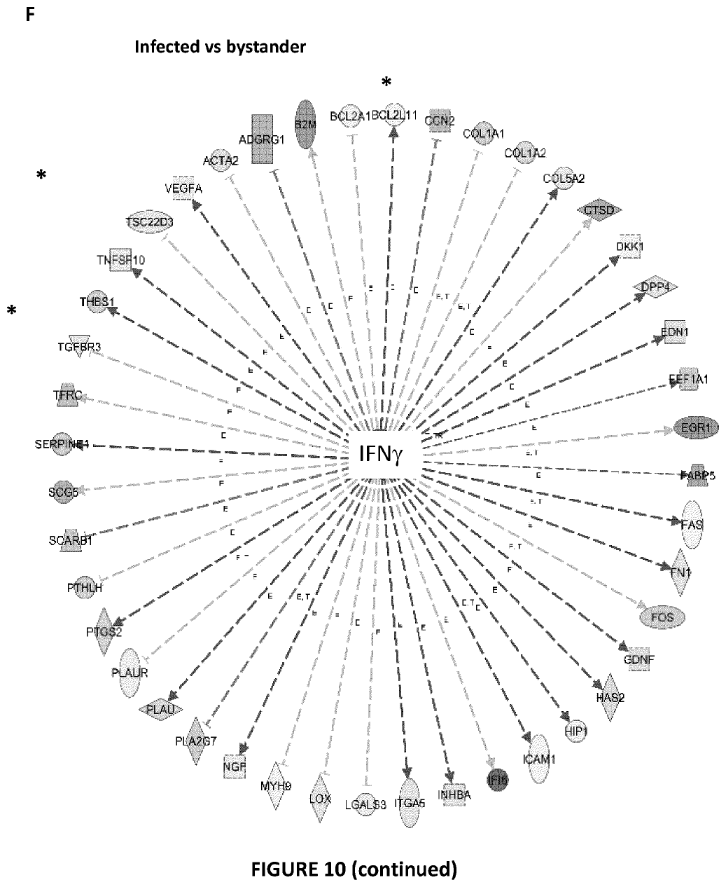

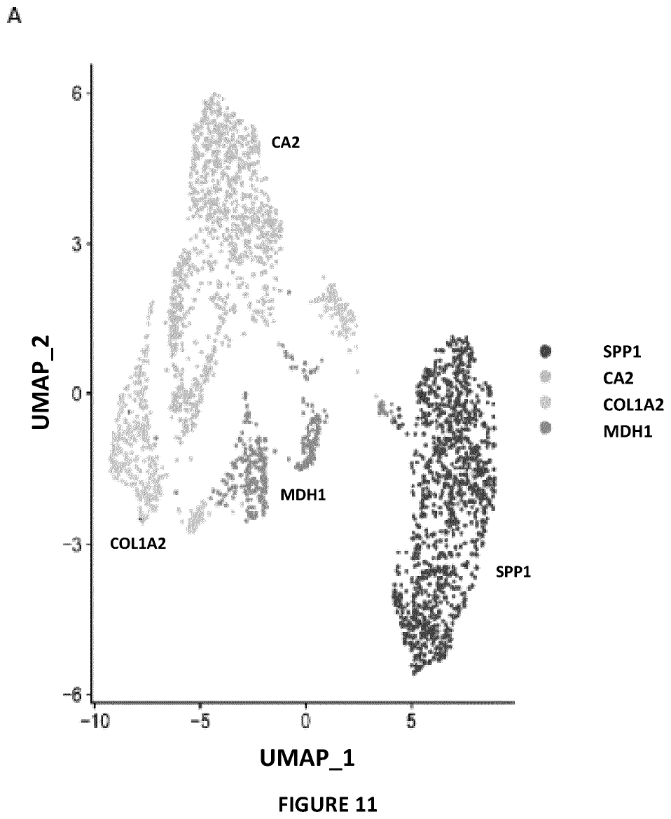

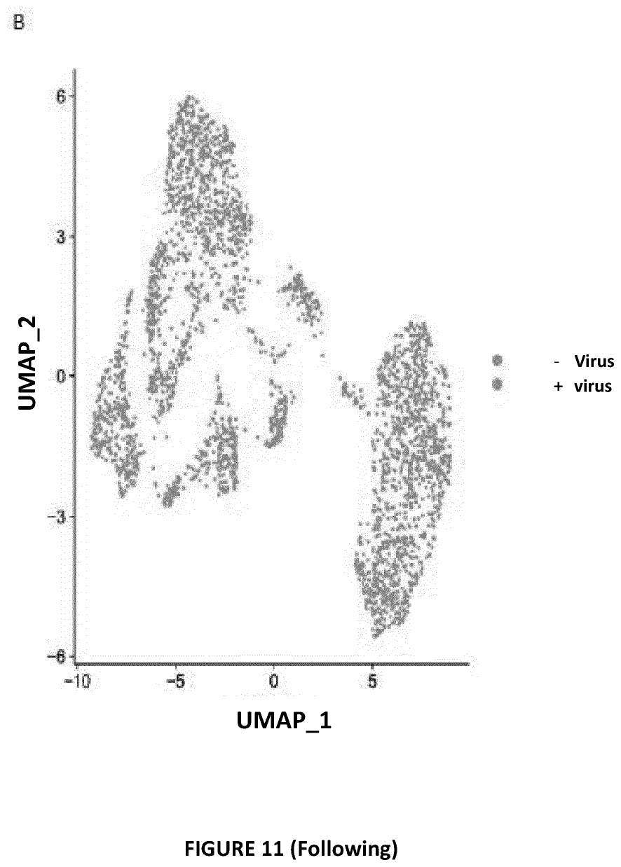

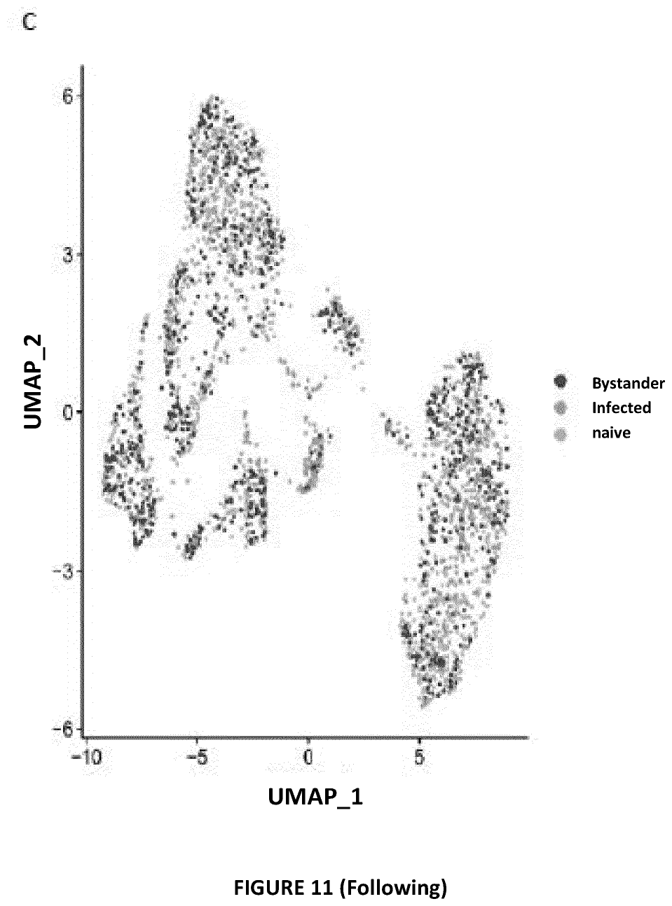

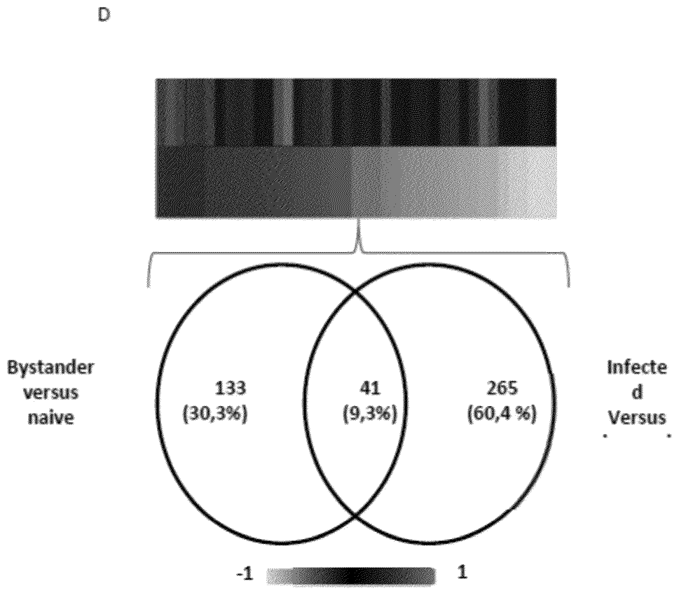

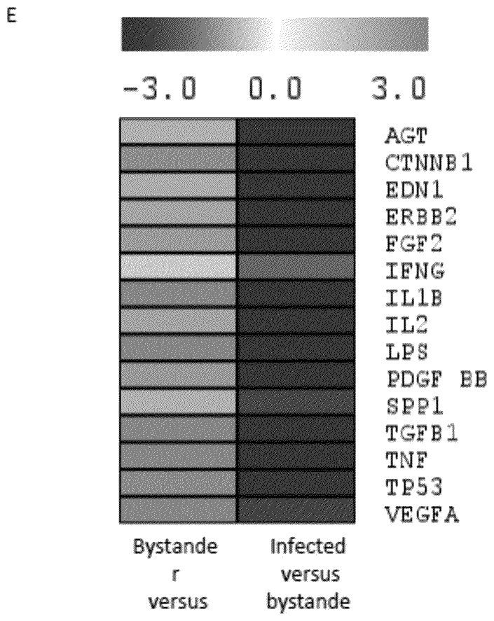

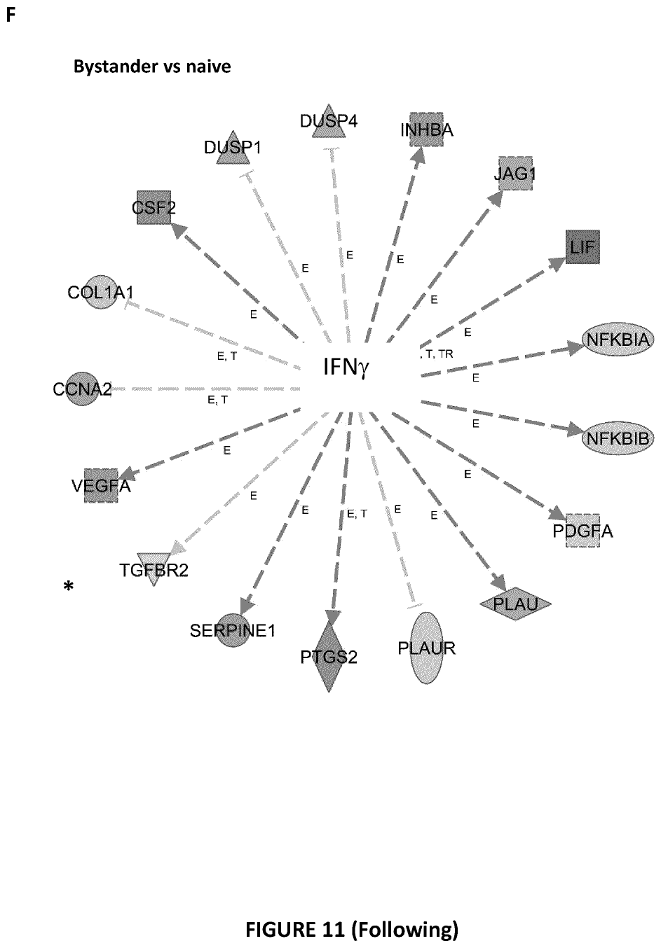

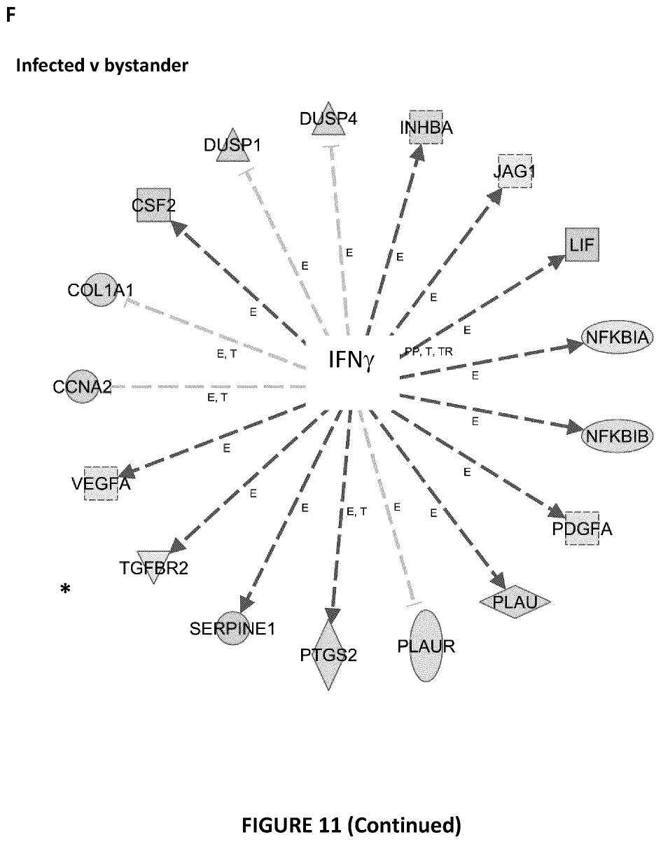

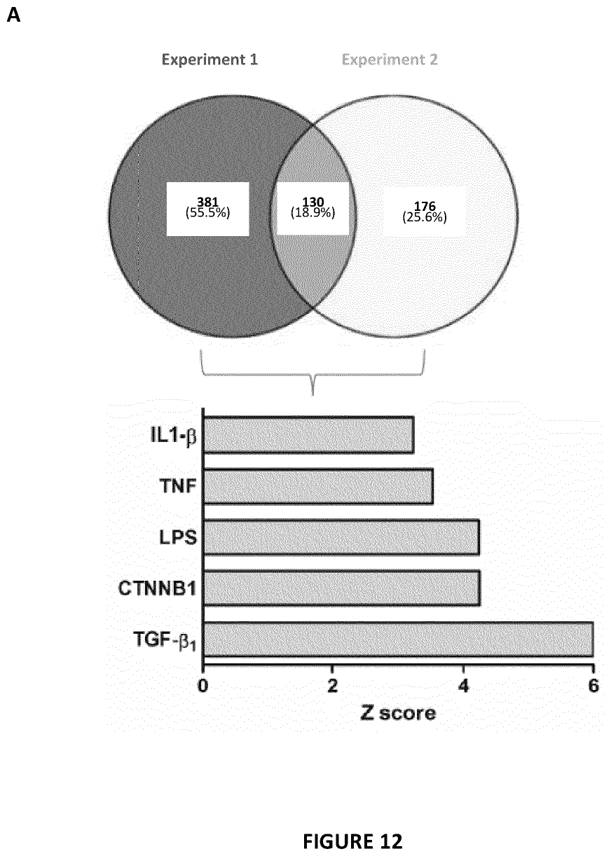

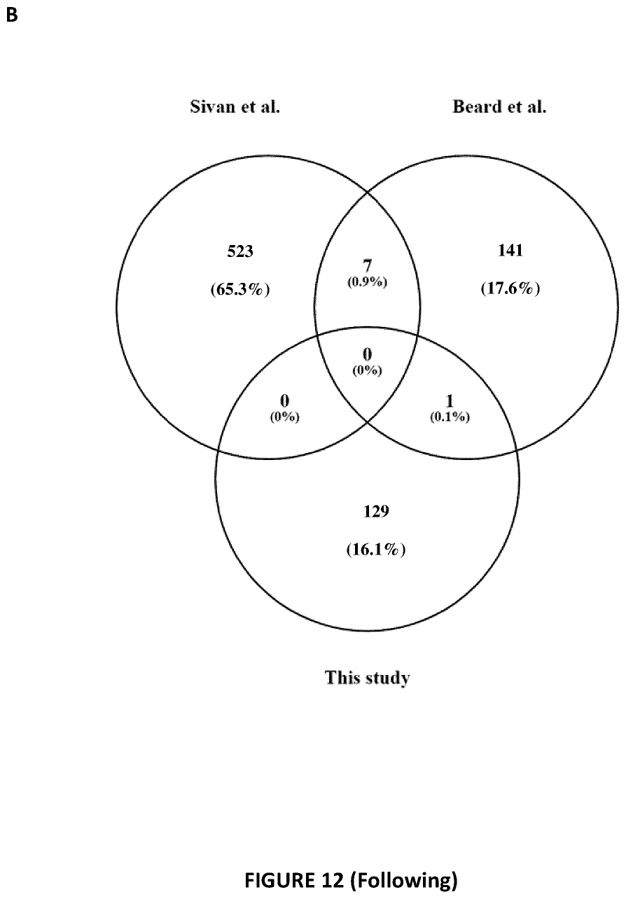

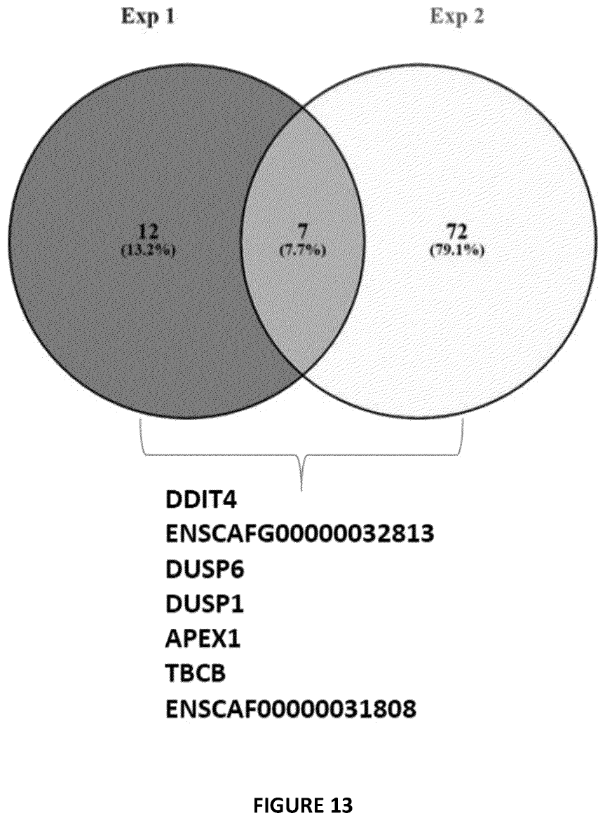

Cells from triple negative carcinomas cells (TNBC) or non-TNBC cells were infected with VV at different multiplicity of infection (MOI) and the numbers of cells remaining in the culture wells were monitored after four days. FIG. 8A presents an example of dose-response curves showing that non-TNBC cells are more sensitive to VV-mediated cell lysis than TNBC cells. The combined LD50 of experiments performed on all available samples (n=16 non-TNBC and n=6 for TNBC) are presented in FIG. 1A and show that non-TNBC cells are 28 times more sensitive to VV than TNBC cells. This observation is in sharp contrast to the situation observed in human established cell lines in which MCF7 cells (as a representative of the non-TNBC cells) and MDA-MB-231 cells (as a representative of TNBC cells) show an equivalent sensitivity to VV-induced cell lysis (FIG. 8B). The viral production was compared in canine TNBC and non-TNBC cells. Quantitative PCR to determine the number of viral genomes produced upon infection (FIG. 1B) and titration to determine the number of viral particles showed a reduced number of infectious viral particles produced upon infection of TNBC cells (FIG. 1C).

Replication as Opposed to Viral Infection/Early Stage of Viral Transcription is Mainly Affected in Canine TNBC Cells.

Infection and early-stages of viral transcription of a VV in which GFP expression is driven by an immediate-early VV promoter was examined on TNBC or non-TNBC cells. Count of the propidium iodide-positive and GFP-positive cells showed a statistically-significant, 5% difference in infection/early-stage of viral transcription between the two types of cells (FIG. 1D). Propidium iodide staining showed a classical nuclear labelling as well as cytosolic dots (FIG. 9). These structures are usually found in cells infected with VV and are often referred to as DNA factories or mininuclei (Cairns, 1960; Katsafanas and Moss, 2007). They are sites of viral DNA replication (Katsafanas and Moss, 2007). The percentage of mininuclei-positive cells in GFP-positive cells 8 hours after infection was 53% and 26.6% in non-TNBC and TNBC cells, respectively (FIG. 1E). In mininuclei-positive cells, an average of 3 and 1 mininuclei were found in the cytosol of non-TNBC and TNBC cells, respectively (FIG. 1F). Altogether, these data show that although a difference in the efficacy of infection/early stage of viral transcription can be detected in non-TNBC and TNBC cells, the main quantitative difference lies in the number of DNA factories, as in an average population, 6 times more viral DNA factories can be detected in non-TNBC compared to TNBC cells.

Upon infection, early viral genes are rapidly transcribed by a RNA-polymerase and factors packaged within the infectious particles (Yang et al., 2010). By contrast, the expression of intermediate- and late-viral genes requires de novo protein synthesis and viral replication in DNA factories (Yang et al., 2010). An implication of the results presented in FIG. 1 is that the expression of the early viral genes should be comparable in non-TNBC and TNBC, while the expression of intermediate and late genes should be impaired in TNBC. To assess this hypothesis, kinetics of expression of the early gene E9L and late gene A27L were performed on non-TNBC and TNBC. The expression of E9L is comparable in non-TNBC and TNBC cells 2 and 4 hours after infection and a difference in E9L expression is clearly visible 8 hours after infection (Data not shown). The late viral gene A27L was hardly detectable 2 and 4 hours after infection and its expression markedly increased 8 h post-infection in non-TNBC, while A27L expression remained low at this time-point. To confirm these data, kinetics of bulk RNA-Seq transcriptional analysis of non-TNBC versus TNBC cells infected with VV was performed using another pair of donors. FIG. 2 shows that the difference of expression of the whole early viral genes in non-TNBC versus TNBC cells is detectable and is statistically significant. However, this difference is much greater when whole intermediate and late viral gene expression is concerned. Altogether, these data suggest that, although infection/very viral early gene expression is lower in TNBC than in non-TNBC cells, the number of mininuclei, the replication of the virus and subsequent expression of the intermediate and late viral gene are quantitatively more affected.

Single-Cell RNA Sequencing to Dissect VV Infection of TNBC Cells: Impact of the Infection on Cellular Genes.

To characterize further the infection of TNBC cells by VV, inventors performed single-cell transcriptomic analysis. In these experiments, two independent TNBC primary cell cultures were either mock infected or infected with VV at a MOI of 5. Six hours later, the cells were trypsinized and subjected to the 10× Genomics single-cell protocol, followed by NG sequencing. FIGS. 3A and 3B shows that, in the two experiments performed, the number of cellular genes expressed decreases as the extent of viral genes expression increases. Furthermore, this decrease in the number of cellular genes expressed is more important in the subset of cells expressing the late viral genes (in red).

Differential Expression Analysis Using Naïve, Bystander and Infected Cells.

For each experiment, a t-SNE plot was created using the dataset obtained with the naïve cells (uninfected). The cells segregated in four (experiment 1) and seven (experiment 2) clusters (Data not shown). To pursue the analysis, inventors decided to exclude the cycling cells (providing excessive unnecessary information) as well as cells expressing viral genes and in which the number of cellular genes was below a threshold of 5%. New t-SNE plots were drawn and the cells segregated in three (experiment 1) and seven independent cellular clusters (experiments 2) (FIGS. 3C and D). Three distinct cellular populations were defined: naïve cells, defined as cells not exposed to VV; bystander cells, defined as cells exposed to the virus but expressing less than 1% of early viral genes; infected cells, defined as expressing more than 1% of early viral genes. Naïve, bystander and infected cells were localized onto the t-SNE plot (FIGS. 3E and 3F). The distribution showed that cells from these three populations were represented in each of the independent cellular clusters.

Identification of Genes Overrepresented in Bystander Versus Infected Cells.

Inventors hypothesized that genes with “antiviral” activities were overrepresented in the bystander population of cells and underrepresented in the infected population. A differential transcriptional analysis was therefore performed in these two cellular populations. A bulk analysis was performed and the top 20 genes differentially expressed are presented in FIGS. 4A and 4B.

Six differentially expressed genes are commons to the two experiments (FIG. 4C). Another analysis, in which individual clusters containing both bystander and infected cells was performed (FIG. 5). Out of the ten clusters obtained in the two experiments performed (FIG. 5), only 5 provided a list of genes for which the differential expression was statistically significant.

The top 15 genes overrepresented in the five remaining clusters are presented in FIG. 5F. The genes of these top 15 present in at least 3 clusters were selected and are listed.

The comparison of the bulk analysis and the cluster analysis provided a total of 15 genes as 5 of the 6 genes selected in the aggregated cluster analysis (FIG. 4) were also present in the list of selected genes in the “by cluster” analysis (FIG. 6B). In the light of inventors' hypothesis, these genes are candidates for a role as “antiviral genes”. Four genes were more particularly studied.

DDIT4 Exerts an Antiviral Activity.

Inventors examined the role of DDIT4 in VV replication.

Infection of HeLa cells overexpressing DDIT4 resulted in a 60% reduction in the production of infectious VV particles compared to control HeLa cells expressing GFP (FIG. 7A). Inversely, infection of mouse embryonic fibroblast (MEF) from DDIT4 knock out mice resulted in a six-time increase in the production of infectious VV particles compared to MEF isolated from wild-type mice (FIG. 7B). This quantitative difference between the gain and loss of function experiments is likely to be due to the fact that infection of parental HeLa cells with VV results in an induction of DDIT4 (FIG. 7C).

### DISCUSSION

In this experimental part, inventors demonstrate that oncolysis induced by VV is significantly less efficient in primary, high-grade canine mammary carcinoma than in equivalent cells obtained from lower grade tumors. This observation is in sharp contrast with the fact the same virus is equally efficient in established cell lines from differentiated/low-grade and in high-grade TNBC. Considering the close relationship between the human and canine pathologies (Queiroga F L et al.), it is tempting to attribute this difference in effectiveness of VV to the primary/low passage versus established cell lines status of the experimental models. The relevance of established cell lines as experimental systems to develop new therapeutic agents has largely been questioned in the past and the need for new preclinical models has been highlighted (Gillet J P et al.). Patient-derived xenografts have been proposed and are viewed as one of the most relevant modelling system in oncology (Williams J A. et al.). For a selected number of types of tumors that include breast cancer as well as osteosarcoma, lymphoma, melanoma, prostate cancer and soft tissue sarcoma, canine tumors recapitulate the features of human ones and resources from relevant canine tumors have been proposed as tools for the preclinical development of new cancer therapeutics (LeBlanc A K. et al.). One of these resources is very low passage primary cells grown in serum-free medium. Working with primary, low-passage cells has to date been hampered by the low numbers of cells available from biopsies. It is rare to collect more than 2-3 million carcinoma cells from one biopsy, and without amplification, this low number of cells restricts considerably the information that can be experimentally gathered. However, with the advent of single-cell transcriptomic, descriptive studies demonstrating whether a therapeutic agent is effective or not can be complemented with high-resolution molecular data. Inventors herein demonstrate for the first time that this information can lead to the identification of specific genes that affect the replication of VV. One of these genes is DNA damage inducible transcript 4 (DDIT4). DDIT4 is expressed in breast cancer and associated with a poor prognosis in various cancers that include breast cancers (Pinto J A et al., 2017). In high-grade, triple-negative breast cancers, DDIT4 is also associated with a poor prognostic in human patients (Pinto J A et al., 2016). DDIT4 has been associated with a worse prognostic in human patients with acute myeloid leukemia, glioblastoma multiforme, colon, skin and lung cancers in addition to breast cancer (Pinto et al., 2017).

Biochemically, DDIT4 has been described largely as a negative regulator of the mTOR signalling pathway (Ben Sahra et al., 2011; Brugarolas et al., 2004). Rapamycin, a pharmacological inhibitor of the mTOR signaling pathway, has also been described to reduce the virus yield upon VV infection (Soares et al., 2009). A likely mechanism was that mTOR activation resulted in the phosphorylation of 4E-BP, which in turn release the translation factor elF4E, the component of el4F that binds to the 5′-cap structure of mRNA and promotes translation (Kapp and Lorsch, 2004; Soares et al., 2009). Upon VV infection, the factor elF4E has been reported to be redistributed in cavities present within viral factories (Katsafanas and Moss, 2007; Walsh et al., 2008) where viral translation can proceed. It is therefore tempting to hypothesize that DDIT4, by inhibiting the mTOR signaling pathway, reduces the amount of elF4E available for viral translation.

The identification of cellular genes promoting or restricting vaccinia virus infectivity/replication has been studied using hypothesis-driven approaches (Caceres A et al., Ibrahim N. et al., Guerra S. et al.) or high-throughput RNA interference screens (Mercer J. et al., 2012; Sivan G. et al., 2013; Beard P M. et al., 2014; Sivan G. et al., 2013; Sivan G. et al., 2015) and in this context single-cell transcriptomic increases the arsenal of experimental tools available. If DDIT4 expression can reduce viral yield, inventors believe that other cellular genes are involved and may act in concert to establish the relative refractory state observed in TNBC cells (FIG. 1). A list of fifteen genes is presented in FIG. 6. By comparison with genes identified as potential anti-viral genes in high throughput RNAi screens, only one gene (SERPINE1) was found in common with the study of Beard et al. (Beard et al., 2014). This low overlap is hardly surprising considering that both virus and cells are different from these screens. Nevertheless, it highlights the complexity of the interactions of vaccinia virus with host cells. Finally, in inventors' study, the number of 15 genes could be increased by reducing the stringency of the selection criteria. For example, DUSP1 is listed in two of the five top 20 genes in the cluster analysis presented in FIG. 5.

The utilization of single cell transcriptomic in the field of infectious diseases has already been used. For example, the extreme heterogeneity of influenza virus infection (Russell. et al., 2018) and study of influenza infection of mouse lungs in vivo have been reported (Steuerman et al., 2018). But, to inventors' knowledge, it has never been applied to the study of vaccinia virus infection. First, the present study confirms the transcriptional shut-down of cellular genes. In inventors' dataset, this shut-down is correlated to the extent of viral gene expression (FIG. 3). A unique feature of single-cell transcriptomic analysis is the possibility of dissecting different populations of cells that have been in contact with the virus. Cells expressing intermediate and late viral genes express a low number of cellular genes. Their inclusion in the bioinformatics analysis did not provide any particular information in inventors' quest to identify antiviral genes.

By contrast, bystander cells and cells expressing early-viral genes (and still expressing more than 50% of cellular genes) provide a unique source of information. Comparison of bystander and naïve cells showed, amongst others, an activation of the pathways regulated by TGFb1, TNF, NFkB, LPS and IL1b in bystander cells. These activations appear to be the results of the combined action of the pathogen-associated molecular pattern of the virus and of autocrine factors secreted by infected and dying cells. Inversely, an inhibition of these pathways can be observed when infected and bystander cells are compared. Inventors herein demonstrate that these inhibitions result from the expression of viral genes that counteract the cellular responses.

### Example 2

Differential Expression Analysis Using Naïve, Bystander and Infected Cells

To characterize further the infection of TNBC cells by VV, inventors performed single-cell transcriptomic analysis. In these experiments, two independent TNBC primary cell cultures were either mock infected or infected with VV at a MOI of 5. Six hours later, the cells were trypsinized and subjected to the 10× Genomics single-cell protocol, followed by sequencing. For the 2 experiments, a standard statistical analysis using Seurat v3 was performed using cells with a percentage of mitochondrial genes below 25%. On the UMAP plots produced, the cells segregated in three (experiment 1, FIG. 10A) and four (experiment 2, FIG. 11A) clusters. These clusters contained both control cells and cells exposed to the virus (FIGS. 10B and 11B). Three distinct cellular populations were distinguished among the different clusters: naïve cells, defined as cells not exposed to VV; bystander cells, defined as cells exposed to the virus but expressing less than 0.01% of early viral genes; infected cells, defined as expressing more than 0.01% of early viral genes. Naïve, bystander and infected cells were localized onto the UMAP plot (FIGS. 10C and 11C). The distribution indicated that the clusters showing the higher proportion of bystander cells in the two experiments are the “COL1A2” clusters. The relative proportion of the three subpopulations (naïve, bystander, infected) in all the clusters are presented in the following Table 1.

TABLE 1

| | | | | |
|---|---|---|---|---|
| | | | | Ratio |
| Clusters | Naive | Bystander | Infected | bystander/infected |

Experiment 1

| | | | | |
|---|---|---|---|---|
| COL1A2 | 510 | 418 | 271 | 1.54 |
| SCRG1 | 602 | 75 | 437 | 0.17 |
| KRT14 | 42 | 20 | 33 | 0.61 |

Experiment 2

| | | | | |
|---|---|---|---|---|
| SPP1 | 576 | 262 | 193 | 1.36 |
| CA2 | 503 | 258 | 188 | 1.37 |
| COL1A2 | 256 | 156 | 55 | 2.84 |
| MDH1 | 136 | 73 | 107 | 0.68 |

Assuming that a higher proportion of bystander cell within a cluster is associated with an increased refractoriness to the virus, we looked upstream regulators associated with the two “COL1A2” clusters using Ingenuity Pathway Analysis™ (IPA) analysis. The transcriptomic signatures of the cells show, for the two clusters, a pattern highly consistent with “TGF-β” as a major upstream regulator. To describe the molecular events associated with viral infection, a differential expression analysis was performed between bystander and naïve cells and between infected and bystander cells. The whole dataset is presented in Table 2A and B (experiment 1) and Table 3A and B (experiment 2).

TABLE 2A

| Genes | avg_1ogFC | p_val_adj |
|---|---|---|
| SERPINE1 | 2.270134566 | 1.64E−124 |
| PLAU | 1.938321329 | 3.33E−101 |
| TF | 1.615323222 | 8.66E−70 |
| CYP1B1 | 1.605188409 | 3.59E−52 |
| PTGS2 | 1.601522321 | 2.60E−53 |
| CTGF | 1.594792721 | 2.67E−101 |
| GADD45B | 1.575014248 | 2.41E−111 |
| PMEPA1 | 1.368238395 | 1.77E−138 |
| RGS2 | 1.33586132 | 3.82E−30 |
| LIF | 1.185820503 | 2.35E−68 |
| ENSCAFG00000007045 | 1.127510697 | 4.59E−56 |
| PLAUR | 1.080355294 | 2.92E−83 |
| BHLHE40 | 0.987427044 | 2.44E−75 |
| STC2 | 0.973849543 | 1.62E−60 |
| GDNF | 0.951146608 | 1.94E−65 |
| INHBA | 0.938581446 | 1.09E−42 |
| FSTL3 | 0.915068598 | 1.25E−68 |
| ALAS1 | 0.910462856 | 9.86E−46 |
| DDX58 | 0.907727781 | 7.51E−05 |
| SNAI1 | 0.880863852 | 3.42E−59 |
| HAS2 | 0.831263218 | 1.42E−38 |
| CYR61 | 0.822041938 | 1.81E−55 |
| VCAN | 0.791744247 | 3.50E−53 |
| HBEGF | 0.743209509 | 3.29E−31 |
| LTBP1 | 0.726556801 | 6.54E−43 |
| UGDH | 0.723736501 | 2.68E−54 |
| SKIL | 0.722523271 | 1.13E−68 |
| ENSCAFG00000032813 | 0.69050212 | 2.99E−22 |
| CYP26B1 | 0.66026285 | 7.51E−06 |
| EDN1 | 0.659509753 | 1.23E−11 |
| FST | 0.649885638 | 1.53E−06 |
| RASL11A | 0.643700697 | 1 |
| PTHLH | 0.632177886 | 5.00E−13 |
| TFPI2 | 0.631465532 | 3.09E−31 |
| BAZ1A | 0.625318472 | 3.17E−59 |
| LRRC8C | 0.613489029 | 3.03E−43 |
| PDPN | 0.612788854 | 1.17E−25 |
| ISG20 | 0.610179996 | 1.71E−28 |
| ACTN1 | 0.607997286 | 7.22E−54 |
| THBS1 | 0.572688209 | 1.13E−34 |
| ENSCAFG00000015625 | 0.569353099 | 4.40E−22 |
| ENSCAFG00000002015 | 0.566931371 | 2.05E−46 |
| CYCS | 0.565808469 | 3.55E−63 |
| PDE4B | 0.564477965 | 1.51E−27 |
| COL5A1 | 0.55491057 | 6.15E−27 |
| PDLIM5 | 0.55338401 | 5.05E−42 |
| ELOVL4 | 0.541004424 | 8.53E−32 |
| ICAM1 | 0.540189379 | 1.40E−31 |
| FOXP4 | 0.538976974 | 5.14E−38 |
| NGF | 0.536034633 | 7.86E−25 |
| RBMS1 | 0.520889735 | 1.39E−56 |
| ACTA2 | 0.51835637 | 4.77E−05 |
| ENSCAFG00000033998 | 0.510076931 | 2.01E−21 |
| ADSS | 0.508717264 | 2.27E−29 |
| WISP1 | 0.505862364 | 1.47E−14 |
| ELK3 | 0.505306706 | 4.15E−28 |
| MAP1B | 0.501889012 | 3.96E−29 |
| S100A2 | 0.501136569 | 1 |
| MARCH3 | 0.495772749 | 1.36E−25 |
| NF2 | 0.494490521 | 4.59E−16 |
| ENSCAFG00000003758 | 0.49399392 | 1.14E−30 |
| SNRNP48 | 0.489452297 | 3.82E−37 |
| NIPAL1 | 0.488938551 | 9.16E−25 |
| TNFSF10 | 0.487114772 | 1.26E−27 |
| MT2A | 0.484966088 | 9.09E−27 |
| MEDAG | 0.483035018 | 5.29E−14 |
| COL1A1 | 0.481300099 | 2.93E−14 |
| IFRD1 | 0.47940483 | 1.85E−26 |
| DNAJB4 | 0.478466459 | 2.86E−25 |
| KCTD10 | 0.472668513 | 7.71E−34 |
| RCAN1 | 0.467451478 | 2.95E−19 |
| PALLD | 0.46704451 | 3.09E−25 |
| FZD2 | 0.461441264 | 3.70E−42 |
| KIF5B | 0.452934638 | 9.92E−51 |
| FN1 | 0.451706657 | 7.67E−21 |
| ENSCAFG00000006436 | 0.450184308 | 9.50E−28 |
| GLRX | 0.447673859 | 7.04E−08 |
| VPS37A | 0.44658027 | 6.47E−31 |
| SLC4A7 | 0.44656017 | 8.43E−31 |
| ENSCAFG00000037735 | 0.44491503 | 9.27E−20 |
| NEK7 | 0.440671399 | 1.16E−23 |
| ENSCAFG00000024171 | 0.439808333 | 2.72E−13 |
| PLOD2 | 0.435799011 | 5.58E−32 |
| SSX2IP | 0.433433068 | 5.40E−22 |
| HIP1 | 0.430291869 | 2.35E−28 |
| ELL2 | 0.429880706 | 4.21E−26 |
| PTK7 | 0.428408906 | 2.31E−26 |
| ANO6 | 0.42819938 | 6.54E−27 |
| ETS1 | 0.42651363 | 1.16E−23 |
| COL8A1 | 0.425783098 | 1.12E−05 |
| IQCJ-SCHIP1 | 0.421507132 | 2.17E−15 |
| LOX | 0.421443873 | 1.73E−18 |
| SH3BP5L | 0.420108354 | 8.54E−23 |
| RAI14 | 0.418838543 | 5.16E−24 |
| SNX10 | 0.416905955 | 2.20E−24 |
| ADAM19 | 0.416225598 | 7.29E−18 |
| COL12A1 | 0.415635917 | 3.05E−22 |
| HSPH1 | 0.412816956 | 6.08E−27 |
| SRPX2 | 0.412666738 | 1.53E−24 |
| DLC1 | 0.411009682 | 2.22E−19 |
| ALDH18A1 | 0.410604989 | 6.49E−33 |
| AMOTL2 | 0.410370891 | 1.12E−18 |
| SDC4 | 0.406600535 | 1.73E−09 |
| PFKFB3 | 0.403011464 | 1 |
| NRP2 | 0.402252102 | 9.73E−24 |
| ENSCAFG00000000345 | 0.398102466 | 6.59E−26 |
| CRABP2 | 0.396979592 | 2.93E−14 |
| GORAB | 0.396186256 | 1.23E−15 |
| TPM2 | 0.394423601 | 0.0010039 |
| LUM | 0.392747857 | 2.43E−05 |
| ENSCAFG00000013979 | 0.388719855 | 3.97E−14 |
| SPHK1 | 0.38692647 | 4.99E−28 |
| IFIH1 | 0.38656085 | 7.45E−06 |
| COL3A1 | 0.383267542 | 4.72E−13 |
| BGN | 0.383210386 | 5.66E−14 |
| PICALM | 0.382048887 | 1.62E−29 |
| RALA | 0.377466022 | 4.13E−22 |
| ITGA5 | 0.372426688 | 0.00022458 |
| ZNFX1 | 0.371873777 | 2.03E−23 |
| ENSCAFG00000036684 | 0.371392695 | 1.83E−17 |
| MAP7D1 | 0.367099126 | 8.37E−18 |
| SRM | 0.366802221 | 1.71E−19 |
| BCL2L11 | 0.366623544 | 1.54E−14 |
| AEN | 0.366556769 | 4.58E−09 |
| MYH9 | 0.365525825 | 1.82E−23 |
| YIF1B | 0.364924894 | 5.58E−20 |
| NFKBIA | 0.363974328 | 0.00098373 |
| PHF20L1 | 0.363486584 | 5.95E−20 |
| PDZRN3 | 0.361763681 | 3.68E−24 |
| BLOC1S2 | 0.360110531 | 8.67E−25 |
| GTF2F1 | 0.359984498 | 7.39E−18 |
| PTGES | 0.354877191 | 1 |
| SPOP | 0.354769454 | 3.71E−17 |
| BCAR1 | 0.354670991 | 5.43E−18 |
| GFPT2 | 0.353801801 | 3.84E−08 |
| CKAP4 | 0.353447818 | 6.34E−26 |
| ENSCAFG00000031852 | 0.351467487 | 9.04E−27 |
| TUBA4A | 0.351136866 | 3.09E−12 |
| PNPLA2 | 0.351032625 | 5.69E−18 |
| CTNNB1 | 0.348949126 | 7.58E−35 |
| FAS | 0.348560418 | 7.60E−17 |
| SYNC | 0.346548987 | 2.88E−07 |
| MMD | 0.345335146 | 1.13E−15 |
| TCF4 | 0.34342355 | 2.19E−13 |
| TCIRG1 | 0.341695915 | 1.25E−15 |
| IBTK | 0.341267978 | 1.95E−18 |
| GOLT1B | 0.340457605 | 3.76E−21 |
| SNRNP40 | 0.34009679 | 1.00E−17 |
| COL1A2 | 0.339411575 | 2.05E−10 |
| HAPLN3 | 0.33610249 | 5.47E−13 |
| SACS | 0.33507603 | 4.06E−13 |
| PINX1 | 0.334967829 | 2.96E−13 |
| CDK8 | 0.334822383 | 1.34E−18 |
| ENSCAFG00000011482 | 0.332844714 | 2.69E−14 |
| UBE2J1 | 0.332459 | 8.26E−16 |
| S100A4 | 0.331511753 | 4.16E−05 |
| STARD13 | 0.330863114 | 7.13E−12 |
| SRSF7 | 0.330639938 | 2.33E−16 |
| MPP6 | 0.330320089 | 1.35E−13 |
| STK38L | 0.330194066 | 1.22E−11 |
| VEGFA | 0.326552564 | 7.11E−07 |
| GNL3 | 0.326489092 | 1.78E−15 |
| COL5A2 | 0.325283785 | 1.90E−16 |
| PID1 | 0.324789537 | 5.20E−07 |
| NUAK1 | 0.322608179 | 7.38E−12 |
| LMCD1 | 0.322505239 | 0.00031673 |
| LPCAT2 | 0.321883143 | 2.06E−12 |
| THBS2 | 0.321515274 | 9.22E−06 |
| ENSCAFG00000037410 | 0.321415081 | 1.18E−16 |
| ATP13A3 | 0.320744061 | 8.61E−15 |
| ANKRD10 | 0.320687221 | 2.04E−14 |
| TMEM2 | 0.320271599 | 6.72E−11 |
| ENSCAFG00000018440 | 0.318205368 | 3.56E−12 |
| REL | 0.316388368 | 3.30E−17 |
| SNAI2 | 0.316301052 | 1 |
| ENSCAFG00000001840 | 0.315952691 | 6.09E−13 |
| TXNRD1 | 0.314990868 | 2.15E−18 |
| RIC1 | 0.31260878 | 2.06E−14 |
| SMC5 | 0.312166654 | 8.07E−13 |
| NDUFAF4 | 0.312125069 | 1.97E−13 |
| NOP58 | 0.312027948 | 1.77E−09 |
| CSRP1 | 0.311483153 | 1.84E−23 |
| TMEM41B | 0.311216589 | 2.61E−12 |
| ENSCAFG00000017326 | 0.309316441 | 0.65348525 |
| NUDC | 0.308853972 | 1.66E−19 |
| STAM2 | 0.308195198 | 4.20E−20 |
| ENSCAFG00000001448 | 0.307218421 | 4.61E−30 |
| MYLK | 0.307130976 | 2.61E−09 |
| ACVR1 | 0.30491048 | 5.65E−13 |
| RNF6 | 0.304133178 | 1.63E−16 |
| CCT4 | 0.303922731 | 2.48E−17 |
| FAM49B | 0.302795732 | 1.45E−13 |
| TKT | 0.302581621 | 8.90E−06 |
| PCSK1 | 0.302052584 | 1.88E−31 |
| SMG7 | 0.301762461 | 1.82E−12 |
| MRPS31 | 0.301222195 | 6.87E−19 |
| TUBA1C | 0.300991012 | 2.83E−10 |
| UBE2S | 0.300842341 | 1.06E−13 |
| NUDCD1 | 0.29796364 | 2.49E−14 |
| CRLF3 | 0.297570697 | 9.93E−15 |
| TWF1 | 0.296148932 | 6.20E−16 |
| DDX60 | 0.295595225 | 1 |
| GTF2H1 | 0.29497148 | 3.45E−15 |
| FAM126A | 0.294401112 | 3.95E−12 |
| PNPLA8 | 0.292016173 | 1.55E−15 |
| FAM20C | 0.291791458 | 2.00E−09 |
| MT1 | 0.290657433 | 0.14826836 |
| DNAJB9 | 0.289623435 | 2.82E−12 |
| ENSCAFG00000029851 | 0.289545577 | 3.15E−14 |
| PRPF38B | 0.288814072 | 1.03E−13 |
| RPS6KC1 | 0.288104439 | 1.19E−14 |
| DKK1 | 0.288032443 | 3.79E−07 |
| ENSCAFG00000030449 | 0.285576385 | 5.59E−07 |
| DPP4 | 0.285457297 | 2.29E−11 |
| FLNB | 0.284752033 | 1.74E−16 |
| PLS3 | 0.284373241 | 5.79E−12 |
| CLCF1 | 0.284081513 | 4.42E−24 |
| ATP6V1H | 0.280867753 | 4.10E−12 |
| GPATCH4 | 0.280788015 | 3.35E−09 |
| ENSCAFG00000007306 | 0.279060899 | 1.93E−13 |
| TMF1 | 0.278899959 | 4.01E−12 |
| MAOA | 0.277829558 | 1.24E−11 |
| NAV3 | 0.277094704 | 1.59E−10 |
| SOD2 | 0.276366887 | 1.30E−12 |
| EMD | 0.276004674 | 1.41E−10 |
| ANKRD37 | 0.275977979 | 1 |
| PLK2 | 0.274743272 | 2.22E−13 |
| NEXN | 0.27437062 | 8.54E−11 |
| SEC24D | 0.27395841 | 8.01E−12 |
| C11orf24 | 0.27329155 | 5.01E−10 |
| C10orf90 | 0.273012658 | 7.82E−14 |
| NRBF2 | 0.272928691 | 4.97E−11 |
| YRDC | 0.272465098 | 6.79E−11 |
| ADAM12 | 0.272089054 | 3.81E−11 |
| DSE | 0.271615184 | 9.19E−10 |
| GLO1 | 0.269818214 | 1.01E−13 |
| WNT9A | 0.269258272 | 5.10E−17 |
| TMEM218 | 0.268923991 | 1.14E−08 |
| BNC1 | 0.268795705 | 1.05E−22 |
| GPNMB | 0.267056623 | 0.01187383 |
| ENSCAFG00000012041 | 0.266965762 | 1.07E−10 |
| PPP1R15B | 0.266453221 | 1.78E−10 |
| SEMA3C | 0.265622693 | 5.19E−08 |
| STXBP6 | 0.265520629 | 4.60E−08 |
| PTPN12 | 0.265435261 | 4.09E−11 |
| SRSF11 | 0.265125218 | 4.34E−13 |
| MAPK8 | 0.265047739 | 8.76E−12 |
| UGCG | 0.264965223 | 4.27E−06 |
| MSN | 0.264350131 | 5.07E−14 |
| ACTG2 | 0.264339894 | 1 |
| FADS1 | 0.263944354 | 3.63E−06 |
| SLC25A16 | 0.263112844 | 3.93E−11 |
| TIMM8A | 0.262918333 | 7.11E−10 |
| ENSCAFG00000016011 | 0.262799466 | 1.59E−11 |
| SLC20A1 | 0.261386402 | 8.28E−09 |
| PHAX | 0.259855919 | 4.67E−12 |
| FAM46A | 0.259808127 | 0.00076126 |
| C1orf52 | 0.258140183 | 9.52E−09 |
| SNX16 | 0.257966718 | 5.26E−09 |
| STX7 | 0.257533159 | 6.61E−10 |
| EIF1AD | 0.257048668 | 6.20E−12 |
| ENSCAFG00000002833 | 0.256902211 | 1.84E−10 |
| LGALS3 | 0.25589799 | 2.41E−12 |
| ENSCAFG00000009110 | 0.255855497 | 0.00319632 |
| PA2G4 | 0.255149397 | 9.63E−09 |
| DNAJC25 | 0.255086843 | 2.37E−11 |
| MYBL1 | 0.255034351 | 8.97E−07 |
| TGIF2-C20orf24 | 0.254704432 | 4.74E−24 |
| MZT1 | 0.253444595 | 2.30E−19 |
| EGR1 | 0.253387664 | 0.00017755 |
| ABRACL | 0.253085413 | 2.76E−09 |
| DUS4L | 0.253085413 | 1.08E−12 |
| WDR70 | 0.253085413 | 1.20E−11 |
| ENSCAFG00000006693 | 0.252862024 | 1.44E−12 |
| ENSCAFG00000015396 | 0.252221482 | 9.63E−11 |
| MYO1E | 0.251784176 | 2.76E−08 |
| ALG5 | 0.251159176 | 6.87E−13 |
| SYTL4 | 0.250850775 | 1.78E−08 |
| HAUS3 | 0.250669369 | 7.79E−12 |
| LIN7B | 0.250504766 | 9.14E−07 |
| ENSCAFG00000019620 | 0.250464179 | 4.76E−07 |
| ND4L | −0.2502282 | 1.22E−11 |
| ENSCAFG00000006810 | −0.2504346 | 1.18E−35 |
| ENSCAFG00000001968 | −0.25169181 | 3.34E−09 |
| ENSCAFG00000029055 | −0.25234755 | 1.34E−11 |
| KIAA1217 | −0.2529506 | 0.00179526 |
| ENSCAFG00000023205 | −0.25319681 | 1.13E−09 |
| SLC38A2 | −0.25463943 | 3.95E−09 |
| RPL27A | −0.25527852 | 6.77E−19 |
| ATP5F1 | −0.25558736 | 2.46E−12 |
| SYNE2 | −0.25563906 | 0.00564005 |
| FTH1.1 | −0.25601459 | 1.65E−28 |
| IRX2 | −0.25697068 | 4.15E−07 |
| RPS23 | −0.25707733 | 3.24E−66 |
| PLEKHA2 | −0.25732945 | 4.85E−10 |
| TMSB10 | −0.25738773 | 1.06E−07 |
| EFEMP2 | −0.25774021 | 2.43E−06 |
| RPL32 | −0.2580165 | 1.35E−68 |
| RPS16 | −0.26051969 | 6.36E−53 |
| OLFM1 | −0.26165411 | 5.40E−06 |
| MATN2 | −0.26173223 | 0.22784127 |
| CA9 | −0.26259303 | 0.00077323 |
| ENSCAFG00000031486 | −0.26282112 | 3.03E−12 |
| ENSCAFG00000031458 | −0.26366542 | 6.53E−27 |
| EIF3G | −0.2640728 | 1.09E−10 |
| RPS8 | −0.26452057 | 5.76E−52 |
| ENSCAFG00000013201 | −0.26458174 | 1.98E−09 |
| NCBP2 | −0.26478778 | 1.63E−14 |
| IRF3 | −0.26479261 | 2.42E−05 |
| COL11A1 | −0.26495472 | 2.11E−07 |
| ATP5J | −0.26513943 | 7.57E−36 |
| ENSCAFG00000001214 | −0.26698805 | 4.12E−43 |
| HAGH | −0.26721896 | 4.44E−05 |
| SNRPG | −0.26737977 | 1.23E−28 |
| RPS27A | −0.267553 | 7.06E−50 |
| PLA2G7 | −0.26798435 | 8.00E−05 |
| COX7A2 | −0.26828031 | 2.01E−33 |
| CRYL1 | −0.26869155 | 0.03987256 |
| UQCC2 | −0.26872058 | 3.03E−20 |
| RPS19 | −0.26965853 | 2.54E−54 |
| LSM8 | −0.27001268 | 1.71E−17 |
| ENSCAFG00000033694 | −0.27033944 | 1.27E−05 |
| NAA38 | −0.27091997 | 1.03E−13 |
| ENSCAFG00000013214 | −0.27116323 | 0.0001272 |
| ENSCAFG00000015445 | −0.27222156 | 3.77E−14 |
| EIF3H | −0.27225303 | 5.17E−15 |
| FAM162A | −0.27255667 | 5.10E−19 |
| ENSCAFG00000031671 | −0.27326291 | 1.45E−12 |
| DHRS7 | −0.27358298 | 1.57E−13 |
| ENSCAFG00000009523 | −0.27388325 | 3.12E−65 |
| ENSCAFG00000024837 | −0.27503938 | 1.26E−06 |
| CAV1 | −0.27575872 | 1.13E−08 |
| PIK3R1 | −0.27616776 | 6.20E−08 |
| PNP | −0.27624136 | 1.22E−10 |
| RPS3 | −0.27646423 | 4.97E−41 |
| PRDX6 | −0.27721779 | 2.74E−10 |
| SYNE1 | −0.27766239 | 1.12E−06 |
| TGFB2 | −0.27860449 | 1.20E−05 |
| C19orf70 | −0.27913707 | 1.68E−16 |
| RPL9 | −0.27933313 | 3.27E−56 |
| UBA52 | −0.27967552 | 1.06E−61 |
| IRX3 | −0.27994437 | 2.12E−12 |
| ENSCAFG00000000567 | −0.28048729 | 3.78E−32 |
| SIX1 | −0.28063403 | 1.81E−08 |
| ENSCAFG00000031583 | −0.2822734 | 5.57E−05 |
| COL8A2 | −0.28259098 | 2.47E−06 |
| TMEM47 | −0.28263776 | 7.01E−06 |
| RPS14 | −0.28359277 | 3.94E−64 |
| ENSCAFG00000024413 | −0.28407068 | 1.57E−22 |
| EIF4A2 | −0.28532648 | 2.77E−12 |
| RPS15 | −0.28589156 | 1.15E−73 |
| PTRHD1 | −0.28636413 | 2.85E−09 |
| SCARB1 | −0.28673244 | 0.00013137 |
| RPL5 | −0.28692681 | 1.38E−38 |
| MRPL36 | −0.28821789 | 2.66E−19 |
| UQCRQ | −0.28822847 | 3.47E−22 |
| TMEM106C | −0.28847789 | 9.60E−10 |
| NUDT2 | −0.28939319 | 1.74E−10 |
| ENSCAFG00000000061 | −0.28999226 | 3.63E−16 |
| HP1BP3 | −0.29072458 | 3.45E−10 |
| COX4I1 | −0.29091796 | 1.88E−26 |
| MINOS1 | −0.29188549 | 7.83E−13 |
| RPS6 | −0.29213686 | 4.42E−59 |
| ENSCAFG00000007990 | −0.29262724 | 4.16E−53 |
| ENSCAFG00000014234 | −0.29369794 | 3.16E−14 |
| DDOST | −0.29460599 | 4.75E−12 |
| NDUFS5 | −0.29480992 | 3.04E−14 |
| ENSCAFG00000025332 | −0.29552836 | 3.73E−08 |
| OCIAD2 | −0.29661566 | 6.10E−05 |
| RPL23 | −0.29704381 | 4.23E−14 |
| PLCD1 | −0.29814697 | 9.30E−09 |
| ENSCAFG00000008935 | −0.29847055 | 3.16E−16 |
| RPS5 | −0.29888365 | 8.74E−49 |
| KRTCAP2 | −0.29965666 | 3.53E−32 |
| RPS7 | −0.3001245 | 3.11E−61 |
| ADGRG1 | −0.30027415 | 8.02E−07 |
| AK1 | −0.3003055 | 1.74E−17 |
| ETV5 | −0.30032907 | 0.00065529 |
| CUTA | −0.30090167 | 2.16E−15 |
| MACF1 | −0.3025817 | 2.22E−14 |
| S100A13 | −0.30389326 | 3.77E−21 |
| RPL11 | −0.3055851 | 1.77E−61 |
| KIAA1551 | −0.30634507 | 7.15E−05 |
| DDX41 | −0.30678774 | 1.74E−09 |
| ENSCAFG00000010850 | −0.30709786 | 7.99E−21 |
| ENSCAFG00000004800 | −0.30879851 | 8.64E−20 |
| RPL14 | −0.30950893 | 3.75E−53 |
| ATPIF1 | −0.31009434 | 1.05E−30 |
| ENSCAFG00000024445 | −0.31072658 | 8.93E−17 |
| ENSCAFG00000019141 | −0.31082896 | 3.03E−12 |
| PDGFRA | −0.31213428 | 3.66E−09 |
| MYLIP | −0.31239862 | 0.00305231 |
| WNK1 | −0.31371948 | 2.85E−06 |
| RPL15 | −0.31381374 | 6.96E−58 |
| CPNE2 | −0.31450523 | 8.40E−11 |
| NSMCE1 | −0.31690314 | 4.08E−15 |
| ENSCAFG00000008336 | −0.31773946 | 7.54E−15 |
| S100A6 | −0.31834723 | 7.14E−29 |
| ENSCAFG00000029635 | −0.31853919 | 2.16E−27 |
| RPL23.1 | −0.31897707 | 4.47E−75 |
| ALDOC | −0.3202288 | 1.44E−05 |
| MET | −0.32041132 | 1.58E−12 |
| RPL28 | −0.32114441 | 1.45E−72 |
| MT-ND3 | −0.32117437 | 5.90E−24 |
| sep [symbol] | −0.32177457 | 5.49E−06 |
| ENSCAFG00000012725 | −0.3241188 | 9.34E−84 |
| ENSCAFG00000008776 | −0.32856626 | 4.48E−24 |
| CAV2 | −0.32918041 | 6.60E−21 |
| ENSCAFG00000012395 | −0.3294887 | 3.14E−79 |
| RACK1 | −0.33114297 | 7.96E−31 |
| FAIM | −0.33213103 | 8.41E−10 |
| PERP | −0.33359759 | 1.78E−15 |
| ENSCAFG00000012553 | −0.33360054 | 5.06E−07 |
| ENSCAFG00000006545 | −0.33425112 | 8.50E−24 |
| CDON | −0.3359286 | 5.67E−05 |
| DCXR | −0.33648006 | 3.65E−14 |
| ENSCAFG00000012676 | −0.33764386 | 8.57E−50 |
| RPS9 | −0.33797823 | 1.18E−68 |
| NR4A2 | −0.33836487 | 1 |
| EGLN1 | −0.33838955 | 1.26E−14 |
| RPL7A | −0.33906574 | 7.16E−43 |
| SERTAD4 | −0.34066495 | 4.26E−08 |
| RPL31 | −0.34097289 | 9.02E−43 |
| PRDX4 | −0.34132246 | 1.23E−05 |
| LSP1 | −0.34237651 | 4.73E−06 |
| PFDN5.1 | −0.34415804 | 3.86E−40 |
| THRAP3 | −0.34500485 | 2.12E−15 |
| SPTLC3 | −0.34527319 | 1.07E−06 |
| RPL27 | −0.34716721 | 6.72E−83 |
| COX7A1 | −0.3477697 | 1.62E−12 |
| ENSCAFG00000031952 | −0.34871773 | 1.69E−75 |
| ENSCAFG00000006734 | −0.34916722 | 4.98E−47 |
| CD59 | −0.34939808 | 2.99E−08 |
| ENSCAFG00000014648 | −0.35067979 | 1.17E−31 |
| TMA7 | −0.35455243 | 6.98E−28 |
| ENSCAFG00000000377 | −0.35513972 | 3.26E−65 |
| ENSCAFG00000008873 | −0.35553678 | 3.16E−29 |
| SSBP2 | −0.35603916 | 1.06E−16 |
| TSTD1 | −0.35753604 | 3.81E−20 |
| NAP1L1 | −0.35787215 | 2.53E−31 |
| ENSCAFG00000007199 | −0.35808819 | 1.69E−07 |
| IFI30 | −0.35851738 | 1.95E−16 |
| RPS18 | −0.35930671 | 1.63E−66 |
| ITM2C | −0.35992537 | 1.12E−11 |
| GLI3 | −0.36064372 | 9.71E−16 |
| RPS4X | −0.36073595 | 7.39E−68 |
| RPS17 | −0.36250787 | 2.54E−86 |
| MALL | −0.36321906 | 0.00544896 |
| NDUFB2 | −0.363249 | 1.47E−47 |
| ENSCAFG00000012839 | −0.36479984 | 2.36E−28 |
| ANLN | −0.36493066 | 4.03E−08 |
| NPNT | −0.3652741 | 0.00040752 |
| ENSCAFG00000033303 | −0.36765835 | 1.85E−11 |
| PTMA | −0.37007399 | 9.31E−13 |
| SSR4 | −0.37115863 | 1.71E−49 |
| ENSCAFG00000018187 | −0.37257065 | 9.37E−30 |
| CDKN2C | −0.37273569 | 3.27E−14 |
| ARID5B | −0.37401241 | 3.20E−10 |
| ENSCAFG00000032537 | −0.37489109 | 2.01E−29 |
| FTL | −0.37512085 | 6.70E−37 |
| RPL10A | −0.37521784 | 5.96E−69 |
| timp3 | −0.3754959 | 1.45E−06 |
| EFEMP1 | −0.37717726 | 1.66E−05 |
| BNIP3 | −0.37754141 | 3.43E−18 |
| NFIB | −0.37790027 | 3.52E−16 |
| ENSCAFG00000004260 | −0.38047277 | 4.07E−42 |
| ENSCAFG00000030466 | −0.38162162 | 1.15E−20 |
| KLF5 | −0.38266125 | 5.01E−10 |
| ENSCAFG00000015433 | −0.38290335 | 3.48E−35 |
| TRPS1 | −0.38607454 | 9.04E−23 |
| GLTSCR2 | −0.38646882 | 8.82E−17 |
| LRIG1 | −0.38741804 | 0.45650294 |
| RPL35 | −0.38837899 | 6.40E−96 |
| EPS8 | −0.38939014 | 1.05E−15 |
| ENSCAFG00000018784 | −0.39083723 | 3.47E−79 |
| RPS15A | −0.39507679 | 1.47E−95 |
| ENSCAFG00000016065 | −0.39527975 | 4.76E−29 |
| EIF3E | −0.39793997 | 4.14E−30 |
| SOX9 | −0.39832216 | 2.28E−15 |
| HSP90AB1 | −0.39919468 | 1.14E−23 |
| RPL7.1 | −0.40252969 | 9.64E−57 |
| USP6NL | −0.4044903 | 9.81E−09 |
| KARS | −0.40685681 | 4.09E−29 |
| ENSCAFG00000032152 | −0.40705689 | 2.87E−50 |
| FOS | −0.40760097 | 6.05E−12 |
| EIF3K | −0.40791741 | 1.51E−55 |
| ENSCAFG00000031706 | −0.40811952 | 5.87E−14 |
| HMGB2 | −0.41088923 | 3.15E−13 |
| CCND1 | −0.41110376 | 3.79E−12 |
| GM2A | −0.41336979 | 2.29E−12 |
| AIF1L | −0.41497493 | 1.01E−12 |
| ENSCAFG00000028936 | −0.41544652 | 1.22E−15 |
| RPS27 | −0.41766771 | 2.25E−84 |
| RDH10 | −0.41857351 | 1 |
| DBI | −0.42212407 | 1.39E−26 |
| EEF1D | −0.42420928 | 1.23E−48 |
| ENSCAFG00000031955 | −0.42524668 | 3.47E−101 |
| LIMCH1 | −0.42552716 | 1.02E−12 |
| ENSCAFG00000014958 | −0.42561197 | 1.06E−84 |
| PPP1R1B | −0.43481929 | 9.79E−16 |
| ENSCAFG00000029945 | −0.4358663 | 1.27E−51 |
| ABLIM1 | −0.43808576 | 4.33E−09 |
| MEI4 | −0.43823361 | 6.55E−65 |
| CST6 | −0.43891285 | 6.44E−21 |
| ENSCAFG00000028960 | −0.4393504 | 2.84E−44 |
| ENSCAFG00000012624 | −0.43958546 | 1.07E−36 |
| PPP1R3C | −0.4414792 | 2.49E−13 |
| ZFHX3 | −0.44526818 | 4.05E−21 |
| FRMD6 | −0.44801131 | 5.66E−19 |
| RPL3 | −0.44877661 | 3.98E−61 |
| ENSCAFG00000003564 | −0.4536463 | 1.24E−44 |
| DEK | −0.45464618 | 3.20E−21 |
| ENSCAFG00000032920 | −0.45475746 | 2.07E−27 |
| ZBTB16 | −0.45556596 | 6.23E−25 |
| ENSCAFG00000019044 | −0.46149169 | 4.79E−97 |
| INSIG1 | −0.46389424 | 2.41E−15 |
| FBXO2 | −0.46751861 | 2.22E−13 |
| IL18BP | −0.4702197 | 0.00212428 |
| EIF3L | −0.47603295 | 4.38E−32 |
| TFRC | −0.48601212 | 2.49E−21 |
| TSPAN13 | −0.48711431 | 7.58E−11 |
| PTGDR | −0.49035848 | 0.00147465 |
| BGLAP | −0.4908875 | 0.00032211 |
| GOLIM4 | −0.49288747 | 9.91E−13 |
| NUDT4 | −0.49350073 | 4.93E−15 |
| ENSCAFG00000032717 | −0.49606124 | 4.74E−93 |
| ZNF395 | −0.50102873 | 5.16E−29 |
| ENSCAFG00000024643 | −0.50180857 | 1.14E−49 |
| TSC22D3 | −0.50190377 | 2.67E−22 |
| CRYAB | −0.5056394 | 1.42E−14 |
| BBX | −0.50647686 | 6.73E−27 |
| ENSCAFG00000015834 | −0.51931737 | 3.02E−77 |
| ENSCAFG00000015372 | −0.51998521 | 3.15E−22 |
| BMP4 | −0.52039254 | 1 |
| SLC1A1 | −0.52843445 | 1.88E−10 |
| CADM1 | −0.5363386 | 3.68E−16 |
| ENSCAFG00000000027 | −0.5368636 | 3.09E−68 |
| AHNAK | −0.53702784 | 1.49E−15 |
| ENSCAFG00000017688 | −0.53835656 | 1.13E−100 |
| IMPDH2 | −0.5387333 | 2.55E−32 |
| FDFT1 | −0.55181825 | 3.95E−23 |
| TFAP2C | −0.55331187 | 8.72E−22 |
| ENSCAFG00000006884 | −0.55630239 | 6.04E−58 |
| APOE | −0.55635733 | 0.00074245 |
| MEF2C | −0.55718713 | 1.64E−21 |
| GSTM3 | −0.5694882 | 5.24E−14 |
| OGFRL1 | −0.57110992 | 4.48E−15 |
| TM4SF18 | −0.59863723 | 5.61E−19 |
| PRELP | −0.61360364 | 1.32E−22 |
| TNNI2 | −0.61963121 | 2.31E−18 |
| TGFBR3 | −0.62222398 | 4.53E−21 |
| IL33 | −0.627616 | 1.88E−13 |
| ABI3BP | −0.63172183 | 1.59E−14 |
| CLU | −0.63292644 | 1.21E−13 |
| SCG5 | −0.63496211 | 9.07E−22 |
| NDP | −0.64329834 | 4.58E−20 |
| ENSCAFG00000030307 | −0.64707163 | 7.11E−17 |
| ENSCAFG00000029553 | −0.66078747 | 1 |
| CTSD | −0.66916005 | 8.47E−14 |
| EIF4B | −0.68413781 | 7.28E−63 |
| EEF1A1 | −0.69294622 | 3.17E−104 |
| EDIL3 | −0.69733609 | 1.75E−28 |
| MGST1 | −0.72985887 | 3.99E−21 |
| B2M | −0.74207749 | 8.44E−26 |
| SHISA2 | −0.74376072 | 1.29E−24 |
| FGF7 | −0.74634343 | 3.26E−20 |
| FAM213B | −0.7710177 | 1.28E−22 |
| ENSCAFG00000018746 | −0.79340807 | 3.79E−22 |
| TCF7L2 | −0.85381978 | 1.01E−74 |
| ACAN | −0.87659982 | 9.99E−09 |
| ENSCAFG00000028765 | −0.93655397 | 3.09E−38 |
| MAL | −0.95458723 | 2.85E−22 |
| HAPLN1 | −0.98651343 | 1.02E−21 |
| FXYD6 | −0.98930748 | 2.20E−27 |
| FAM180A | −0.99904243 | 2.67E−19 |
| IFI6 | −1.00174878 | 6.45E−06 |
| ENSCAFG00000012963 | −1.04699494 | 1.30E−28 |
| CDO1 | −1.25074668 | 3.02E−17 |
| TF.1 | −1.25650717 | 1.71E−24 |
| SCRG1 | −1.30522628 | 1.67E−29 |
| C2orf40 | −1.34826821 | 2.43E−35 |
| COL2A1 | −1.59138813 | 8.90E−44 |

[symbol]

indicates data missing or illegible when filed

TABLE 2B

| Genes | avg_logFC | p_val_adj |
|---|---|---|
| SERPINE1 | 2.270134566 | 1.64E−124 |
| PLAU | 1.938321329 | 3.33E−101 |
| TF | 1.615323222 | 8.66E−70 |
| CYP1B1 | 1.605188409 | 3.59E−52 |
| PTGS2 | 1.601522321 | 2.60E−53 |
| CTGF | 1.594792721 | 2.67E−101 |
| GADD45B | 1.575014248 | 2.41E−111 |
| PMEPA1 | 1.368238395 | 1.77E−138 |
| RGS2 | 1.33586132 | 3.82E−30 |
| LIF | 1.185820503 | 2.35E−68 |
| ENSCAFG00000007045 | 1.127510697 | 4.59E−56 |
| PLAUR | 1.080355294 | 2.92E−83 |
| BHLHE40 | 0.987427044 | 2.44E−75 |
| STC2 | 0.973849543 | 1.62E−60 |
| GDNF | 0.951146608 | 1.94E−65 |
| INHBA | 0.938581446 | 1.09E−42 |
| FSTL3 | 0.915068598 | 1.25E−68 |
| ALAS1 | 0.910462856 | 9.86E−46 |
| DDX58 | 0.907727781 | 7.51E−05 |
| SNAI1 | 0.880863852 | 3.42E−59 |
| HAS2 | 0.831263218 | 1.42E−38 |
| CYR61 | 0.822041938 | 1.81E−55 |
| VCAN | 0.791744247 | 3.50E−53 |
| HBEGF | 0.743209509 | 3.29E−31 |
| LTBP1 | 0.726556801 | 6.54E−43 |
| UGDH | 0.723736501 | 2.68E−54 |
| SKIL | 0.722523271 | 1.13E−68 |
| ENSCAFG00000032813 | 0.69050212 | 2.99E−22 |
| CYP26B1 | 0.66026285 | 7.51E−06 |
| EDN1 | 0.659509753 | 1.23E−11 |
| FST | 0.649885638 | 1.53E−06 |
| RASL11A | 0.643700697 | 1 |
| PTHLH | 0.632177886 | 5.00E−13 |
| TFPI2 | 0.631465532 | 3.09E−31 |
| BAZ1A | 0.625318472 | 3.17E−59 |
| LRRC8C | 0.613489029 | 3.03E−43 |
| PDPN | 0.612788854 | 1.17E−25 |
| ISG20 | 0.610179996 | 1.71E−28 |
| ACTN1 | 0.607997286 | 7.22E−54 |
| THBS1 | 0.572688209 | 1.13E−34 |
| ENSCAFG00000015625 | 0.569353099 | 4.40E−22 |
| ENSCAFG00000002015 | 0.566931371 | 2.05E−46 |
| CYCS | 0.565808469 | 3.55E−63 |
| PDE4B | 0.564477965 | 1.51E−27 |
| COL5A1 | 0.55491057 | 6.15E−27 |
| PDLIM5 | 0.55338401 | 5.05E−42 |
| ELOVL4 | 0.541004424 | 8.53E−32 |
| ICAM1 | 0.540189379 | 1.40E−31 |
| FOXP4 | 0.538976974 | 5.14E−38 |
| NGF | 0.536034633 | 7.86E−25 |
| RBMS1 | 0.520889735 | 1.39E−56 |
| ACTA2 | 0.51835637 | 4.77E−05 |
| ENSCAFG00000033998 | 0.510076931 | 2.01E−21 |
| ADSS | 0.508717264 | 2.27E−29 |
| WISP1 | 0.505862364 | 1.47E−14 |
| ELK3 | 0.505306706 | 4.15E−28 |
| MAP1B | 0.501889012 | 3.96E−29 |
| S100A2 | 0.501136569 | 1 |
| MARCH3 | 0.495772749 | 1.36E−25 |
| NF2 | 0.494490521 | 4.59E−16 |
| ENSCAFG00000003758 | 0.49399392 | 1.14E−30 |
| SNRNP48 | 0.489452297 | 3.82E−37 |
| NIPAL1 | 0.488938551 | 9.16E−25 |
| TNFSF10 | 0.487114772 | 1.26E−27 |
| MT2A | 0.484966088 | 9.09E−27 |
| MEDAG | 0.483035018 | 5.29E−14 |
| COL1A1 | 0.481300099 | 2.93E−14 |
| IFRD1 | 0.47940483 | 1.85E−26 |
| DNAJB4 | 0.478466459 | 2.86E−25 |
| KCTD10 | 0.472668513 | 7.71E−34 |
| RCAN1 | 0.467451478 | 2.95E−19 |
| PALLD | 0.46704451 | 3.09E−25 |
| FZD2 | 0.461441264 | 3.70E−42 |
| KIF5B | 0.452934638 | 9.92E−51 |
| FN1 | 0.451706657 | 7.67E−21 |
| ENSCAFG00000006436 | 0.450184308 | 9.50E−28 |
| GLRX | 0.447673859 | 7.04E−08 |
| VPS37A | 0.44658027 | 6.47E−31 |
| SLC4A7 | 0.44656017 | 8.43E−31 |
| ENSCAFG00000037735 | 0.44491503 | 9.27E−20 |
| NEK7 | 0.440671399 | 1.16E−23 |
| ENSCAFG00000024171 | 0.439808333 | 2.72E−13 |
| PLOD2 | 0.435799011 | 5.58E−32 |
| SSX2IP | 0.433433068 | 5.40E−22 |
| HIP1 | 0.430291869 | 2.35E−28 |
| ELL2 | 0.429880706 | 4.21E−26 |
| PTK7 | 0.428408906 | 2.31E−26 |
| ANO6 | 0.42819938 | 6.54E−27 |
| ETS1 | 0.42651363 | 1.16E−23 |
| COL8A1 | 0.425783098 | 1.12E−05 |
| IQCJ-SCHIP1 | 0.421507132 | 2.17E−15 |
| LOX | 0.421443873 | 1.73E−18 |
| SH3BP5L | 0.420108354 | 8.54E−23 |
| RAI14 | 0.418838543 | 5.16E−24 |
| SNX10 | 0.416905955 | 2.20E−24 |
| ADAM19 | 0.416225598 | 7.29E−18 |
| COL12A1 | 0.415635917 | 3.05E−22 |
| HSPH1 | 0.412816956 | 6.08E−27 |
| SRPX2 | 0.412666738 | 1.53E−24 |
| DLC1 | 0.411009682 | 2.22E−19 |
| ALDH18A1 | 0.410604989 | 6.49E−33 |
| AMOTL2 | 0.410370891 | 1.12E−18 |
| SDC4 | 0.406600535 | 1.73E−09 |
| PFKFB3 | 0.403011464 | 1 |
| NRP2 | 0.402252102 | 9.73E−24 |
| ENSCAFG00000000345 | 0.398102466 | 6.59E−26 |
| CRABP2 | 0.396979592 | 2.93E−14 |
| GORAB | 0.396186256 | 1.23E−15 |
| TPM2 | 0.394423601 | 0.0010039 |
| LUM | 0.392747857 | 2.43E−05 |
| ENSCAFG00000013979 | 0.388719855 | 3.97E−14 |
| SPHK1 | 0.38692647 | 4.99E−28 |
| IFIH1 | 0.38656085 | 7.45E−06 |
| COL3A1 | 0.383267542 | 4.72E−13 |
| BGN | 0.383210386 | 5.66E−14 |
| PICALM | 0.382048887 | 1.62E−29 |
| RALA | 0.377466022 | 4.13E−22 |
| ITGA5 | 0.372426688 | 0.00022458 |
| ZNFX1 | 0.371873777 | 2.03E−23 |
| ENSCAFG00000036684 | 0.371392695 | 1.83E−17 |
| MAP7D1 | 0.367099126 | 8.37E−18 |
| SRM | 0.366802221 | 1.71E−19 |
| BCL2L11 | 0.366623544 | 1.54E−14 |
| AEN | 0.366556769 | 4.58E−09 |
| MYH9 | 0.365525825 | 1.82E−23 |
| YIF1B | 0.364924894 | 5.58E−20 |
| NFKBIA | 0.363974328 | 0.00098373 |
| PHF20L1 | 0.363486584 | 5.95E−20 |
| PDZRN3 | 0.361763681 | 3.68E−24 |
| BLOC1S2 | 0.360110531 | 8.67E−25 |
| GTF2F1 | 0.359984498 | 7.39E−18 |
| PTGES | 0.354877191 | 1 |
| SPOP | 0.354769454 | 3.71E−17 |
| BCAR1 | 0.354670991 | 5.43E−18 |
| GFPT2 | 0.353801801 | 3.84E−08 |
| CKAP4 | 0.353447818 | 6.34E−26 |
| ENSCAFG00000031852 | 0.351467487 | 9.04E−27 |
| TUBA4A | 0.351136866 | 3.09E−12 |
| PNPLA2 | 0.351032625 | 5.69E−18 |
| CTNNB1 | 0.348949126 | 7.58E−35 |
| FAS | 0.348560418 | 7.60E−17 |
| SYNC | 0.346548987 | 2.88E−07 |
| MMD | 0.345335146 | 1.13E−15 |
| TCF4 | 0.34342355 | 2.19E−13 |
| TCIRG1 | 0.341695915 | 1.25E−15 |
| IBTK | 0.341267978 | 1.95E−18 |
| GOLT1B | 0.340457605 | 3.76E−21 |
| SNRNP40 | 0.34009679 | 1.00E−17 |
| COL1A2 | 0.339411575 | 2.05E−10 |
| HAPLN3 | 0.33610249 | 5.47E−13 |
| SACS | 0.33507603 | 4.06E−13 |
| PINX1 | 0.334967829 | 2.96E−13 |
| CDK8 | 0.334822383 | 1.34E−18 |
| ENSCAFG00000011482 | 0.332844714 | 2.69E−14 |
| UBE2J1 | 0.332459 | 8.26E−16 |
| S100A4 | 0.331511753 | 4.16E−05 |
| STARD13 | 0.330863114 | 7.13E−12 |
| SRSF7 | 0.330639938 | 2.33E−16 |
| MPP6 | 0.330320089 | 1.35E−13 |
| STK38L | 0.330194066 | 1.22E−11 |
| VEGFA | 0.326552564 | 7.11E−07 |
| GNL3 | 0.326489092 | 1.78E−15 |
| COL5A2 | 0.325283785 | 1.90E−16 |
| PID1 | 0.324789537 | 5.20E−07 |
| NUAK1 | 0.322608179 | 7.38E−12 |
| LMCD1 | 0.322505239 | 0.00031673 |
| LPCAT2 | 0.321883143 | 2.06E−12 |
| THBS2 | 0.321515274 | 9.22E−06 |
| ENSCAFG00000037410 | 0.321415081 | 1.18E−16 |
| ATP13A3 | 0.320744061 | 8.61E−15 |
| ANKRD10 | 0.320687221 | 2.04E−14 |
| TMEM2 | 0.320271599 | 6.72E−11 |
| ENSCAFG00000018440 | 0.318205368 | 3.56E−12 |
| REL | 0.316388368 | 3.30E−17 |
| SNAI2 | 0.316301052 | 1 |
| ENSCAFG00000001840 | 0.315952691 | 6.09E−13 |
| TXNRD1 | 0.314990868 | 2.15E−18 |
| RIC1 | 0.31260878 | 2.06E−14 |
| SMC5 | 0.312166654 | 8.07E−13 |
| NDUFAF4 | 0.312125069 | 1.97E−13 |
| NOP58 | 0.312027948 | 1.77E−09 |
| CSRP1 | 0.311483153 | 1.84E−23 |
| TMEM41B | 0.311216589 | 2.61E−12 |
| ENSCAFG00000017326 | 0.309316441 | 0.65348525 |
| NUDC | 0.308853972 | 1.66E−19 |
| STAM2 | 0.308195198 | 4.20E−20 |
| ENSCAFG00000001448 | 0.307218421 | 4.61E−30 |
| MYLK | 0.307130976 | 2.61E−09 |
| ACVR1 | 0.30491048 | 5.65E−13 |
| RNF6 | 0.304133178 | 1.63E−16 |
| CCT4 | 0.303922731 | 2.48E−17 |
| FAM49B | 0.302795732 | 1.45E−13 |
| TKT | 0.302581621 | 8.90E−06 |
| PCSK1 | 0.302052584 | 1.88E−31 |
| SMG7 | 0.301762461 | 1.82E−12 |
| MRPS31 | 0.301222195 | 6.87E−19 |
| TUBA1C | 0.300991012 | 2.83E−10 |
| UBE2S | 0.300842341 | 1.06E−13 |
| NUDCD1 | 0.29796364 | 2.49E−14 |
| CRLF3 | 0.297570697 | 9.93E−15 |
| TWF1 | 0.296148932 | 6.20E−16 |
| DDX60 | 0.295595225 | 1 |
| GTF2H1 | 0.29497148 | 3.45E−15 |
| FAM126A | 0.294401112 | 3.95E−12 |
| PNPLA8 | 0.292016173 | 1.55E−15 |
| FAM20C | 0.291791458 | 2.00E−09 |
| MT1 | 0.290657433 | 0.14826836 |
| DNAJB9 | 0.289623435 | 2.82E−12 |
| ENSCAFG00000029851 | 0.289545577 | 3.15E−14 |
| PRPF38B | 0.288814072 | 1.03E−13 |
| RPS6KC1 | 0.288104439 | 1.19E−14 |
| DKK1 | 0.288032443 | 3.79E−07 |
| ENSCAFG00000030449 | 0.285576385 | 5.59E−07 |
| DPP4 | 0.285457297 | 2.29E−11 |
| FLNB | 0.284752033 | 1.74E−16 |
| PLS3 | 0.284373241 | 5.79E−12 |
| CLCF1 | 0.284081513 | 4.42E−24 |
| ATP6V1H | 0.280867753 | 4.10E−12 |
| GPATCH4 | 0.280788015 | 3.35E−09 |
| ENSCAFG00000007306 | 0.279060899 | 1.93E−13 |
| TMF1 | 0.278899959 | 4.01E−12 |
| MAOA | 0.277829558 | 1.24E−11 |
| NAV3 | 0.277094704 | 1.59E−10 |
| SOD2 | 0.276366887 | 1.30E−12 |
| EMD | 0.276004674 | 1.41E−10 |
| ANKRD37 | 0.275977979 | 1 |
| PLK2 | 0.274743272 | 2.22E−13 |
| NEXN | 0.27437062 | 8.54E−11 |
| SEC24D | 0.27395841 | 8.01E−12 |
| C11orf24 | 0.27329155 | 5.01E−10 |
| C10orf90 | 0.273012658 | 7.82E−14 |
| NRBF2 | 0.272928691 | 4.97E−11 |
| YRDC | 0.272465098 | 6.79E−11 |
| ADAM12 | 0.272089054 | 3.81E−11 |
| DSE | 0.271615184 | 9.19E−10 |
| GLO1 | 0.269818214 | 1.01E−13 |
| WNT9A | 0.269258272 | 5.10E−17 |
| TMEM218 | 0.268923991 | 1.14E−08 |
| BNC1 | 0.268795705 | 1.05E−22 |
| GPNMB | 0.267056623 | 0.01187383 |
| ENSCAFG00000012041 | 0.266965762 | 1.07E−10 |
| PPP1R15B | 0.266453221 | 1.78E−10 |
| SEMA3C | 0.265622693 | 5.19E−08 |
| STXBP6 | 0.265520629 | 4.60E−08 |
| PTPN12 | 0.265435261 | 4.09E−11 |
| SRSF11 | 0.265125218 | 4.34E−13 |
| MAPK8 | 0.265047739 | 8.76E−12 |
| UGCG | 0.264965223 | 4.27E−06 |
| MSN | 0.264350131 | 5.07E−14 |
| ACTG2 | 0.264339894 | 1 |
| FADS1 | 0.263944354 | 3.63E−06 |
| SLC25A16 | 0.263112844 | 3.93E−11 |
| TIMM8A | 0.262918333 | 7.11E−10 |
| ENSCAFG00000016011 | 0.262799466 | 1.59E−11 |
| SLC20A1 | 0.261386402 | 8.28E−09 |
| PHAX | 0.259855919 | 4.67E−12 |
| FAM46A | 0.259808127 | 0.00076126 |
| C1orf52 | 0.258140183 | 9.52E−09 |
| SNX16 | 0.257966718 | 5.26E−09 |
| STX7 | 0.257533159 | 6.61E−10 |
| EIF1AD | 0.257048668 | 6.20E−12 |
| ENSCAFG00000002833 | 0.256902211 | 1.84E−10 |
| LGALS3 | 0.25589799 | 2.41E−12 |
| ENSCAFG00000009110 | 0.255855497 | 0.00319632 |
| PA2G4 | 0.255149397 | 9.63E−09 |
| DNAJC25 | 0.255086843 | 2.37E−11 |
| MYBL1 | 0.255034351 | 8.97E−07 |
| TGIF2-C20orf24 | 0.254704432 | 4.74E−24 |
| MZT1 | 0.253444595 | 2.30E−19 |
| EGR1 | 0.253387664 | 0.00017755 |
| ABRACL | 0.253085413 | 2.76E−09 |
| DUS4L | 0.253085413 | 1.08E−12 |
| WDR70 | 0.253085413 | 1.20E−11 |
| ENSCAFG00000006693 | 0.252862024 | 1.44E−12 |
| ENSCAFG00000015396 | 0.252221482 | 9.63E−11 |
| MYO1E | 0.251784176 | 2.76E−08 |
| ALG5 | 0.251159176 | 6.87E−13 |
| SYTL4 | 0.250850775 | 1.78E−08 |
| HAUS3 | 0.250669369 | 7.79E−12 |
| LIN7B | 0.250504766 | 9.14E−07 |
| ENSCAFG00000019620 | 0.250464179 | 4.76E−07 |
| ND4L | −0.2502282 | 1.22E−11 |
| ENSCAFG00000006810 | −0.2504346 | 1.18E−35 |
| ENSCAFG00000001968 | −0.25169181 | 3.34E−09 |
| ENSCAFG00000029055 | −0.25234755 | 1.34E−11 |
| KIAA1217 | −0.2529506 | 0.00179526 |
| ENSCAFG00000023205 | −0.25319681 | 1.13E−09 |
| SLC38A2 | −0.25463943 | 3.95E−09 |
| RPL27A | −0.25527852 | 6.77E−19 |
| ATP5F1 | −0.25558736 | 2.46E−12 |
| SYNE2 | −0.25563906 | 0.00564005 |
| FTH1.1 | −0.25601459 | 1.65E−28 |
| IRX2 | −0.25697068 | 4.15E−07 |
| RPS23 | −0.25707733 | 3.24E−66 |
| PLEKHA2 | −0.25732945 | 4.85E−10 |
| TMSB10 | −0.25738773 | 1.06E−07 |
| EFEMP2 | −0.25774021 | 2.43E−06 |
| RPL32 | −0.2580165 | 1.35E−68 |
| RPS16 | −0.26051969 | 6.36E−53 |
| OLFM1 | −0.26165411 | 5.40E−06 |
| MATN2 | −0.26173223 | 0.22784127 |
| CA9 | −0.26259303 | 0.00077323 |
| ENSCAFG00000031486 | −0.26282112 | 3.03E−12 |
| ENSCAFG00000031458 | −0.26366542 | 6.53E−27 |
| EIF3G | −0.2640728 | 1.09E−10 |
| RPS8 | −0.26452057 | 5.76E−52 |
| ENSCAFG00000013201 | −0.26458174 | 1.98E−09 |
| NCBP2 | −0.26478778 | 1.63E−14 |
| IRF3 | −0.26479261 | 2.42E−05 |
| COL11A1 | −0.26495472 | 2.11E−07 |
| ATP5J | −0.26513943 | 7.57E−36 |
| ENSCAFG00000001214 | −0.26698805 | 4.12E−43 |
| HAGH | −0.26721896 | 4.44E−05 |
| SNRPG | −0.26737977 | 1.23E−28 |
| RPS27A | −0.267553 | 7.06E−50 |
| PLA2G7 | −0.26798435 | 8.00E−05 |
| COX7A2 | −0.26828031 | 2.01E−33 |
| CRYL1 | −0.26869155 | 0.03987256 |
| UQCC2 | −0.26872058 | 3.03E−20 |
| RPS19 | −0.26965853 | 2.54E−54 |
| LSM8 | −0.27001268 | 1.71E−17 |
| ENSCAFG00000033694 | −0.27033944 | 1.27E−05 |
| NAA38 | −0.27091997 | 1.03E−13 |
| ENSCAFG00000013214 | −0.27116323 | 0.0001272 |
| ENSCAFG00000015445 | −0.27222156 | 3.77E−14 |
| EIF3H | −0.27225303 | 5.17E−15 |
| FAM162A | −0.27255667 | 5.10E−19 |
| ENSCAFG00000031671 | −0.27326291 | 1.45E−12 |
| DHRS7 | −0.27358298 | 1.57E−13 |
| ENSCAFG00000009523 | −0.27388325 | 3.12E−65 |
| ENSCAFG00000024837 | −0.27503938 | 1.26E−06 |
| CAV1 | −0.27575872 | 1.13E−08 |
| PIK3R1 | −0.27616776 | 6.20E−08 |
| PNP | −0.27624136 | 1.22E−10 |
| RPS3 | −0.27646423 | 4.97E−41 |
| PRDX6 | −0.27721779 | 2.74E−10 |
| SYNE1 | −0.27766239 | 1.12E−06 |
| TGFB2 | −0.27860449 | 1.20E−05 |
| C19orf70 | −0.27913707 | 1.68E−16 |
| RPL9 | −0.27933313 | 3.27E−56 |
| UBA52 | −0.27967552 | 1.06E−61 |
| IRX3 | −0.27994437 | 2.12E−12 |
| ENSCAFG00000000567 | −0.28048729 | 3.78E−32 |
| SIX1 | −0.28063403 | 1.81E−08 |
| ENSCAFG00000031583 | −0.2822734 | 5.57E−05 |
| COL8A2 | −0.28259098 | 2.47E−06 |
| TMEM47 | −0.28263776 | 7.01E−06 |
| RPS14 | −0.28359277 | 3.94E−64 |
| ENSCAFG00000024413 | −0.28407068 | 1.57E−22 |
| EIF4A2 | −0.28532648 | 2.77E−12 |
| RPS15 | −0.28589156 | 1.15E−73 |
| PTRHD1 | −0.28636413 | 2.85E−09 |
| SCARB1 | −0.28673244 | 0.00013137 |
| RPL5 | −0.28692681 | 1.38E−38 |
| MRPL36 | −0.28821789 | 2.66E−19 |
| UQCRQ | −0.28822847 | 3.47E−22 |
| TMEM106C | −0.28847789 | 9.60E−10 |
| NUDT2 | −0.28939319 | 1.74E−10 |
| ENSCAFG00000000061 | −0.28999226 | 3.63E−16 |
| HP1BP3 | −0.29072458 | 3.45E−10 |
| COX4I1 | −0.29091796 | 1.88E−26 |
| MINOS1 | −0.29188549 | 7.83E−13 |
| RPS6 | −0.29213686 | 4.42E−59 |
| ENSCAFG00000007990 | −0.29262724 | 4.16E−53 |
| ENSCAFG00000014234 | −0.29369794 | 3.16E−14 |
| DDOST | −0.29460599 | 4.75E−12 |
| NDUFS5 | −0.29480992 | 3.04E−14 |
| ENSCAFG00000025332 | −0.29552836 | 3.73E−08 |
| OCIAD2 | −0.29661566 | 6.10E−05 |
| RPL23 | −0.29704381 | 4.23E−14 |
| PLCD1 | −0.29814697 | 9.30E−09 |
| ENSCAFG00000008935 | −0.29847055 | 3.16E−16 |
| RPS5 | −0.29888365 | 8.74E−49 |
| KRTCAP2 | −0.29965666 | 3.53E−32 |
| RPS7 | −0.3001245 | 3.11E−61 |
| ADGRG1 | −0.30027415 | 8.02E−07 |
| AK1 | −0.3003055 | 1.74E−17 |
| ETV5 | −0.30032907 | 0.00065529 |
| CUTA | −0.30090167 | 2.16E−15 |
| MACF1 | −0.3025817 | 2.22E−14 |
| S100A13 | −0.30389326 | 3.77E−21 |
| RPL11 | −0.3055851 | 1.77E−61 |
| KIAA1551 | −0.30634507 | 7.15E−05 |
| DDX41 | −0.30678774 | 1.74E−09 |
| ENSCAFG00000010850 | −0.30709786 | 7.99E−21 |
| ENSCAFG00000004800 | −0.30879851 | 8.64E−20 |
| RPL14 | −0.30950893 | 3.75E−53 |
| ATPIF1 | −0.31009434 | 1.05E−30 |
| ENSCAFG00000024445 | −0.31072658 | 8.93E−17 |
| ENSCAFG00000019141 | −0.31082896 | 3.03E−12 |
| PDGFRA | −0.31213428 | 3.66E−09 |
| MYLIP | −0.31239862 | 0.00305231 |
| WNK1 | −0.31371948 | 2.85E−06 |
| RPL15 | −0.31381374 | 6.96E−58 |
| CPNE2 | −0.31450523 | 8.40E−11 |
| NSMCE1 | −0.31690314 | 4.08E−15 |
| ENSCAFG00000008336 | −0.31773946 | 7.54E−15 |
| S100A6 | −0.31834723 | 7.14E−29 |
| ENSCAFG00000029635 | −0.31853919 | 2.16E−27 |
| RPL23.1 | −0.31897707 | 4.47E−75 |
| ALDOC | −0.3202288 | 1.44E−05 |
| MET | −0.32041132 | 1.58E−12 |
| RPL28 | −0.32114441 | 1.45E−72 |
| MT-ND3 | −0.32117437 | 5.90E−24 |
| sept-11 | −0.32177457 | 5.49E−06 |
| ENSCAFG00000012725 | −0.3241188 | 9.34E−84 |
| ENSCAFG00000008776 | −0.32856626 | 4.48E−24 |
| CAV2 | −0.32918041 | 6.60E−21 |
| ENSCAFG00000012395 | −0.3294887 | 3.14E−79 |
| RACK1 | −0.33114297 | 7.96E−31 |
| FAIM | −0.33213103 | 8.41E−10 |
| PERP | −0.33359759 | 1.78E−15 |
| ENSCAFG00000012553 | −0.33360054 | 5.06E−07 |
| ENSCAFG00000006545 | −0.33425112 | 8.50E−24 |
| CDON | −0.3359286 | 5.67E−05 |
| DCXR | −0.33648006 | 3.65E−14 |
| ENSCAFG00000012676 | −0.33764386 | 8.57E−50 |
| RPS9 | −0.33797823 | 1.18E−68 |
| NR4A2 | −0.33836487 | 1 |
| EGLN1 | −0.33838955 | 1.26E−14 |
| RPL7A | −0.33906574 | 7.16E−43 |
| SERTAD4 | −0.34066495 | 4.26E−08 |
| RPL31 | −0.34097289 | 9.02E−43 |
| PRDX4 | −0.34132246 | 1.23E−05 |
| LSP1 | −0.34237651 | 4.73E−06 |
| PFDN5.1 | −0.34415804 | 3.86E−40 |
| THRAP3 | −0.34500485 | 2.12E−15 |
| SPTLC3 | −0.34527319 | 1.07E−06 |
| RPL27 | −0.34716721 | 6.72E−83 |
| COX7A1 | −0.3477697 | 1.62E−12 |
| ENSCAFG00000031952 | −0.34871773 | 1.69E−75 |
| ENSCAFG00000006734 | −0.34916722 | 4.98E−47 |
| CD59 | −0.34939808 | 2.99E−08 |
| ENSCAFG00000014648 | −0.35067979 | 1.17E−31 |
| TMA7 | −0.35455243 | 6.98E−28 |
| ENSCAFG00000000377 | −0.35513972 | 3.26E−65 |
| ENSCAFG00000008873 | −0.35553678 | 3.16E−29 |
| SSBP2 | −0.35603916 | 1.06E−16 |
| TSTD1 | −0.35753604 | 3.81E−20 |
| NAP1L1 | −0.35787215 | 2.53E−31 |
| ENSCAFG00000007199 | −0.35808819 | 1.69E−07 |
| IFI30 | −0.35851738 | 1.95E−16 |
| RPS18 | −0.35930671 | 1.63E−66 |
| ITM2C | −0.35992537 | 1.12E−11 |
| GLI3 | −0.36064372 | 9.71E−16 |
| RPS4X | −0.36073595 | 7.39E−68 |
| RPS17 | −0.36250787 | 2.54E−86 |
| MALL | −0.36321906 | 0.00544896 |
| NDUFB2 | −0.363249 | 1.47E−47 |
| ENSCAFG00000012839 | −0.36479984 | 2.36E−28 |
| ANLN | −0.36493066 | 4.03E−08 |
| NPNT | −0.3652741 | 0.00040752 |
| ENSCAFG00000033303 | −0.36765835 | 1.85E−11 |
| PTMA | −0.37007399 | 9.31E−13 |
| SSR4 | −0.37115863 | 1.71E−49 |
| ENSCAFG00000018187 | −0.37257065 | 9.37E−30 |
| CDKN2C | −0.37273569 | 3.27E−14 |
| ARID5B | −0.37401241 | 3.20E−10 |
| ENSCAFG00000032537 | −0.37489109 | 2.01E−29 |
| FTL | −0.37512085 | 6.70E−37 |
| RPL10A | −0.37521784 | 5.96E−69 |
| timp3 | −0.3754959 | 1.45E−06 |
| EFEMP1 | −0.37717726 | 1.66E−05 |
| BNIP3 | −0.37754141 | 3.43E−18 |
| NFIB | −0.37790027 | 3.52E−16 |
| ENSCAFG00000004260 | −0.38047277 | 4.07E−42 |
| ENSCAFG00000030466 | −0.38162162 | 1.15E−20 |
| KLF5 | −0.38266125 | 5.01E−10 |
| ENSCAFG00000015433 | −0.38290335 | 3.48E−35 |
| TRPS1 | −0.38607454 | 9.04E−23 |
| GLTSCR2 | −0.38646882 | 8.82E−17 |
| LRIG1 | −0.38741804 | 0.45650294 |
| RPL35 | −0.38837899 | 6.40E−96 |
| EPS8 | −0.38939014 | 1.05E−15 |
| ENSCAFG00000018784 | −0.39083723 | 3.47E−79 |
| RPS15A | −0.39507679 | 1.47E−95 |
| ENSCAFG00000016065 | −0.39527975 | 4.76E−29 |
| EIF3E | −0.39793997 | 4.14E−30 |
| SOX9 | −0.39832216 | 2.28E−15 |
| HSP90AB1 | −0.39919468 | 1.14E−23 |
| RPL7.1 | −0.40252969 | 9.64E−57 |
| USP6NL | −0.4044903 | 9.81E−09 |
| KARS | −0.40685681 | 4.09E−29 |
| ENSCAFG00000032152 | −0.40705689 | 2.87E−50 |
| FOS | −0.40760097 | 6.05E−12 |
| EIF3K | −0.40791741 | 1.51E−55 |
| ENSCAFG00000031706 | −0.40811952 | 5.87E−14 |
| HMGB2 | −0.41088923 | 3.15E−13 |
| CCND1 | −0.41110376 | 3.79E−12 |
| GM2A | −0.41336979 | 2.29E−12 |
| AIF1L | −0.41497493 | 1.01E−12 |
| ENSCAFG00000028936 | −0.41544652 | 1.22E−15 |
| RPS27 | −0.41766771 | 2.25E−84 |
| RDH10 | −0.41857351 | 1 |
| DBI | −0.42212407 | 1.39E−26 |
| EEF1D | −0.42420928 | 1.23E−48 |
| ENSCAFG00000031955 | −0.42524668 | 3.47E−101 |
| LIMCH1 | −0.42552716 | 1.02E−12 |
| ENSCAFG00000014958 | −0.42561197 | 1.06E−84 |
| PPP1R1B | −0.43481929 | 9.79E−16 |
| ENSCAFG00000029945 | −0.4358663 | 1.27E−51 |
| ABLIM1 | −0.43808576 | 4.33E−09 |
| MEI4 | −0.43823361 | 6.55E−65 |
| CST6 | −0.43891285 | 6.44E−21 |
| ENSCAFG00000028960 | −0.4393504 | 2.84E−44 |
| ENSCAFG00000012624 | −0.43958546 | 1.07E−36 |
| PPP1R3C | −0.4414792 | 2.49E−13 |
| ZFHX3 | −0.44526818 | 4.05E−21 |
| FRMD6 | −0.44801131 | 5.66E−19 |
| RPL3 | −0.44877661 | 3.98E−61 |
| ENSCAFG00000003564 | −0.4536463 | 1.24E−44 |
| DEK | −0.45464618 | 3.20E−21 |
| ENSCAFG00000032920 | −0.45475746 | 2.07E−27 |
| ZBTB16 | −0.45556596 | 6.23E−25 |
| ENSCAFG00000019044 | −0.46149169 | 4.79E−97 |
| INSIG1 | −0.46389424 | 2.41E−15 |
| FBXO2 | −0.46751861 | 2.22E−13 |
| IL18BP | −0.4702197 | 0.00212428 |
| EIF3L | −0.47603295 | 4.38E−32 |
| TFRC | −0.48601212 | 2.49E−21 |
| TSPAN13 | −0.48711431 | 7.58E−11 |
| PTGDR | −0.49035848 | 0.00147465 |
| BGLAP | −0.4908875 | 0.00032211 |
| GOLIM4 | −0.49288747 | 9.91E−13 |
| NUDT4 | −0.49350073 | 4.93E−15 |
| ENSCAFG00000032717 | −0.49606124 | 4.74E−93 |
| ZNF395 | −0.50102873 | 5.16E−29 |
| ENSCAFG00000024643 | −0.50180857 | 1.14E−49 |
| TSC22D3 | −0.50190377 | 2.67E−22 |
| CRYAB | −0.5056394 | 1.42E−14 |
| BBX | −0.50647686 | 6.73E−27 |
| ENSCAFG00000015834 | −0.51931737 | 3.02E−77 |
| ENSCAFG00000015372 | −0.51998521 | 3.15E−22 |
| BMP4 | −0.52039254 | 1 |
| SLC1A1 | −0.52843445 | 1.88E−10 |
| CADM1 | −0.5363386 | 3.68E−16 |
| ENSCAFG00000000027 | −0.5368636 | 3.09E−68 |
| AHNAK | −0.53702784 | 1.49E−15 |
| ENSCAFG00000017688 | −0.53835656 | 1.13E−100 |
| IMPDH2 | −0.5387333 | 2.55E−32 |
| FDFT1 | −0.55181825 | 3.95E−23 |
| TFAP2C | −0.55331187 | 8.72E−22 |
| ENSCAFG00000006884 | −0.55630239 | 6.04E−58 |
| APOE | −0.55635733 | 0.00074245 |
| MEF2C | −0.55718713 | 1.64E−21 |
| GSTM3 | −0.5694882 | 5.24E−14 |
| OGFRL1 | −0.57110992 | 4.48E−15 |
| TM4SF18 | −0.59863723 | 5.61E−19 |
| PRELP | −0.61360364 | 1.32E−22 |
| TNNI2 | −0.61963121 | 2.31E−18 |
| TGFBR3 | −0.62222398 | 4.53E−21 |
| IL33 | −0.627616 | 1.88E−13 |
| ABI3BP | −0.63172183 | 1.59E−14 |
| CLU | −0.63292644 | 1.21E−13 |
| SCG5 | −0.63496211 | 9.07E−22 |
| NDP | −0.64329834 | 4.58E−20 |
| ENSCAFG00000030307 | −0.64707163 | 7.11E−17 |
| ENSCAFG00000029553 | −0.66078747 | 1 |
| CTSD | −0.66916005 | 8.47E−14 |
| EIF4B | −0.68413781 | 7.28E−63 |
| EEF1A1 | −0.69294622 | 3.17E−104 |
| EDIL3 | −0.69733609 | 1.75E−28 |
| MGST1 | −0.72985887 | 3.99E−21 |
| B2M | −0.74207749 | 8.44E−26 |
| SHISA2 | −0.74376072 | 1.29E−24 |
| FGF7 | −0.74634343 | 3.26E−20 |
| FAM213B | −0.7710177 | 1.28E−22 |
| ENSCAFG00000018746 | −0.79340807 | 3.79E−22 |
| TCF7L2 | −0.85381978 | 1.01E−74 |
| ACAN | −0.87659982 | 9.99E−09 |
| ENSCAFG00000028765 | −0.93655397 | 3.09E−38 |
| MAL | −0.95458723 | 2.85E−22 |
| HAPLN1 | −0.98651343 | 1.02E−21 |
| FXYD6 | −0.98930748 | 2.20E−27 |
| FAM180A | −0.99904243 | 2.67E−19 |
| IFI6 | −1.00174878 | 6.45E−06 |
| ENSCAFG00000012963 | −1.04699494 | 1.30E−28 |
| CDO1 | −1.25074668 | 3.02E−17 |
| TF.1 | −1.25650717 | 1.71E−24 |
| SCRG1 | −1.30522628 | 1.67E−29 |
| C2orf40 | −1.34826821 | 2.43E−35 |
| COL2A1 | −1.59138813 | 8.90E−44 |

TABLE 3A

| Genes | avg_logFC | p_val_adj |
|---|---|---|
| SERPINE1 | 2.270134566 | 1.64E−124 |
| PLAU | 1.938321329 | 3.33E−101 |
| TF | 1.615323222 | 8.66E−70 |
| CYP1B1 | 1.605188409 | 3.59E−52 |
| PTGS2 | 1.601522321 | 2.60E−53 |
| CTGF | 1.594792721 | 2.67E−101 |
| GADD45B | 1.575014248 | 2.41E−111 |
| PMEPA1 | 1.368238395 | 1.77E−138 |
| RGS2 | 1.33586132 | 3.82E−30 |
| LIF | 1.185820503 | 2.35E−68 |
| ENSCAFG00000007045 | 1.127510697 | 4.59E−56 |
| PLAUR | 1.080355294 | 2.92E−83 |
| BHLHE40 | 0.987427044 | 2.44E−75 |
| STC2 | 0.973849543 | 1.62E−60 |
| GDNF | 0.951146608 | 1.94E−65 |
| INHBA | 0.938581446 | 1.09E−42 |
| FSTL3 | 0.915068598 | 1.25E−68 |
| ALAS1 | 0.910462856 | 9.86E−46 |
| DDX58 | 0.907727781 | 7.51E−05 |
| SNAI1 | 0.880863852 | 3.42E−59 |
| HAS2 | 0.831263218 | 1.42E−38 |
| CYR61 | 0.822041938 | 1.81E−55 |
| VCAN | 0.791744247 | 3.50E−53 |
| HBEGF | 0.743209509 | 3.29E−31 |
| LTBP1 | 0.726556801 | 6.54E−43 |
| UGDH | 0.723736501 | 2.68E−54 |
| SKIL | 0.722523271 | 1.13E−68 |
| ENSCAFG00000032813 | 0.69050212 | 2.99E−22 |
| CYP26B1 | 0.66026285 | 7.51E−06 |
| EDN1 | 0.659509753 | 1.23E−11 |
| FST | 0.649885638 | 1.53E−06 |
| RASL11A | 0.643700697 | 1 |
| PTHLH | 0.632177886 | 5.00E−13 |
| TFPI2 | 0.631465532 | 3.09E−31 |
| BAZ1A | 0.625318472 | 3.17E−59 |
| LRRC8C | 0.613489029 | 3.03E−43 |
| PDPN | 0.612788854 | 1.17E−25 |
| ISG20 | 0.610179996 | 1.71E−28 |
| ACTN1 | 0.607997286 | 7.22E−54 |
| THBS1 | 0.572688209 | 1.13E−34 |
| ENSCAFG00000015625 | 0.569353099 | 4.40E−22 |
| ENSCAFG00000002015 | 0.566931371 | 2.05E−46 |
| CYCS | 0.565808469 | 3.55E−63 |
| PDE4B | 0.564477965 | 1.51E−27 |
| COL5A1 | 0.55491057 | 6.15E−27 |
| PDLIM5 | 0.55338401 | 5.05E−42 |
| ELOVL4 | 0.541004424 | 8.53E−32 |
| ICAM1 | 0.540189379 | 1.40E−31 |
| FOXP4 | 0.538976974 | 5.14E−38 |
| NGF | 0.536034633 | 7.86E−25 |
| RBMS1 | 0.520889735 | 1.39E−56 |
| ACTA2 | 0.51835637 | 4.77E−05 |
| ENSCAFG00000033998 | 0.510076931 | 2.01E−21 |
| ADSS | 0.508717264 | 2.27E−29 |
| WISP1 | 0.505862364 | 1.47E−14 |
| ELK3 | 0.505306706 | 4.15E−28 |
| MAP1B | 0.501889012 | 3.96E−29 |
| S100A2 | 0.501136569 | 1 |
| MARCH3 | 0.495772749 | 1.36E−25 |
| NF2 | 0.494490521 | 4.59E−16 |
| ENSCAFG00000003758 | 0.49399392 | 1.14E−30 |
| SNRNP48 | 0.489452297 | 3.82E−37 |
| NIPAL1 | 0.488938551 | 9.16E−25 |
| TNFSF10 | 0.487114772 | 1.26E−27 |
| MT2A | 0.484966088 | 9.09E−27 |
| MEDAG | 0.483035018 | 5.29E−14 |
| COL1A1 | 0.481300099 | 2.93E−14 |
| IFRD1 | 0.47940483 | 1.85E−26 |
| DNAJB4 | 0.478466459 | 2.86E−25 |
| KCTD10 | 0.472668513 | 7.71E−34 |
| RCAN1 | 0.467451478 | 2.95E−19 |
| PALLD | 0.46704451 | 3.09E−25 |
| FZD2 | 0.461441264 | 3.70E−42 |
| KIF5B | 0.452934638 | 9.92E−51 |
| FN1 | 0.451706657 | 7.67E−21 |
| ENSCAFG00000006436 | 0.450184308 | 9.50E−28 |
| GLRX | 0.447673859 | 7.04E−08 |
| VPS37A | 0.44658027 | 6.47E−31 |
| SLC4A7 | 0.44656017 | 8.43E−31 |
| ENSCAFG00000037735 | 0.44491503 | 9.27E−20 |
| NEK7 | 0.440671399 | 1.16E−23 |
| ENSCAFG00000024171 | 0.439808333 | 2.72E−13 |
| PLOD2 | 0.435799011 | 5.58E−32 |
| SSX2IP | 0.433433068 | 5.40E−22 |
| HIP1 | 0.430291869 | 2.35E−28 |
| ELL2 | 0.429880706 | 4.21E−26 |
| PTK7 | 0.428408906 | 2.31E−26 |
| ANO6 | 0.42819938 | 6.54E−27 |
| ETS1 | 0.42651363 | 1.16E−23 |
| COL8A1 | 0.425783098 | 1.12E−05 |
| IQCJ-SCHIP1 | 0.421507132 | 2.17E−15 |
| LOX | 0.421443873 | 1.73E−18 |
| SH3BP5L | 0.420108354 | 8.54E−23 |
| RAI14 | 0.418838543 | 5.16E−24 |
| SNX10 | 0.416905955 | 2.20E−24 |
| ADAM19 | 0.416225598 | 7.29E−18 |
| COL12A1 | 0.415635917 | 3.05E−22 |
| HSPH1 | 0.412816956 | 6.08E−27 |
| SRPX2 | 0.412666738 | 1.53E−24 |
| DLC1 | 0.411009682 | 2.22E−19 |
| ALDH18A1 | 0.410604989 | 6.49E−33 |
| AMOTL2 | 0.410370891 | 1.12E−18 |
| SDC4 | 0.406600535 | 1.73E−09 |
| PFKFB3 | 0.403011464 | 1 |
| NRP2 | 0.402252102 | 9.73E−24 |
| ENSCAFG00000000345 | 0.398102466 | 6.59E−26 |
| CRABP2 | 0.396979592 | 2.93E−14 |
| GORAB | 0.396186256 | 1.23E−15 |
| TPM2 | 0.394423601 | 0.0010039 |
| LUM | 0.392747857 | 2.43E−05 |
| ENSCAFG00000013979 | 0.388719855 | 3.97E−14 |
| SPHK1 | 0.38692647 | 4.99E−28 |
| IFIH1 | 0.38656085 | 7.45E−06 |
| COL3A1 | 0.383267542 | 4.72E−13 |
| BGN | 0.383210386 | 5.66E−14 |
| PICALM | 0.382048887 | 1.62E−29 |
| RALA | 0.377466022 | 4.13E−22 |
| ITGA5 | 0.372426688 | 0.00022458 |
| ZNFX1 | 0.371873777 | 2.03E−23 |
| ENSCAFG00000036684 | 0.371392695 | 1.83E−17 |
| MAP7D1 | 0.367099126 | 8.37E−18 |
| SRM | 0.366802221 | 1.71E−19 |
| BCL2L11 | 0.366623544 | 1.54E−14 |
| AEN | 0.366556769 | 4.58E−09 |
| MYH9 | 0.365525825 | 1.82E−23 |
| YIF1B | 0.364924894 | 5.58E−20 |
| NFKBIA | 0.363974328 | 0.00098373 |
| PHF20L1 | 0.363486584 | 5.95E−20 |
| PDZRN3 | 0.361763681 | 3.68E−24 |
| BLOC1S2 | 0.360110531 | 8.67E−25 |
| GTF2F1 | 0.359984498 | 7.39E−18 |
| PTGES | 0.354877191 | 1 |
| SPOP | 0.354769454 | 3.71E−17 |
| BCAR1 | 0.354670991 | 5.43E−18 |
| GFPT2 | 0.353801801 | 3.84E−08 |
| CKAP4 | 0.353447818 | 6.34E−26 |
| ENSCAFG00000031852 | 0.351467487 | 9.04E−27 |
| TUBA4A | 0.351136866 | 3.09E−12 |
| PNPLA2 | 0.351032625 | 5.69E−18 |
| CTNNB1 | 0.348949126 | 7.58E−35 |
| FAS | 0.348560418 | 7.60E−17 |
| SYNC | 0.346548987 | 2.88E−07 |
| MMD | 0.345335146 | 1.13E−15 |
| TCF4 | 0.34342355 | 2.19E−13 |
| TCIRG1 | 0.341695915 | 1.25E−15 |
| IBTK | 0.341267978 | 1.95E−18 |
| GOLT1B | 0.340457605 | 3.76E−21 |
| SNRNP40 | 0.34009679 | 1.00E−17 |
| COL1A2 | 0.339411575 | 2.05E−10 |
| HAPLN3 | 0.33610249 | 5.47E−13 |
| SACS | 0.33507603 | 4.06E−13 |
| PINX1 | 0.334967829 | 2.96E−13 |
| CDK8 | 0.334822383 | 1.34E−18 |
| ENSCAFG00000011482 | 0.332844714 | 2.69E−14 |
| UBE2J1 | 0.332459 | 8.26E−16 |
| S100A4 | 0.331511753 | 4.16E−05 |
| STARD13 | 0.330863114 | 7.13E−12 |
| SRSF7 | 0.330639938 | 2.33E−16 |
| MPP6 | 0.330320089 | 1.35E−13 |
| STK38L | 0.330194066 | 1.22E−11 |
| VEGFA | 0.326552564 | 7.11E−07 |
| GNL3 | 0.326489092 | 1.78E−15 |
| COL5A2 | 0.325283785 | 1.90E−16 |
| PID1 | 0.324789537 | 5.20E−07 |
| NUAK1 | 0.322608179 | 7.38E−12 |
| LMCD1 | 0.322505239 | 0.00031673 |
| LPCAT2 | 0.321883143 | 2.06E−12 |
| THBS2 | 0.321515274 | 9.22E−06 |
| ENSCAFG00000037410 | 0.321415081 | 1.18E−16 |
| ATP13A3 | 0.320744061 | 8.61E−15 |
| ANKRD10 | 0.320687221 | 2.04E−14 |
| TMEM2 | 0.320271599 | 6.72E−11 |
| ENSCAFG00000018440 | 0.318205368 | 3.56E−12 |
| REL | 0.316388368 | 3.30E−17 |
| SNAI2 | 0.316301052 | 1 |
| ENSCAFG00000001840 | 0.315952691 | 6.09E−13 |
| TXNRD1 | 0.314990868 | 2.15E−18 |
| RIC1 | 0.31260878 | 2.06E−14 |
| SMC5 | 0.312166654 | 8.07E−13 |
| NDUFAF4 | 0.312125069 | 1.97E−13 |
| NOP58 | 0.312027948 | 1.77E−09 |
| CSRP1 | 0.311483153 | 1.84E−23 |
| TMEM41B | 0.311216589 | 2.61E−12 |
| ENSCAFG00000017326 | 0.309316441 | 0.65348525 |
| NUDC | 0.308853972 | 1.66E−19 |
| STAM2 | 0.308195198 | 4.20E−20 |
| ENSCAFG00000001448 | 0.307218421 | 4.61E−30 |
| MYLK | 0.307130976 | 2.61E−09 |
| ACVR1 | 0.30491048 | 5.65E−13 |
| RNF6 | 0.304133178 | 1.63E−16 |
| CCT4 | 0.303922731 | 2.48E−17 |
| FAM49B | 0.302795732 | 1.45E−13 |
| TKT | 0.302581621 | 8.90E−06 |
| PCSK1 | 0.302052584 | 1.88E−31 |
| SMG7 | 0.301762461 | 1.82E−12 |
| MRPS31 | 0.301222195 | 6.87E−19 |
| TUBA1C | 0.300991012 | 2.83E−10 |
| UBE2S | 0.300842341 | 1.06E−13 |
| NUDCD1 | 0.29796364 | 2.49E−14 |
| CRLF3 | 0.297570697 | 9.93E−15 |
| TWF1 | 0.296148932 | 6.20E−16 |
| DDX60 | 0.295595225 | 1 |
| GTF2H1 | 0.29497148 | 3.45E−15 |
| FAM126A | 0.294401112 | 3.95E−12 |
| PNPLA8 | 0.292016173 | 1.55E−15 |
| FAM20C | 0.291791458 | 2.00E−09 |
| MT1 | 0.290657433 | 0.14826836 |
| DNAJB9 | 0.289623435 | 2.82E−12 |
| ENSCAFG00000029851 | 0.289545577 | 3.15E−14 |
| PRPF38B | 0.288814072 | 1.03E−13 |
| RPS6KC1 | 0.288104439 | 1.19E−14 |
| DKK1 | 0.288032443 | 3.79E−07 |
| ENSCAFG00000030449 | 0.285576385 | 5.59E−07 |
| DPP4 | 0.285457297 | 2.29E−11 |
| FLNB | 0.284752033 | 1.74E−16 |
| PLS3 | 0.284373241 | 5.79E−12 |
| CLCF1 | 0.284081513 | 4.42E−24 |
| ATP6V1H | 0.280867753 | 4.10E−12 |
| GPATCH4 | 0.280788015 | 3.35E−09 |
| ENSCAFG00000007306 | 0.279060899 | 1.93E−13 |
| TMF1 | 0.278899959 | 4.01E−12 |
| MAOA | 0.277829558 | 1.24E−11 |
| NAV3 | 0.277094704 | 1.59E−10 |
| SOD2 | 0.276366887 | 1.30E−12 |
| EMD | 0.276004674 | 1.41E−10 |
| ANKRD37 | 0.275977979 | 1 |
| PLK2 | 0.274743272 | 2.22E−13 |
| NEXN | 0.27437062 | 8.54E−11 |
| SEC24D | 0.27395841 | 8.01E−12 |
| C11orf24 | 0.27329155 | 5.01E−10 |
| C10orf90 | 0.273012658 | 7.82E−14 |
| NRBF2 | 0.272928691 | 4.97E−11 |
| YRDC | 0.272465098 | 6.79E−11 |
| ADAM12 | 0.272089054 | 3.81E−11 |
| DSE | 0.271615184 | 9.19E−10 |
| GLO1 | 0.269818214 | 1.01E−13 |
| WNT9A | 0.269258272 | 5.10E−17 |
| TMEM218 | 0.268923991 | 1.14E−08 |
| BNC1 | 0.268795705 | 1.05E−22 |
| GPNMB | 0.267056623 | 0.01187383 |
| ENSCAFG00000012041 | 0.266965762 | 1.07E−10 |
| PPP1R15B | 0.266453221 | 1.78E−10 |
| SEMA3C | 0.265622693 | 5.19E−08 |
| STXBP6 | 0.265520629 | 4.60E−08 |
| PTPN12 | 0.265435261 | 4.09E−11 |
| SRSF11 | 0.265125218 | 4.34E−13 |
| MAPK8 | 0.265047739 | 8.76E−12 |
| UGCG | 0.264965223 | 4.27E−06 |
| MSN | 0.264350131 | 5.07E−14 |
| ACTG2 | 0.264339894 | 1 |
| FADS1 | 0.263944354 | 3.63E−06 |
| SLC25A16 | 0.263112844 | 3.93E−11 |
| TIMM8A | 0.262918333 | 7.11E−10 |
| ENSCAFG00000016011 | 0.262799466 | 1.59E−11 |
| SLC20A1 | 0.261386402 | 8.28E−09 |
| PHAX | 0.259855919 | 4.67E−12 |
| FAM46A | 0.259808127 | 0.00076126 |
| C1orf52 | 0.258140183 | 9.52E−09 |
| SNX16 | 0.257966718 | 5.26E−09 |
| STX7 | 0.257533159 | 6.61E−10 |
| EIF1AD | 0.257048668 | 6.20E−12 |
| ENSCAFG00000002833 | 0.256902211 | 1.84E−10 |
| LGALS3 | 0.25589799 | 2.41E−12 |
| ENSCAFG00000009110 | 0.255855497 | 0.00319632 |
| PA2G4 | 0.255149397 | 9.63E−09 |
| DNAJC25 | 0.255086843 | 2.37E−11 |
| MYBL1 | 0.255034351 | 8.97E−07 |
| TGIF2-C20orf24 | 0.254704432 | 4.74E−24 |
| MZT1 | 0.253444595 | 2.30E−19 |
| EGR1 | 0.253387664 | 0.00017755 |
| ABRACL | 0.253085413 | 2.76E−09 |
| DUS4L | 0.253085413 | 1.08E−12 |
| WDR70 | 0.253085413 | 1.20E−11 |
| ENSCAFG00000006693 | 0.252862024 | 1.44E−12 |
| ENSCAFG00000015396 | 0.252221482 | 9.63E−11 |
| MYO1E | 0.251784176 | 2.76E−08 |
| ALG5 | 0.251159176 | 6.87E−13 |
| SYTL4 | 0.250850775 | 1.78E−08 |
| HAUS3 | 0.250669369 | 7.79E−12 |
| LIN7B | 0.250504766 | 9.14E−07 |
| ENSCAFG00000019620 | 0.250464179 | 4.76E−07 |
| ND4L | −0.2502282 | 1.22E−11 |
| ENSCAFG00000006810 | −0.2504346 | 1.18E−35 |
| ENSCAFG00000001968 | −0.25169181 | 3.34E−09 |
| ENSCAFG00000029055 | −0.25234755 | 1.34E−11 |
| KIAA1217 | −0.2529506 | 0.00179526 |
| ENSCAFG00000023205 | −0.25319681 | 1.13E−09 |
| SLC38A2 | −0.25463943 | 3.95E−09 |
| RPL27A | −0.25527852 | 6.77E−19 |
| ATP5F1 | −0.25558736 | 2.46E−12 |
| SYNE2 | −0.25563906 | 0.00564005 |
| FTH1.1 | −0.25601459 | 1.65E−28 |
| IRX2 | −0.25697068 | 4.15E−07 |
| RPS23 | −0.25707733 | 3.24E−66 |
| PLEKHA2 | −0.25732945 | 4.85E−10 |
| TMSB10 | −0.25738773 | 1.06E−07 |
| EFEMP2 | −0.25774021 | 2.43E−06 |
| RPL32 | −0.2580165 | 1.35E−68 |
| RPS16 | −0.26051969 | 6.36E−53 |
| OLFM1 | −0.26165411 | 5.40E−06 |
| MATN2 | −0.26173223 | 0.22784127 |
| CA9 | −0.26259303 | 0.00077323 |
| ENSCAFG00000031486 | −0.26282112 | 3.03E−12 |
| ENSCAFG00000031458 | −0.26366542 | 6.53E−27 |
| EIF3G | −0.2640728 | 1.09E−10 |
| RPS8 | −0.26452057 | 5.76E−52 |
| ENSCAFG00000013201 | −0.26458174 | 1.98E−09 |
| NCBP2 | −0.26478778 | 1.63E−14 |
| IRF3 | −0.26479261 | 2.42E−05 |
| COL11A1 | −0.26495472 | 2.11E−07 |
| ATP5J | −0.26513943 | 7.57E−36 |
| ENSCAFG00000001214 | −0.26698805 | 4.12E−43 |
| HAGH | −0.26721896 | 4.44E−05 |
| SNRPG | −0.26737977 | 1.23E−28 |
| RPS27A | −0.267553 | 7.06E−50 |
| PLA2G7 | −0.26798435 | 8.00E−05 |
| COX7A2 | −0.26828031 | 2.01E−33 |
| CRYL1 | −0.26869155 | 0.03987256 |
| UQCC2 | −0.26872058 | 3.03E−20 |
| RPS19 | −0.26965853 | 2.54E−54 |
| LSM8 | −0.27001268 | 1.71E−17 |
| ENSCAFG00000033694 | −0.27033944 | 1.27E−05 |
| NAA38 | −0.27091997 | 1.03E−13 |
| ENSCAFG00000013214 | −0.27116323 | 0.0001272 |
| ENSCAFG00000015445 | −0.27222156 | 3.77E−14 |
| EIF3H | −0.27225303 | 5.17E−15 |
| FAM162A | −0.27255667 | 5.10E−19 |
| ENSCAFG00000031671 | −0.27326291 | 1.45E−12 |
| DHRS7 | −0.27358298 | 1.57E−13 |
| ENSCAFG00000009523 | −0.27388325 | 3.12E−65 |
| ENSCAFG00000024837 | −0.27503938 | 1.26E−06 |
| CAV1 | −0.27575872 | 1.13E−08 |
| PIK3R1 | −0.27616776 | 6.20E−08 |
| PNP | −0.27624136 | 1.22E−10 |
| RPS3 | −0.27646423 | 4.97E−41 |
| PRDX6 | −0.27721779 | 2.74E−10 |
| SYNE1 | −0.27766239 | 1.12E−06 |
| TGFB2 | −0.27860449 | 1.20E−05 |
| C19orf70 | −0.27913707 | 1.68E−16 |
| RPL9 | −0.27933313 | 3.27E−56 |
| UBA52 | −0.27967552 | 1.06E−61 |
| IRX3 | −0.27994437 | 2.12E−12 |
| ENSCAFG00000000567 | −0.28048729 | 3.78E−32 |
| SIX1 | −0.28063403 | 1.81E−08 |
| ENSCAFG00000031583 | −0.2822734 | 5.57E−05 |
| COL8A2 | −0.28259098 | 2.47E−06 |
| TMEM47 | −0.28263776 | 7.01E−06 |
| RPS14 | −0.28359277 | 3.94E−64 |
| ENSCAFG00000024413 | −0.28407068 | 1.57E−22 |
| EIF4A2 | −0.28532648 | 2.77E−12 |
| RPS15 | −0.28589156 | 1.15E−73 |
| PTRHD1 | −0.28636413 | 2.85E−09 |
| SCARB1 | −0.28673244 | 0.00013137 |
| RPL5 | −0.28692681 | 1.38E−38 |
| MRPL36 | −0.28821789 | 2.66E−19 |
| UQCRQ | −0.28822847 | 3.47E−22 |
| TMEM106C | −0.28847789 | 9.60E−10 |
| NUDT2 | −0.28939319 | 1.74E−10 |
| ENSCAFG00000000061 | −0.28999226 | 3.63E−16 |
| HP1BP3 | −0.29072458 | 3.45E−10 |
| COX4I1 | −0.29091796 | 1.88E−26 |
| MINOS1 | −0.29188549 | 7.83E−13 |
| RPS6 | −0.29213686 | 4.42E−59 |
| ENSCAFG00000007990 | −0.29262724 | 4.16E−53 |
| ENSCAFG00000014234 | −0.29369794 | 3.16E−14 |
| DDOST | −0.29460599 | 4.75E−12 |
| NDUFS5 | −0.29480992 | 3.04E−14 |
| ENSCAFG00000025332 | −0.29552836 | 3.73E−08 |
| OCIAD2 | −0.29661566 | 6.10E−05 |
| RPL23 | −0.29704381 | 4.23E−14 |
| PLCD1 | −0.29814697 | 9.30E−09 |
| ENSCAFG00000008935 | −0.29847055 | 3.16E−16 |
| RPS5 | −0.29888365 | 8.74E−49 |
| KRTCAP2 | −0.29965666 | 3.53E−32 |
| RPS7 | −0.3001245 | 3.11E−61 |
| ADGRG1 | −0.30027415 | 8.02E−07 |
| AK1 | −0.3003055 | 1.74E−17 |
| ETV5 | −0.30032907 | 0.00065529 |
| CUTA | −0.30090167 | 2.16E−15 |
| MACF1 | −0.3025817 | 2.22E−14 |
| S100A13 | −0.30389326 | 3.77E−21 |
| RPL11 | −0.3055851 | 1.77E−61 |
| KIAA1551 | −0.30634507 | 7.15E−05 |
| DDX41 | −0.30678774 | 1.74E−09 |
| ENSCAFG00000010850 | −0.30709786 | 7.99E−21 |
| ENSCAFG00000004800 | −0.30879851 | 8.64E−20 |
| RPL14 | −0.30950893 | 3.75E−53 |
| ATPIF1 | −0.31009434 | 1.05E−30 |
| ENSCAFG00000024445 | −0.31072658 | 8.93E−17 |
| ENSCAFG00000019141 | −0.31082896 | 3.03E−12 |
| PDGFRA | −0.31213428 | 3.66E−09 |
| MYLIP | −0.31239862 | 0.00305231 |
| WNK1 | −0.31371948 | 2.85E−06 |
| RPL15 | −0.31381374 | 6.96E−58 |
| CPNE2 | −0.31450523 | 8.40E−11 |
| NSMCE1 | −0.31690314 | 4.08E−15 |
| ENSCAFG00000008336 | −0.31773946 | 7.54E−15 |
| S100A6 | −0.31834723 | 7.14E−29 |
| ENSCAFG00000029635 | −0.31853919 | 2.16E−27 |
| RPL23.1 | −0.31897707 | 4.47E−75 |
| ALDOC | −0.3202288 | 1.44E−05 |
| MET | −0.32041132 | 1.58E−12 |
| RPL28 | −0.32114441 | 1.45E−72 |
| MT-ND3 | −0.32117437 | 5.90E−24 |
| sept-11 | −0.32177457 | 5.49E−06 |
| ENSCAFG00000012725 | −0.3241188 | 9.34E−84 |
| ENSCAFG00000008776 | −0.32856626 | 4.48E−24 |
| CAV2 | −0.32918041 | 6.60E−21 |
| ENSCAFG00000012395 | −0.3294887 | 3.14E−79 |
| RACK1 | −0.33114297 | 7.96E−31 |
| FAIM | −0.33213103 | 8.41E−10 |
| PERP | −0.33359759 | 1.78E−15 |
| ENSCAFG00000012553 | −0.33360054 | 5.06E−07 |
| ENSCAFG00000006545 | −0.33425112 | 8.50E−24 |
| CDON | −0.3359286 | 5.67E−05 |
| DCXR | −0.33648006 | 3.65E−14 |
| ENSCAFG00000012676 | −0.33764386 | 8.57E−50 |
| RPS9 | −0.33797823 | 1.18E−68 |
| NR4A2 | −0.33836487 | 1 |
| EGLN1 | −0.33838955 | 1.26E−14 |
| RPL7A | −0.33906574 | 7.16E−43 |
| SERTAD4 | −0.34066495 | 4.26E−08 |
| RPL31 | −0.34097289 | 9.02E−43 |
| PRDX4 | −0.34132246 | 1.23E−05 |
| LSP1 | −0.34237651 | 4.73E−06 |
| PFDN5.1 | −0.34415804 | 3.86E−40 |
| THRAP3 | −0.34500485 | 2.12E−15 |
| SPTLC3 | −0.34527319 | 1.07E−06 |
| RPL27 | −0.34716721 | 6.72E−83 |
| COX7A1 | −0.3477697 | 1.62E−12 |
| ENSCAFG00000031952 | −0.34871773 | 1.69E−75 |
| ENSCAFG00000006734 | −0.34916722 | 4.98E−47 |
| CD59 | −0.34939808 | 2.99E−08 |
| ENSCAFG00000014648 | −0.35067979 | 1.17E−31 |
| TMA7 | −0.35455243 | 6.98E−28 |
| ENSCAFG00000000377 | −0.35513972 | 3.26E−65 |
| ENSCAFG00000008873 | −0.35553678 | 3.16E−29 |
| SSBP2 | −0.35603916 | 1.06E−16 |
| TSTD1 | −0.35753604 | 3.81E−20 |
| NAP1L1 | −0.35787215 | 2.53E−31 |
| ENSCAFG00000007199 | −0.35808819 | 1.69E−07 |
| IFI30 | −0.35851738 | 1.95E−16 |
| RPS18 | −0.35930671 | 1.63E−66 |
| ITM2C | −0.35992537 | 1.12E−11 |
| GLI3 | −0.36064372 | 9.71E−16 |
| RPS4X | −0.36073595 | 7.39E−68 |
| RPS17 | −0.36250787 | 2.54E−86 |
| MALL | −0.36321906 | 0.00544896 |
| NDUFB2 | −0.363249 | 1.47E−47 |
| ENSCAFG00000012839 | −0.36479984 | 2.36E−28 |
| ANLN | −0.36493066 | 4.03E−08 |
| NPNT | −0.3652741 | 0.00040752 |
| ENSCAFG00000033303 | −0.36765835 | 1.85E−11 |
| PTMA | −0.37007399 | 9.31E−13 |
| SSR4 | −0.37115863 | 1.71E−49 |
| ENSCAFG00000018187 | −0.37257065 | 9.37E−30 |
| CDKN2C | −0.37273569 | 3.27E−14 |
| ARID5B | −0.37401241 | 3.20E−10 |
| ENSCAFG00000032537 | −0.37489109 | 2.01E−29 |
| FTL | −0.37512085 | 6.70E−37 |
| RPL10A | −0.37521784 | 5.96E−69 |
| timp3 | −0.3754959 | 1.45E−06 |
| EFEMP1 | −0.37717726 | 1.66E−05 |
| BNIP3 | −0.37754141 | 3.43E−18 |
| NFIB | −0.37790027 | 3.52E−16 |
| ENSCAFG00000004260 | −0.38047277 | 4.07E−42 |
| ENSCAFG00000030466 | −0.38162162 | 1.15E−20 |
| KLF5 | −0.38266125 | 5.01E−10 |
| ENSCAFG00000015433 | −0.38290335 | 3.48E−35 |
| TRPS1 | −0.38607454 | 9.04E−23 |
| GLTSCR2 | −0.38646882 | 8.82E−17 |
| LRIG1 | −0.38741804 | 0.45650294 |
| RPL35 | −0.38837899 | 6.40E−96 |
| EPS8 | −0.38939014 | 1.05E−15 |
| ENSCAFG00000018784 | −0.39083723 | 3.47E−79 |
| RPS15A | −0.39507679 | 1.47E−95 |
| ENSCAFG00000016065 | −0.39527975 | 4.76E−29 |
| EIF3E | −0.39793997 | 4.14E−30 |
| SOX9 | −0.39832216 | 2.28E−15 |
| HSP90AB1 | −0.39919468 | 1.14E−23 |
| RPL7.1 | −0.40252969 | 9.64E−57 |
| USP6NL | −0.4044903 | 9.81E−09 |
| KARS | −0.40685681 | 4.09E−29 |
| ENSCAFG00000032152 | −0.40705689 | 2.87E−50 |
| FOS | −0.40760097 | 6.05E−12 |
| EIF3K | −0.40791741 | 1.51E−55 |
| ENSCAFG00000031706 | −0.40811952 | 5.87E−14 |
| HMGB2 | −0.41088923 | 3.15E−13 |
| CCND1 | −0.41110376 | 3.79E−12 |
| GM2A | −0.41336979 | 2.29E−12 |
| AIF1L | −0.41497493 | 1.01E−12 |
| ENSCAFG00000028936 | −0.41544652 | 1.22E−15 |
| RPS27 | −0.41766771 | 2.25E−84 |
| RDH10 | −0.41857351 | 1 |
| DBI | −0.42212407 | 1.39E−26 |
| EEF1D | −0.42420928 | 1.23E−48 |
| ENSCAFG00000031955 | −0.42524668 | 3.47E−101 |
| LIMCH1 | −0.42552716 | 1.02E−12 |
| ENSCAFG00000014958 | −0.42561197 | 1.06E−84 |
| PPP1R1B | −0.43481929 | 9.79E−16 |
| ENSCAFG00000029945 | −0.4358663 | 1.27E−51 |
| ABLIM1 | −0.43808576 | 4.33E−09 |
| MEI4 | −0.43823361 | 6.55E−65 |
| CST6 | −0.43891285 | 6.44E−21 |
| ENSCAFG00000028960 | −0.4393504 | 2.84E−44 |
| ENSCAFG00000012624 | −0.43958546 | 1.07E−36 |
| PPP1R3C | −0.4414792 | 2.49E−13 |
| ZFHX3 | −0.44526818 | 4.05E−21 |
| FRMD6 | −0.44801131 | 5.66E−19 |
| RPL3 | −0.44877661 | 3.98E−61 |
| ENSCAFG00000003564 | −0.4536463 | 1.24E−44 |
| DEK | −0.45464618 | 3.20E−21 |
| ENSCAFG00000032920 | −0.45475746 | 2.07E−27 |
| ZBTB16 | −0.45556596 | 6.23E−25 |
| ENSCAFG00000019044 | −0.46149169 | 4.79E−97 |
| INSIG1 | −0.46389424 | 2.41E−15 |
| FBXO2 | −0.46751861 | 2.22E−13 |
| IL18BP | −0.4702197 | 0.00212428 |
| EIF3L | −0.47603295 | 4.38E−32 |
| TFRC | −0.48601212 | 2.49E−21 |
| TSPAN13 | −0.48711431 | 7.58E−11 |
| PTGDR | −0.49035848 | 0.00147465 |
| BGLAP | −0.4908875 | 0.00032211 |
| GOLIM4 | −0.49288747 | 9.91E−13 |
| NUDT4 | −0.49350073 | 4.93E−15 |
| ENSCAFG00000032717 | −0.49606124 | 4.74E−93 |
| ZNF395 | −0.50102873 | 5.16E−29 |
| ENSCAFG00000024643 | −0.50180857 | 1.14E−49 |
| TSC22D3 | −0.50190377 | 2.67E−22 |
| CRYAB | −0.5056394 | 1.42E−14 |
| BBX | −0.50647686 | 6.73E−27 |
| ENSCAFG00000015834 | −0.51931737 | 3.02E−77 |
| ENSCAFG00000015372 | −0.51998521 | 3.15E−22 |
| BMP4 | −0.52039254 | 1 |
| SLC1A1 | −0.52843445 | 1.88E−10 |
| CADM1 | −0.5363386 | 3.68E−16 |
| ENSCAFG00000000027 | −0.5368636 | 3.09E−68 |
| AHNAK | −0.53702784 | 1.49E−15 |
| ENSCAFG00000017688 | −0.53835656 | 1.13E−100 |
| IMPDH2 | −0.5387333 | 2.55E−32 |
| FDFT1 | −0.55181825 | 3.95E−23 |
| TFAP2C | −0.55331187 | 8.72E−22 |
| ENSCAFG00000006884 | −0.55630239 | 6.04E−58 |
| APOE | −0.55635733 | 0.00074245 |
| MEF2C | −0.55718713 | 1.64E−21 |
| GSTM3 | −0.5694882 | 5.24E−14 |
| OGFRL1 | −0.57110992 | 4.48E−15 |
| TM4SF18 | −0.59863723 | 5.61E−19 |
| PRELP | −0.61360364 | 1.32E−22 |
| TNNI2 | −0.61963121 | 2.31E−18 |
| TGFBR3 | −0.62222398 | 4.53E−21 |
| IL33 | −0.627616 | 1.88E−13 |
| ABI3BP | −0.63172183 | 1.59E−14 |
| CLU | −0.63292644 | 1.21E−13 |
| SCG5 | −0.63496211 | 9.07E−22 |
| NDP | −0.64329834 | 4.58E−20 |
| ENSCAFG00000030307 | −0.64707163 | 7.11E−17 |
| ENSCAFG00000029553 | −0.66078747 | 1 |
| CTSD | −0.66916005 | 8.47E−14 |
| EIF4B | −0.68413781 | 7.28E−63 |
| EEF1A1 | −0.69294622 | 3.17E−104 |
| EDIL3 | −0.69733609 | 1.75E−28 |
| MGST1 | −0.72985887 | 3.99E−21 |
| B2M | −0.74207749 | 8.44E−26 |
| SHISA2 | −0.74376072 | 1.29E−24 |
| FGF7 | −0.74634343 | 3.26E−20 |
| FAM213B | −0.7710177 | 1.28E−22 |
| ENSCAFG00000018746 | −0.79340807 | 3.79E−22 |
| TCF7L2 | −0.85381978 | 1.01E−74 |
| ACAN | −0.87659982 | 9.99E−09 |
| ENSCAFG00000028765 | −0.93655397 | 3.09E−38 |
| MAL | −0.95458723 | 2.85E−22 |
| HAPLN1 | −0.98651343 | 1.02E−21 |
| FXYD6 | −0.98930748 | 2.20E−27 |
| FAM180A | −0.99904243 | 2.67E−19 |
| IFI6 | −1.00174878 | 6.45E−06 |
| ENSCAFG00000012963 | −1.04699494 | 1.30E−28 |
| CDO1 | −1.25074668 | 3.02E−17 |
| TF.1 | −1.25650717 | 1.71E−24 |
| SCRG1 | −1.30522628 | 1.67E−29 |
| C2orf40 | −1.34826821 | 2.43E−35 |
| COL2A1 | −1.59138813 | 8.90E−44 |

TABLE 3B

| Genes | avg_logFC | p_val_adj |
|---|---|---|
| SERPINE1 | 2.270134566 | 1.64E−124 |
| PLAU | 1.938321329 | 3.33E−101 |
| TF | 1.615323222 | 8.66E−70 |
| CYP1B1 | 1.605188409 | 3.59E−52 |
| PTGS2 | 1.601522321 | 2.60E−53 |
| CTGF | 1.594792721 | 2.67E−101 |
| GADD45B | 1.575014248 | 2.41E−111 |
| PMEPA1 | 1.368238395 | 1.77E−138 |
| RGS2 | 1.33586132 | 3.82E−30 |
| LIF | 1.185820503 | 2.35E−68 |
| ENSCAFG00000007045 | 1.127510697 | 4.59E−56 |
| PLAUR | 1.080355294 | 2.92E−83 |
| BHLHE40 | 0.987427044 | 2.44E−75 |
| STC2 | 0.973849543 | 1.62E−60 |
| GDNF | 0.951146608 | 1.94E−65 |
| INHBA | 0.938581446 | 1.09E−42 |
| FSTL3 | 0.915068598 | 1.25E−68 |
| ALAS1 | 0.910462856 | 9.86E−46 |
| DDX58 | 0.907727781 | 7.51E−05 |
| SNAI1 | 0.880863852 | 3.42E−59 |
| HAS2 | 0.831263218 | 1.42E−38 |
| CYR61 | 0.822041938 | 1.81E−55 |
| VCAN | 0.791744247 | 3.50E−53 |
| HBEGF | 0.743209509 | 3.29E−31 |
| LTBP1 | 0.726556801 | 6.54E−43 |
| UGDH | 0.723736501 | 2.68E−54 |
| SKIL | 0.722523271 | 1.13E−68 |
| ENSCAFG00000032813 | 0.69050212 | 2.99E−22 |
| CYP26B1 | 0.66026285 | 7.51E−06 |
| EDN1 | 0.659509753 | 1.23E−11 |
| FST | 0.649885638 | 1.53E−06 |
| RASL11A | 0.643700697 | 1 |
| PTHLH | 0.632177886 | 5.00E−13 |
| TFPI2 | 0.631465532 | 3.09E−31 |
| BAZ1A | 0.625318472 | 3.17E−59 |
| LRRC8C | 0.613489029 | 3.03E−43 |
| PDPN | 0.612788854 | 1.17E−25 |
| ISG20 | 0.610179996 | 1.71E−28 |
| ACTN1 | 0.607997286 | 7.22E−54 |
| THBS1 | 0.572688209 | 1.13E−34 |
| ENSCAFG00000015625 | 0.569353099 | 4.40E−22 |
| ENSCAFG00000002015 | 0.566931371 | 2.05E−46 |
| CYCS | 0.565808469 | 3.55E−63 |
| PDE4B | 0.564477965 | 1.51E−27 |
| COL5A1 | 0.55491057 | 6.15E−27 |
| PDLIM5 | 0.55338401 | 5.05E−42 |
| ELOVL4 | 0.541004424 | 8.53E−32 |
| ICAM1 | 0.540189379 | 1.40E−31 |
| FOXP4 | 0.538976974 | 5.14E−38 |
| NGF | 0.536034633 | 7.86E−25 |
| RBMS1 | 0.520889735 | 1.39E−56 |
| ACTA2 | 0.51835637 | 4.77E−05 |
| ENSCAFG00000033998 | 0.510076931 | 2.01E−21 |
| ADSS | 0.508717264 | 2.27E−29 |
| WISP1 | 0.505862364 | 1.47E−14 |
| ELK3 | 0.505306706 | 4.15E−28 |
| MAP1B | 0.501889012 | 3.96E−29 |
| S100A2 | 0.501136569 | 1 |
| MARCH3 | 0.495772749 | 1.36E−25 |
| NF2 | 0.494490521 | 4.59E−16 |
| ENSCAFG00000003758 | 0.49399392 | 1.14E−30 |
| SNRNP48 | 0.489452297 | 3.82E−37 |
| NIPAL1 | 0.488938551 | 9.16E−25 |
| TNFSF10 | 0.487114772 | 1.26E−27 |
| MT2A | 0.484966088 | 9.09E−27 |
| MEDAG | 0.483035018 | 5.29E−14 |
| COL1A1 | 0.481300099 | 2.93E−14 |
| IFRD1 | 0.47940483 | 1.85E−26 |
| DNAJB4 | 0.478466459 | 2.86E−25 |
| KCTD10 | 0.472668513 | 7.71E−34 |
| RCAN1 | 0.467451478 | 2.95E−19 |
| PALLD | 0.46704451 | 3.09E−25 |
| FZD2 | 0.461441264 | 3.70E−42 |
| KIF5B | 0.452934638 | 9.92E−51 |
| FN1 | 0.451706657 | 7.67E−21 |
| ENSCAFG00000006436 | 0.450184308 | 9.50E−28 |
| GLRX | 0.447673859 | 7.04E−08 |
| VPS37A | 0.44658027 | 6.47E−31 |
| SLC4A7 | 0.44656017 | 8.43E−31 |
| ENSCAFG00000037735 | 0.44491503 | 9.27E−20 |
| NEK7 | 0.440671399 | 1.16E−23 |
| ENSCAFG00000024171 | 0.439808333 | 2.72E−13 |
| PLOD2 | 0.435799011 | 5.58E−32 |
| SSX2IP | 0.433433068 | 5.40E−22 |
| HIP1 | 0.430291869 | 2.35E−28 |
| ELL2 | 0.429880706 | 4.21E−26 |
| PTK7 | 0.428408906 | 2.31E−26 |
| ANO6 | 0.42819938 | 6.54E−27 |
| ETS1 | 0.42651363 | 1.16E−23 |
| COL8A1 | 0.425783098 | 1.12E−05 |
| IQCJ-SCHIP1 | 0.421507132 | 2.17E−15 |
| LOX | 0.421443873 | 1.73E−18 |
| SH3BP5L | 0.420108354 | 8.54E−23 |
| RAI14 | 0.418838543 | 5.16E−24 |
| SNX10 | 0.416905955 | 2.20E−24 |
| ADAM19 | 0.416225598 | 7.29E−18 |
| COL12A1 | 0.415635917 | 3.05E−22 |
| HSPH1 | 0.412816956 | 6.08E−27 |
| SRPX2 | 0.412666738 | 1.53E−24 |
| DLC1 | 0.411009682 | 2.22E−19 |
| ALDH18A1 | 0.410604989 | 6.49E−33 |
| AMOTL2 | 0.410370891 | 1.12E−18 |
| SDC4 | 0.406600535 | 1.73E−09 |
| PFKFB3 | 0.403011464 | 1 |
| NRP2 | 0.402252102 | 9.73E−24 |
| ENSCAFG00000000345 | 0.398102466 | 6.59E−26 |
| CRABP2 | 0.396979592 | 2.93E−14 |
| GORAB | 0.396186256 | 1.23E−15 |
| TPM2 | 0.394423601 | 0.0010039 |
| LUM | 0.392747857 | 2.43E−05 |
| ENSCAFG00000013979 | 0.388719855 | 3.97E−14 |
| SPHK1 | 0.38692647 | 4.99E−28 |
| IFIH1 | 0.38656085 | 7.45E−06 |
| COL3A1 | 0.383267542 | 4.72E−13 |
| BGN | 0.383210386 | 5.66E−14 |
| PICALM | 0.382048887 | 1.62E−29 |
| RALA | 0.377466022 | 4.13E−22 |
| ITGA5 | 0.372426688 | 0.00022458 |
| ZNFX1 | 0.371873777 | 2.03E−23 |
| ENSCAFG00000036684 | 0.371392695 | 1.83E−17 |
| MAP7D1 | 0.367099126 | 8.37E−18 |
| SRM | 0.366802221 | 1.71E−19 |
| BCL2L11 | 0.366623544 | 1.54E−14 |
| AEN | 0.366556769 | 4.58E−09 |
| MYH9 | 0.365525825 | 1.82E−23 |
| YIF1B | 0.364924894 | 5.58E−20 |
| NFKBIA | 0.363974328 | 0.00098373 |
| PHF20L1 | 0.363486584 | 5.95E−20 |
| PDZRN3 | 0.361763681 | 3.68E−24 |
| BLOC1S2 | 0.360110531 | 8.67E−25 |
| GTF2F1 | 0.359984498 | 7.39E−18 |
| PTGES | 0.354877191 | 1 |
| SPOP | 0.354769454 | 3.71E−17 |
| BCAR1 | 0.354670991 | 5.43E−18 |
| GFPT2 | 0.353801801 | 3.84E−08 |
| CKAP4 | 0.353447818 | 6.34E−26 |
| ENSCAFG00000031852 | 0.351467487 | 9.04E−27 |
| TUBA4A | 0.351136866 | 3.09E−12 |
| PNPLA2 | 0.351032625 | 5.69E−18 |
| CTNNB1 | 0.348949126 | 7.58E−35 |
| FAS | 0.348560418 | 7.60E−17 |
| SYNC | 0.346548987 | 2.88E−07 |
| MMD | 0.345335146 | 1.13E−15 |
| TCF4 | 0.34342355 | 2.19E−13 |
| TCIRG1 | 0.341695915 | 1.25E−15 |
| IBTK | 0.341267978 | 1.95E−18 |
| GOLT1B | 0.340457605 | 3.76E−21 |
| SNRNP40 | 0.34009679 | 1.00E−17 |
| COL1A2 | 0.339411575 | 2.05E−10 |
| HAPLN3 | 0.33610249 | 5.47E−13 |
| SACS | 0.33507603 | 4.06E−13 |
| PINX1 | 0.334967829 | 2.96E−13 |
| CDK8 | 0.334822383 | 1.34E−18 |
| ENSCAFG00000011482 | 0.332844714 | 2.69E−14 |
| UBE2J1 | 0.332459 | 8.26E−16 |
| S100A4 | 0.331511753 | 4.16E−05 |
| STARD13 | 0.330863114 | 7.13E−12 |
| SRSF7 | 0.330639938 | 2.33E−16 |
| MPP6 | 0.330320089 | 1.35E−13 |
| STK38L | 0.330194066 | 1.22E−11 |
| VEGFA | 0.326552564 | 7.11E−07 |
| GNL3 | 0.326489092 | 1.78E−15 |
| COL5A2 | 0.325283785 | 1.90E−16 |
| PID1 | 0.324789537 | 5.20E−07 |
| NUAK1 | 0.322608179 | 7.38E−12 |
| LMCD1 | 0.322505239 | 0.00031673 |
| LPCAT2 | 0.321883143 | 2.06E−12 |
| THBS2 | 0.321515274 | 9.22E−06 |
| ENSCAFG00000037410 | 0.321415081 | 1.18E−16 |
| ATP13A3 | 0.320744061 | 8.61E−15 |
| ANKRD10 | 0.320687221 | 2.04E−14 |
| TMEM2 | 0.320271599 | 6.72E−11 |
| ENSCAFG00000018440 | 0.318205368 | 3.56E−12 |
| REL | 0.316388368 | 3.30E−17 |
| SNAI2 | 0.316301052 | 1 |
| ENSCAFG00000001840 | 0.315952691 | 6.09E−13 |
| TXNRD1 | 0.314990868 | 2.15E−18 |
| RIC1 | 0.31260878 | 2.06E−14 |
| SMC5 | 0.312166654 | 8.07E−13 |
| NDUFAF4 | 0.312125069 | 1.97E−13 |
| NOP58 | 0.312027948 | 1.77E−09 |
| CSRP1 | 0.311483153 | 1.84E−23 |
| TMEM41B | 0.311216589 | 2.61E−12 |
| ENSCAFG00000017326 | 0.309316441 | 0.65348525 |
| NUDC | 0.308853972 | 1.66E−19 |
| STAM2 | 0.308195198 | 4.20E−20 |
| ENSCAFG00000001448 | 0.307218421 | 4.61E−30 |
| MYLK | 0.307130976 | 2.61E−09 |
| ACVR1 | 0.30491048 | 5.65E−13 |
| RNF6 | 0.304133178 | 1.63E−16 |
| CCT4 | 0.303922731 | 2.48E−17 |
| FAM49B | 0.302795732 | 1.45E−13 |
| TKT | 0.302581621 | 8.90E−06 |
| PCSK1 | 0.302052584 | 1.88E−31 |
| SMG7 | 0.301762461 | 1.82E−12 |
| MRPS31 | 0.301222195 | 6.87E−19 |
| TUBA1C | 0.300991012 | 2.83E−10 |
| UBE2S | 0.300842341 | 1.06E−13 |
| NUDCD1 | 0.29796364 | 2.49E−14 |
| CRLF3 | 0.297570697 | 9.93E−15 |
| TWF1 | 0.296148932 | 6.20E−16 |
| DDX60 | 0.295595225 | 1 |
| GTF2H1 | 0.29497148 | 3.45E−15 |
| FAM126A | 0.294401112 | 3.95E−12 |
| PNPLA8 | 0.292016173 | 1.55E−15 |
| FAM20C | 0.291791458 | 2.00E−09 |
| MT1 | 0.290657433 | 0.14826836 |
| DNAJB9 | 0.289623435 | 2.82E−12 |
| ENSCAFG00000029851 | 0.289545577 | 3.15E−14 |
| PRPF38B | 0.288814072 | 1.03E−13 |
| RPS6KC1 | 0.288104439 | 1.19E−14 |
| DKK1 | 0.288032443 | 3.79E−07 |
| ENSCAFG00000030449 | 0.285576385 | 5.59E−07 |
| DPP4 | 0.285457297 | 2.29E−11 |
| FLNB | 0.284752033 | 1.74E−16 |
| PLS3 | 0.284373241 | 5.79E−12 |
| CLCF1 | 0.284081513 | 4.42E−24 |
| ATP6V1H | 0.280867753 | 4.10E−12 |
| GPATCH4 | 0.280788015 | 3.35E−09 |
| ENSCAFG00000007306 | 0.279060899 | 1.93E−13 |
| TMF1 | 0.278899959 | 4.01E−12 |
| MAOA | 0.277829558 | 1.24E−11 |
| NAV3 | 0.277094704 | 1.59E−10 |
| SOD2 | 0.276366887 | 1.30E−12 |
| EMD | 0.276004674 | 1.41E−10 |
| ANKRD37 | 0.275977979 | 1 |
| PLK2 | 0.274743272 | 2.22E−13 |
| NEXN | 0.27437062 | 8.54E−11 |
| SEC24D | 0.27395841 | 8.01E−12 |
| C11orf24 | 0.27329155 | 5.01E−10 |
| C10orf90 | 0.273012658 | 7.82E−14 |
| NRBF2 | 0.272928691 | 4.97E−11 |
| YRDC | 0.272465098 | 6.79E−11 |
| ADAM12 | 0.272089054 | 3.81E−11 |
| DSE | 0.271615184 | 9.19E−10 |
| GLO1 | 0.269818214 | 1.01E−13 |
| WNT9A | 0.269258272 | 5.10E−17 |
| TMEM218 | 0.268923991 | 1.14E−08 |
| BNC1 | 0.268795705 | 1.05E−22 |
| GPNMB | 0.267056623 | 0.01187383 |
| ENSCAFG00000012041 | 0.266965762 | 1.07E−10 |
| PPP1R15B | 0.266453221 | 1.78E−10 |
| SEMA3C | 0.265622693 | 5.19E−08 |
| STXBP6 | 0.265520629 | 4.60E−08 |
| PTPN12 | 0.265435261 | 4.09E−11 |
| SRSF11 | 0.265125218 | 4.34E−13 |
| MAPK8 | 0.265047739 | 8.76E−12 |
| UGCG | 0.264965223 | 4.27E−06 |
| MSN | 0.264350131 | 5.07E−14 |
| ACTG2 | 0.264339894 | 1 |
| FADS1 | 0.263944354 | 3.63E−06 |
| SLC25A16 | 0.263112844 | 3.93E−11 |
| TIMM8A | 0.262918333 | 7.11E−10 |
| ENSCAFG00000016011 | 0.262799466 | 1.59E−11 |
| SLC20A1 | 0.261386402 | 8.28E−09 |
| PHAX | 0.259855919 | 4.67E−12 |
| FAM46A | 0.259808127 | 0.00076126 |
| C1orf52 | 0.258140183 | 9.52E−09 |
| SNX16 | 0.257966718 | 5.26E−09 |
| STX7 | 0.257533159 | 6.61E−10 |
| EIF1AD | 0.257048668 | 6.20E−12 |
| ENSCAFG00000002833 | 0.256902211 | 1.84E−10 |
| LGALS3 | 0.25589799 | 2.41E−12 |
| ENSCAFG00000009110 | 0.255855497 | 0.00319632 |
| PA2G4 | 0.255149397 | 9.63E−09 |
| DNAJC25 | 0.255086843 | 2.37E−11 |
| MYBL1 | 0.255034351 | 8.97E−07 |
| TGIF2-C20orf24 | 0.254704432 | 4.74E−24 |
| MZT1 | 0.253444595 | 2.30E−19 |
| EGR1 | 0.253387664 | 0.00017755 |
| ABRACL | 0.253085413 | 2.76E−09 |
| DUS4L | 0.253085413 | 1.08E−12 |
| WDR70 | 0.253085413 | 1.20E−11 |
| ENSCAFG00000006693 | 0.252862024 | 1.44E−12 |
| ENSCAFG00000015396 | 0.252221482 | 9.63E−11 |
| MYO1E | 0.251784176 | 2.76E−08 |
| ALG5 | 0.251159176 | 6.87E−13 |
| SYTL4 | 0.250850775 | 1.78E−08 |
| HAUS3 | 0.250669369 | 7.79E−12 |
| LIN7B | 0.250504766 | 9.14E−07 |
| ENSCAFG00000019620 | 0.250464179 | 4.76E−07 |
| ND4L | −0.2502282 | 1.22E−11 |
| ENSCAFG00000006810 | −0.2504346 | 1.18E−35 |
| ENSCAFG00000001968 | −0.25169181 | 3.34E−09 |
| ENSCAFG00000029055 | −0.25234755 | 1.34E−11 |
| KIAA1217 | −0.2529506 | 0.00179526 |
| ENSCAFG00000023205 | −0.25319681 | 1.13E−09 |
| SLC38A2 | −0.25463943 | 3.95E−09 |
| RPL27A | −0.25527852 | 6.77E−19 |
| ATP5F1 | −0.25558736 | 2.46E−12 |
| SYNE2 | −0.25563906 | 0.00564005 |
| FTH1.1 | −0.25601459 | 1.65E−28 |
| IRX2 | −0.25697068 | 4.15E−07 |
| RPS23 | −0.25707733 | 3.24E−66 |
| PLEKHA2 | −0.25732945 | 4.85E−10 |
| TMSB10 | −0.25738773 | 1.06E−07 |
| EFEMP2 | −0.25774021 | 2.43E−06 |
| RPL32 | −0.2580165 | 1.35E−68 |
| RPS16 | −0.26051969 | 6.36E−53 |
| OLFM1 | −0.26165411 | 5.40E−06 |
| MATN2 | −0.26173223 | 0.22784127 |
| CA9 | −0.26259303 | 0.00077323 |
| ENSCAFG00000031486 | −0.26282112 | 3.03E−12 |
| ENSCAFG00000031458 | −0.26366542 | 6.53E−27 |
| EIF3G | −0.2640728 | 1.09E−10 |
| RPS8 | −0.26452057 | 5.76E−52 |
| ENSCAFG00000013201 | −0.26458174 | 1.98E−09 |
| NCBP2 | −0.26478778 | 1.63E−14 |
| IRF3 | −0.26479261 | 2.42E−05 |
| COL11A1 | −0.26495472 | 2.11E−07 |
| ATP5J | −0.26513943 | 7.57E−36 |
| ENSCAFG00000001214 | −0.26698805 | 4.12E−43 |
| HAGH | −0.26721896 | 4.44E−05 |
| SNRPG | −0.26737977 | 1.23E−28 |
| RPS27A | −0.267553 | 7.06E−50 |
| PLA2G7 | −0.26798435 | 8.00E−05 |
| COX7A2 | −0.26828031 | 2.01E−33 |
| CRYL1 | −0.26869155 | 0.03987256 |
| UQCC2 | −0.26872058 | 3.03E−20 |
| RPS19 | −0.26965853 | 2.54E−54 |
| LSM8 | −0.27001268 | 1.71E−17 |
| ENSCAFG00000033694 | −0.27033944 | 1.27E−05 |
| NAA38 | −0.27091997 | 1.03E−13 |
| ENSCAFG00000013214 | −0.27116323 | 0.0001272 |
| ENSCAFG00000015445 | −0.27222156 | 3.77E−14 |
| EIF3H | −0.27225303 | 5.17E−15 |
| FAM162A | −0.27255667 | 5.10E−19 |
| ENSCAFG00000031671 | −0.27326291 | 1.45E−12 |
| DHRS7 | −0.27358298 | 1.57E−13 |
| ENSCAFG00000009523 | −0.27388325 | 3.12E−65 |
| ENSCAFG00000024837 | −0.27503938 | 1.26E−06 |
| CAV1 | −0.27575872 | 1.13E−08 |
| PIK3R1 | −0.27616776 | 6.20E−08 |
| PNP | −0.27624136 | 1.22E−10 |
| RPS3 | −0.27646423 | 4.97E−41 |
| PRDX6 | −0.27721779 | 2.74E−10 |
| SYNE1 | −0.27766239 | 1.12E−06 |
| TGFB2 | −0.27860449 | 1.20E−05 |
| C19orf70 | −0.27913707 | 1.68E−16 |
| RPL9 | −0.27933313 | 3.27E−56 |
| UBA52 | −0.27967552 | 1.06E−61 |
| IRX3 | −0.27994437 | 2.12E−12 |
| ENSCAFG00000000567 | −0.28048729 | 3.78E−32 |
| SIX1 | −0.28063403 | 1.81E−08 |
| ENSCAFG00000031583 | −0.2822734 | 5.57E−05 |
| COL8A2 | −0.28259098 | 2.47E−06 |
| TMEM47 | −0.28263776 | 7.01E−06 |
| RPS14 | −0.28359277 | 3.94E−64 |
| ENSCAFG00000024413 | −0.28407068 | 1.57E−22 |
| EIF4A2 | −0.28532648 | 2.77E−12 |
| RPS15 | −0.28589156 | 1.15E−73 |
| PTRHD1 | −0.28636413 | 2.85E−09 |
| SCARB1 | −0.28673244 | 0.00013137 |
| RPL5 | −0.28692681 | 1.38E−38 |
| MRPL36 | −0.28821789 | 2.66E−19 |
| UQCRQ | −0.28822847 | 3.47E−22 |
| TMEM106C | −0.28847789 | 9.60E−10 |
| NUDT2 | −0.28939319 | 1.74E−10 |
| ENSCAFG00000000061 | −0.28999226 | 3.63E−16 |
| HP1BP3 | −0.29072458 | 3.45E−10 |
| COX4I1 | −0.29091796 | 1.88E−26 |
| MINOS1 | −0.29188549 | 7.83E−13 |
| RPS6 | −0.29213686 | 4.42E−59 |
| ENSCAFG00000007990 | −0.29262724 | 4.16E−53 |
| ENSCAFG00000014234 | −0.29369794 | 3.16E−14 |
| DDOST | −0.29460599 | 4.75E−12 |
| NDUFS5 | −0.29480992 | 3.04E−14 |
| ENSCAFG00000025332 | −0.29552836 | 3.73E−08 |
| OCIAD2 | −0.29661566 | 6.10E−05 |
| RPL23 | −0.29704381 | 4.23E−14 |
| PLCD1 | −0.29814697 | 9.30E−09 |
| ENSCAFG00000008935 | −0.29847055 | 3.16E−16 |
| RPS5 | −0.29888365 | 8.74E−49 |
| KRTCAP2 | −0.29965666 | 3.53E−32 |
| RPS7 | −0.3001245 | 3.11E−61 |
| ADGRG1 | −0.30027415 | 8.02E−07 |
| AK1 | −0.3003055 | 1.74E−17 |
| ETV5 | −0.30032907 | 0.00065529 |
| CUTA | −0.30090167 | 2.16E−15 |
| MACF1 | −0.3025817 | 2.22E−14 |
| S100A13 | −0.30389326 | 3.77E−21 |
| RPL11 | −0.3055851 | 1.77E−61 |
| KIAA1551 | −0.30634507 | 7.15E−05 |
| DDX41 | −0.30678774 | 1.74E−09 |
| ENSCAFG00000010850 | −0.30709786 | 7.99E−21 |
| ENSCAFG00000004800 | −0.30879851 | 8.64E−20 |
| RPL14 | −0.30950893 | 3.75E−53 |
| ATPIF1 | −0.31009434 | 1.05E−30 |
| ENSCAFG00000024445 | −0.31072658 | 8.93E−17 |
| ENSCAFG00000019141 | −0.31082896 | 3.03E−12 |
| PDGFRA | −0.31213428 | 3.66E−09 |
| MYLIP | −0.31239862 | 0.00305231 |
| WNK1 | −0.31371948 | 2.85E−06 |
| RPL15 | −0.31381374 | 6.96E−58 |
| CPNE2 | −0.31450523 | 8.40E−11 |
| NSMCE1 | −0.31690314 | 4.08E−15 |
| ENSCAFG00000008336 | −0.31773946 | 7.54E−15 |
| S100A6 | −0.31834723 | 7.14E−29 |
| ENSCAFG00000029635 | −0.31853919 | 2.16E−27 |
| RPL23.1 | −0.31897707 | 4.47E−75 |
| ALDOC | −0.3202288 | 1.44E−05 |
| MET | −0.32041132 | 1.58E−12 |
| RPL28 | −0.32114441 | 1.45E−72 |
| MT-ND3 | −0.32117437 | 5.90E−24 |
| sept-11 | −0.32177457 | 5.49E−06 |
| ENSCAFG00000012725 | −0.3241188 | 9.34E−84 |
| ENSCAFG00000008776 | −0.32856626 | 4.48E−24 |
| CAV2 | −0.32918041 | 6.60E−21 |
| ENSCAFG00000012395 | −0.3294887 | 3.14E−79 |
| RACK1 | −0.33114297 | 7.96E−31 |
| FAIM | −0.33213103 | 8.41E−10 |
| PERP | −0.33359759 | 1.78E−15 |
| ENSCAFG00000012553 | −0.33360054 | 5.06E−07 |
| ENSCAFG00000006545 | −0.33425112 | 8.50E−24 |
| CDON | −0.3359286 | 5.67E−05 |
| DCXR | −0.33648006 | 3.65E−14 |
| ENSCAFG00000012676 | −0.33764386 | 8.57E−50 |
| RPS9 | −0.33797823 | 1.18E−68 |
| NR4A2 | −0.33836487 | 1 |
| EGLN1 | −0.33838955 | 1.26E−14 |
| RPL7A | −0.33906574 | 7.16E−43 |
| SERTAD4 | −0.34066495 | 4.26E−08 |
| RPL31 | −0.34097289 | 9.02E−43 |
| PRDX4 | −0.34132246 | 1.23E−05 |
| LSP1 | −0.34237651 | 4.73E−06 |
| PFDN5.1 | −0.34415804 | 3.86E−40 |
| THRAP3 | −0.34500485 | 2.12E−15 |
| SPTLC3 | −0.34527319 | 1.07E−06 |
| RPL27 | −0.34716721 | 6.72E−83 |
| COX7A1 | −0.3477697 | 1.62E−12 |
| ENSCAFG00000031952 | −0.34871773 | 1.69E−75 |
| ENSCAFG00000006734 | −0.34916722 | 4.98E−47 |
| CD59 | −0.34939808 | 2.99E−08 |
| ENSCAFG00000014648 | −0.35067979 | 1.17E−31 |
| TMA7 | −0.35455243 | 6.98E−28 |
| ENSCAFG00000000377 | −0.35513972 | 3.26E−65 |
| ENSCAFG00000008873 | −0.35553678 | 3.16E−29 |
| SSBP2 | −0.35603916 | 1.06E−16 |
| TSTD1 | −0.35753604 | 3.81E−20 |
| NAP1L1 | −0.35787215 | 2.53E−31 |
| ENSCAFG00000007199 | −0.35808819 | 1.69E−07 |
| IFI30 | −0.35851738 | 1.95E−16 |
| RPS18 | −0.35930671 | 1.63E−66 |
| ITM2C | −0.35992537 | 1.12E−11 |
| GLI3 | −0.36064372 | 9.71E−16 |
| RPS4X | −0.36073595 | 7.39E−68 |
| RPS17 | −0.36250787 | 2.54E−86 |
| MALL | −0.36321906 | 0.00544896 |
| NDUFB2 | −0.363249 | 1.47E−47 |
| ENSCAFG00000012839 | −0.36479984 | 2.36E−28 |
| ANLN | −0.36493066 | 4.03E−08 |
| NPNT | −0.3652741 | 0.00040752 |
| ENSCAFG00000033303 | −0.36765835 | 1.85E−11 |
| PTMA | −0.37007399 | 9.31E−13 |
| SSR4 | −0.37115863 | 1.71E−49 |
| ENSCAFG00000018187 | −0.37257065 | 9.37E−30 |
| CDKN2C | −0.37273569 | 3.27E−14 |
| ARID5B | −0.37401241 | 3.20E−10 |
| ENSCAFG00000032537 | −0.37489109 | 2.01E−29 |
| FTL | −0.37512085 | 6.70E−37 |
| RPL10A | −0.37521784 | 5.96E−69 |
| timp3 | −0.3754959 | 1.45E−06 |
| EFEMP1 | −0.37717726 | 1.66E−05 |
| BNIP3 | −0.37754141 | 3.43E−18 |
| NFIB | −0.37790027 | 3.52E−16 |
| ENSCAFG00000004260 | −0.38047277 | 4.07E−42 |
| ENSCAFG00000030466 | −0.38162162 | 1.15E−20 |
| KLF5 | −0.38266125 | 5.01E−10 |
| ENSCAFG00000015433 | −0.38290335 | 3.48E−35 |
| TRPS1 | −0.38607454 | 9.04E−23 |
| GLTSCR2 | −0.38646882 | 8.82E−17 |
| LRIG1 | −0.38741804 | 0.45650294 |
| RPL35 | −0.38837899 | 6.40E−96 |
| EPS8 | −0.38939014 | 1.05E−15 |
| ENSCAFG00000018784 | −0.39083723 | 3.47E−79 |
| RPS15A | −0.39507679 | 1.47E−95 |
| ENSCAFG00000016065 | −0.39527975 | 4.76E−29 |
| EIF3E | −0.39793997 | 4.14E−30 |
| SOX9 | −0.39832216 | 2.28E−15 |
| HSP90AB1 | −0.39919468 | 1.14E−23 |
| RPL7.1 | −0.40252969 | 9.64E−57 |
| USP6NL | −0.4044903 | 9.81E−09 |
| KARS | −0.40685681 | 4.09E−29 |
| ENSCAFG00000032152 | −0.40705689 | 2.87E−50 |
| FOS | −0.40760097 | 6.05E−12 |
| EIF3K | −0.40791741 | 1.51E−55 |
| ENSCAFG00000031706 | −0.40811952 | 5.87E−14 |
| HMGB2 | −0.41088923 | 3.15E−13 |
| CCND1 | −0.41110376 | 3.79E−12 |
| GM2A | −0.41336979 | 2.29E−12 |
| AIF1L | −0.41497493 | 1.01E−12 |
| ENSCAFG00000028936 | −0.41544652 | 1.22E−15 |
| RPS27 | −0.41766771 | 2.25E−84 |
| RDH10 | −0.41857351 | 1 |
| DBI | −0.42212407 | 1.39E−26 |
| EEF1D | −0.42420928 | 1.23E−48 |
| ENSCAFG00000031955 | −0.42524668 | 3.47E−101 |
| LIMCH1 | −0.42552716 | 1.02E−12 |
| ENSCAFG00000014958 | −0.42561197 | 1.06E−84 |
| PPP1R1B | −0.43481929 | 9.79E−16 |
| ENSCAFG00000029945 | −0.4358663 | 1.27E−51 |
| ABLIM1 | −0.43808576 | 4.33E−09 |
| MEI4 | −0.43823361 | 6.55E−65 |
| CST6 | −0.43891285 | 6.44E−21 |
| ENSCAFG00000028960 | −0.4393504 | 2.84E−44 |
| ENSCAFG00000012624 | −0.43958546 | 1.07E−36 |
| PPP1R3C | −0.4414792 | 2.49E−13 |
| ZFHX3 | −0.44526818 | 4.05E−21 |
| FRMD6 | −0.44801131 | 5.66E−19 |
| RPL3 | −0.44877661 | 3.98E−61 |
| ENSCAFG00000003564 | −0.4536463 | 1.24E−44 |
| DEK | −0.45464618 | 3.20E−21 |
| ENSCAFG00000032920 | −0.45475746 | 2.07E−27 |
| ZBTB16 | −0.45556596 | 6.23E−25 |
| ENSCAFG00000019044 | −0.46149169 | 4.79E−97 |
| INSIG1 | −0.46389424 | 2.41E−15 |
| FBXO2 | −0.46751861 | 2.22E−13 |
| IL18BP | −0.4702197 | 0.00212428 |
| EIF3L | −0.47603295 | 4.38E−32 |
| TFRC | −0.48601212 | 2.49E−21 |
| TSPAN13 | −0.48711431 | 7.58E−11 |
| PTGDR | −0.49035848 | 0.00147465 |
| BGLAP | −0.4908875 | 0.00032211 |
| GOLIM4 | −0.49288747 | 9.91E−13 |
| NUDT4 | −0.49350073 | 4.93E−15 |
| ENSCAFG00000032717 | −0.49606124 | 4.74E−93 |
| ZNF395 | −0.50102873 | 5.16E−29 |
| ENSCAFG00000024643 | −0.50180857 | 1.14E−49 |
| TSC22D3 | −0.50190377 | 2.67E−22 |
| CRYAB | −0.5056394 | 1.42E−14 |
| BBX | −0.50647686 | 6.73E−27 |
| ENSCAFG00000015834 | −0.51931737 | 3.02E−77 |
| ENSCAFG00000015372 | −0.51998521 | 3.15E−22 |
| BMP4 | −0.52039254 | 1 |
| SLC1A1 | −0.52843445 | 1.88E−10 |
| CADM1 | −0.5363386 | 3.68E−16 |
| ENSCAFG00000000027 | −0.5368636 | 3.09E−68 |
| AHNAK | −0.53702784 | 1.49E−15 |
| ENSCAFG00000017688 | −0.53835656 | 1.13E−100 |
| IMPDH2 | −0.5387333 | 2.55E−32 |
| FDFT1 | −0.55181825 | 3.95E−23 |
| TFAP2C | −0.55331187 | 8.72E−22 |
| ENSCAFG00000006884 | −0.55630239 | 6.04E−58 |
| APOE | −0.55635733 | 0.00074245 |
| MEF2C | −0.55718713 | 1.64E−21 |
| GSTM3 | −0.5694882 | 5.24E−14 |
| OGFRL1 | −0.57110992 | 4.48E−15 |
| TM4SF18 | −0.59863723 | 5.61E−19 |
| PRELP | −0.61360364 | 1.32E−22 |
| TNNI2 | −0.61963121 | 2.31E−18 |
| TGFBR3 | −0.62222398 | 4.53E−21 |
| IL33 | −0.627616 | 1.88E−13 |
| ABI3BP | −0.63172183 | 1.59E−14 |
| CLU | −0.63292644 | 1.21E−13 |
| SCG5 | −0.63496211 | 9.07E−22 |
| NDP | −0.64329834 | 4.58E−20 |
| ENSCAFG00000030307 | −0.64707163 | 7.11E−17 |
| ENSCAFG00000029553 | −0.66078747 | 1 |
| CTSD | −0.66916005 | 8.47E−14 |
| EIF4B | −0.68413781 | 7.28E−63 |
| EEF1A1 | −0.69294622 | 3.17E−104 |
| EDIL3 | −0.69733609 | 1.75E−28 |
| MGST1 | −0.72985887 | 3.99E−21 |
| B2M | −0.74207749 | 8.44E−26 |
| SHISA2 | −0.74376072 | 1.29E−24 |
| FGF7 | −0.74634343 | 3.26E−20 |
| FAM213B | −0.7710177 | 1.28E−22 |
| ENSCAFG00000018746 | −0.79340807 | 3.79E−22 |
| TCF7L2 | −0.85381978 | 1.01E−74 |
| ACAN | −0.87659982 | 9.99E−09 |
| ENSCAFG00000028765 | −0.93655397 | 3.09E−38 |
| MAL | −0.95458723 | 2.85E−22 |
| HAPLN1 | −0.98651343 | 1.02E−21 |
| FXYD6 | −0.98930748 | 2.20E−27 |
| FAM180A | −0.99904243 | 2.67E−19 |
| IFI6 | −1.00174878 | 6.45E−06 |
| ENSCAFG00000012963 | −1.04699494 | 1.30E−28 |
| CDO1 | −1.25074668 | 3.02E−17 |
| TF.1 | −1.25650717 | 1.71E−24 |
| SCRG1 | −1.30522628 | 1.67E−29 |
| C2orf40 | −1.34826821 | 2.43E−35 |
| COL2A1 | −1.59138813 | 8.90E−44 |

FIG. 10D shows that 200 genes were commonly-regulated in bystander versus naïve and infected versus bystander and an inverse regulation of these commonly-regulated genes was observed (FIG. 10D). IPA analysis of the differentially expressed genes provided information on the upstream regulators describing the differences between bystander and naïve cells and between infected and bystander cells. FIG. 10E shows that activation of the pathways regulated by, for example, TGF-β1, TNF, IL1β or IFN-γ can be observed when bystander cells are compared to naïve cells. This pattern is likely to reflect the reaction of bystander cells to the presence of the virus in the culture medium and to the secretion of various cytokines by cells infected with VV. By contrast, these pathways were inhibited when the IPA analysis was performed on the differentially expressed genes between infected cells and bystander cells (FIG. 10E). A similar phenomenon was observed in the second experiment (FIGS. 11D and 11E). However, in this second experiment, the number of genes modulated in bystander minus naïve cells was lower than that observed in experiment 1 (41 genes, see FIG. 11D). As the TNBC cells used in experiment 2 are 10 times less sensitive to the virus than those used in experiment 1, these differences may be attributed to a blunted ability of cells more resistant to the virus to respond to the presence of the virus in the culture medium and to stimuli secreted by infected cells. Finally, the striking contrasts between bystander minus naïve cells and infected versus bystander cells were also observed at the level of individual pathways. For example, more than 90% of genes of the IFNγ pathway that were regulated in a particular direction in bystander versus naïve cells were regulated in the opposite direction in the infected versus bystander cells (FIGS. 10F and 11F).

Identification of Genes Overrepresented in Bystander Versus Infected Cells

Inventors hypothesized that genes with “antiviral” activities were overrepresented in the bystander compared to the infected population of cells. FIG. 12A shows the Venn diagram of the genes differentially expressed in bystander cells in experiments 1 and 2. Inventors hypothesized that the 130 genes commonly regulated are candidate genes with antiviral activities (complete list in Table 4).

TABLE 4

| | avg logFC | p_val_adj | avg logFC | p_val_adj |
|---|---|---|---|---|
| Gene | Exp1 | Exp1 | Exp2 | Exp2 |
| ACLY | −0.329283973 | 6.54E−11 | −0.349044661 | 6.54E−24 |
| ACTB | −0.469139205 | 3.95E−45 | −0.334626794 | 1.73E−28 |
| ACTN1 | −0.475298081 | 6.65E−31 | −0.558511838 | 9E−68 |
| ADAM19 | −0.424573293 | 4.99E−10 | −0.415183498 | 2.65E−09 |
| ADRM1 | −0.456186589 | 9.27E−27 | −0.303533263 | 1.41E−18 |
| AKT1S1 | −0.320989682 | 4.40E−15 | −0.304187369 | 4.3E−19 |
| ALAS1 | −0.552636855 | 1.29E−11 | −0.27685229 | 0.00000189 |
| AMOTL2 | −0.764686662 | 4.89E−48 | −0.812130013 | 2.66E−55 |
| APEX1.1 | −0.469479742 | 3.92E−28 | −0.404987356 | 2.23E−28 |
| BGN | −0.298509347 | 1.17E−06 | −0.316515891 | 0.00000221 |
| BHLHE40 | −1.071225337 | 2.75E−73 | −0.895480406 | 3.12E−85 |
| BRD2 | −0.282715279 | 9.31E−12 | −0.34622725 | 6.36E−25 |
| C10orf90 | −0.379741549 | 4.16E−26 | −0.277591219 | 1.66E−13 |
| C1orf52 | −0.343773411 | 6.00E−14 | −0.282481524 | 1.3E−14 |
| CALD1 | −0.334641109 | 3.22E−15 | −0.293599277 | 0.000000036 |
| CCT3 | −0.376824068 | 4.26E−19 | −0.28290997 | 4.58E−10 |
| CKAP4 | −0.313271736 | 2.40E−18 | −0.332714027 | 9.82E−21 |
| CKB | −0.617274562 | 4.26E−33 | −0.266882954 | 0.000000522 |
| COL1A1 | −1.162298469 | 3.67E−20 | −0.730383842 | 0.0000908 |
| COL1A2 | −0.786758271 | 1.01E−33 | −0.844786243 | 0.000000683 |
| COL5A1 | −0.524523285 | 8.65E−18 | −0.678213126 | 0.0000006 |
| COL5A2 | −0.447682556 | 1.09E−24 | −0.560273726 | 6.61E−19 |
| COLGALT1 | −0.270373602 | 2.08E−07 | −0.26552084 | 0.00000661 |
| CORO1C | −0.267870464 | 4.52E−09 | −0.260000874 | 1.04E−09 |
| CSRP1 | −0.779237282 | 9.49E−66 | −0.522105366 | 3.18E−52 |
| CYP1B1 | −0.904765332 | 2.51E−10 | −0.746987036 | 1.4E−10 |
| DAPK3 | −0.403589528 | 1.52E−23 | −0.340236218 | 1.11E−17 |
| DBI | 0.355408986 | 2.40E−17 | 0.263517103 | 0.0000981 |
| DDIT4 | −1.510542809 | 8.38E−55 | −0.838454489 | 9.5E−36 |
| DPF2 | −0.269493196 | 1.29E−10 | −0.300357797 | 5.31E−18 |
| DPP4 | −0.541506 | 1.02E−23 | −0.415938661 | 0.00000304 |
| DPYSL3 | −0.327375862 | 7.72E−10 | −0.263048798 | 0.008415926 |
| DUSP1 | −0.505650506 | 1.08E−07 | −0.773988611 | 1.62E−35 |
| EDN1 | −0.680654146 | 4.15E−08 | −0.715932743 | 0.005863078 |
| EMP3 | −0.427272057 | 3.55E−21 | −0.351075708 | 0.000474705 |
| ENSCAFG00000000345 | −0.350995684 | 5.34E−16 | −0.295767863 | 7.63E−13 |
| ENSCAFG00000000657 | −0.306983874 | 7.30E−16 | −0.474942459 | 1.94E−37 |
| ENSCAFG00000004420 | −0.424823727 | 6.85E−26 | −0.455149371 | 4.67E−45 |
| ENSCAFG00000006436 | −0.280816301 | 9.86E−08 | −0.370744122 | 4.81E−25 |
| ENSCAFG00000008938 | −0.293888863 | 7.37E−13 | −0.449918727 | 3.23E−32 |
| ENSCAFG00000010572 | −0.465263129 | 1.88E−27 | −0.434060775 | 1.62E−22 |
| ENSCAFG00000014776 | −0.490637059 | 5.97E−36 | −0.391712526 | 3.12E−25 |
| ENSCAFG00000018018 | −0.390382608 | 1.40E−26 | −0.265489715 | 1.71E−17 |
| ENSCAFG00000018572 | −0.294988352 | 3.06E−09 | −0.262049056 | 0.0000023 |
| ENSCAFG00000031808 | −0.488653739 | 1.08E−20 | −0.528014059 | 1.45E−47 |
| ENSCAFG00000032813 | −1.044483723 | 9.91E−52 | −1.341946591 | 9.31E−130 |
| ENSCAFG00000034881 | −0.390719828 | 9.81E−15 | −0.413048589 | 6.16E−23 |
| ENSCAFG00000036997 | 0.486268497 | 1.54E−22 | 0.441797828 | 7.25E−31 |
| ERAL1 | −0.387475103 | 4.27E−25 | −0.421435458 | 3.1E−29 |
| FADS1 | −0.383914049 | 4.02E−10 | −0.357537112 | 1.25E−13 |
| FAM189B | −0.337542766 | 2.20E−14 | −0.309249841 | 5.56E−15 |
| FAM214B | −0.312326148 | 2.95E−14 | −0.296799665 | 7.53E−17 |
| FN1 | −0.608543099 | 1.11E−26 | −0.406097085 | 2.89E−26 |
| FOXP4 | −0.536398815 | 1.13E−32 | −0.456108271 | 5.03E−36 |
| FSTL1 | −0.279190516 | 5.50E−11 | −0.450216294 | 4.05E−13 |
| GADD45B | −0.884707566 | 9.68E−48 | −0.432057089 | 1.68E−11 |
| GATSL3 | −0.261273265 | 4.05E−12 | −0.293555976 | 1.46E−14 |
| GMPPA | −0.39945884 | 1.68E−21 | −0.338021563 | 3.9E−19 |
| GOLPH3L | 0.296079957 | 5.14E−08 | 0.362089602 | 1.09E−14 |
| HAS2 | −0.834312812 | 2.55E−24 | −1.433672384 | 7.2E−112 |
| HDGF | −0.329164669 | 1.39E−13 | −0.277244861 | 1.55E−10 |
| HINT2 | −0.350314359 | 3.38E−25 | −0.276892034 | 2.35E−14 |
| HIST1H1C | 0.507981285 | 5.45E−18 | 0.972566592 | 3.39E−34 |
| HSPBP1 | −0.302707765 | 2.61E−14 | −0.3149377 | 2.6E−18 |
| ICAM1 | −0.278006195 | 0.000285402 | −0.308369917 | 0.000203607 |
| IFRD1 | −0.506876782 | 1.97E−29 | −0.354764896 | 1.61E−14 |
| ILK | −0.663746909 | 2.23E−45 | −0.329545274 | 2.49E−17 |
| IMPDH1 | −0.359546399 | 2.29E−19 | −0.305842483 | 1.72E−17 |
| INHBA | −0.705314822 | 2.10E−17 | −0.673841379 | 8.23E−21 |
| INSIG1 | −0.517883613 | 1.55E−09 | −0.722616673 | 1.39E−20 |
| ITGA5 | −0.630697646 | 2.05E−14 | −0.645927317 | 2.83E−21 |
| JOSD2 | −0.511813789 | 5.92E−39 | −0.254008326 | 4.52E−12 |
| KCTD10 | −0.72937228 | 1.66E−56 | −0.572836982 | 9.55E−56 |
| LMNA | −0.679396038 | 4.23E−72 | −0.537495232 | 8.19E−43 |
| LTBP1 | −0.413303855 | 1.47E−13 | −0.261726869 | 0.000000614 |
| LYPLA2 | −0.27698132 | 8.23E−12 | −0.25516788 | 6.56E−12 |
| MT2A | −0.921353983 | 1.43E−48 | −0.842733001 | 2.65E−22 |
| MYC | −0.346997001 | 1.78E−07 | −0.537320871 | 2.49E−25 |
| MYH9 | −0.307629492 | 1.25E−12 | −0.39089618 | 1.59E−31 |
| MYO1C | −0.308122752 | 2.61E−18 | −0.391653281 | 5.56E−29 |
| NEDD9 | −0.453081582 | 5.38E−16 | −0.796264239 | 1.95E−35 |
| NFIC | −0.586163298 | 3.46E−53 | −0.321884893 | 2.83E−18 |
| NFKBIB | −0.258151597 | 7.58E−11 | −0.477573113 | 1.43E−41 |
| NIPAL1 | −0.343597196 | 2.60E−08 | −0.348611151 | 6.51E−11 |
| NRP2 | −0.302485917 | 4.63E−11 | −0.328120944 | 1.15E−10 |
| NUAK1 | −0.270995601 | 3.22E−06 | −0.803549652 | 1.8E−67 |
| PALLD | −0.307239937 | 3.94E−07 | −0.560157392 | 1.09E−34 |
| PDE4B | −0.748363075 | 4.21E−46 | −0.298794587 | 7.76E−12 |
| PDLIM4 | −0.314448742 | 2.73E−10 | −0.470581504 | 1.59E−09 |
| PES1 | −0.250199898 | 9.28E−12 | −0.253540363 | 2.39E−10 |
| PLAU | −0.9881659 | 1.23E−23 | −0.694747708 | 2.44E−27 |
| PLAUR | −0.386065892 | 8.95E−05 | −0.592922585 | 0.000114806 |
| PPP4R2 | 0.455421144 | 4.80E−26 | 0.250790147 | 1.55E−09 |
| PRKCDBP | −0.391057919 | 2.62E−22 | −0.29021457 | 3.04E−13 |
| PTGS2 | −1.212671535 | 3.50E−13 | −0.648757545 | 0.000918607 |
| PTRF | −0.778508357 | 1.51E−70 | −0.785306098 | 6.15E−84 |
| RAB34 | −0.30706712 | 6.25E−12 | −0.319267881 | 2.35E−18 |
| RABL6 | −0.525211976 | 1.54E−41 | −0.271450599 | 1.55E−14 |
| RGS2 | −1.251574106 | 2.85E−16 | −0.476503991 | 0.009217172 |
| RND3 | −0.363152008 | 0.000152199 | −0.270194626 | 0.00000278 |
| S100A16 | −0.465598968 | 6.51E−44 | −0.269932372 | 3.87E−20 |
| SERPINE1 | −1.544224686 | 5.20E−78 | −1.000340834 | 4.07E−47 |
| SF3A1 | −0.301170053 | 2.70E−15 | −0.350212919 | 5.42E−26 |
| SH3BP5L | −0.479466251 | 3.89E−25 | −0.269368566 | 1.45E−11 |
| SLC20A1 | −0.415955953 | 6.88E−09 | −0.783358975 | 1.75E−82 |
| SNAI1 | −0.641638892 | 4.27E−19 | −0.256041116 | 0.00000702 |
| SNAI2 | −0.50386519 | 5.92E−06 | −0.84814619 | 5.27E−30 |
| SRF | −0.280446545 | 2.38E−12 | −0.309515459 | 9.19E−16 |
| SRM | −0.388818452 | 1.20E−18 | −0.303464054 | 1.17E−09 |
| STARD13 | −0.336034522 | 5.63E−11 | −0.330861511 | 1.64E−15 |
| STUB1 | −0.273848728 | 1.05E−12 | −0.257671588 | 6.29E−12 |
| TBCB | −0.477655008 | 1.93E−30 | −0.379343953 | 4.24E−25 |
| TCF25 | −0.331267852 | 3.12E−18 | −0.283428241 | 4.49E−13 |
| TCF7L1 | −0.273800967 | 3.29E−11 | −0.316800734 | 6.6E−17 |
| TGFBR2 | −0.269221144 | 7.40E−08 | −0.337668132 | 2.41E−13 |
| THBS1 | −1.084411683 | 3.01E−71 | −0.620253313 | 1.32E−17 |
| THOC7 | −0.314026235 | 2.53E−18 | −0.259126808 | 1.25E−13 |
| TIMM8B | −0.257517912 | 1.43E−09 | −0.253825188 | 1.46E−13 |
| TKT | −0.470141327 | 1.50E−22 | −0.298197852 | 8.36E−11 |
| TMEM183A | −0.2562157 | 6.19E−12 | −0.34257659 | 2.44E−24 |
| TPI1 | −0.577773845 | 3.45E−52 | −0.364176468 | 2.86E−21 |
| TRIB2 | −0.312242832 | 4.15E−09 | −0.41557188 | 1.22E−21 |
| TSC22D3 | −0.493450123 | 1.40E−16 | −0.564867405 | 5.7E−21 |
| VCAN | −0.585749392 | 5.22E−25 | −0.26706463 | 0.006255743 |
| VEGFA | −0.413959092 | 1.57E−08 | −0.38280598 | 2.44E−08 |
| WDR1 | −0.288798004 | 2.81E−16 | −0.301814394 | 9.11E−19 |
| WDR83OS | −0.443048963 | 3.78E−35 | −0.374351384 | 6.7E−35 |
| ZFP36 | −0.451997749 | 1.02E−09 | −0.294080172 | 0.000102824 |
| ZNF703 | −0.290867823 | 2.27E−10 | −0.349563356 | 5.4E−20 |
| ZYX | −0.764047632 | 9.42E−58 | −0.689141225 | 2.07E−76 |

IPA analysis showed that these genes were consistent with an activation of the pathways regulated by TGFβ1, LPS, TNF, CTNNB1 and IL1β (FIG. 12A), suggesting that activation of these pathways are associated with an antiviral action. The comparison of the 130 candidates with genes identified as potential anti-viral genes in high throughput RNAi screens showed that only one gene, SERPINE1 was found in common with the studies of Beard et al. and none with the study of Sivan et al. (FIG. 12B).

DDIT4 Exerts an Antiviral Activity

An alternative way to analyze the dataset is to consider each individual cluster in each experiment. This analysis grants less weight to clusters with high number of cells. Inventors used the FindConservedMarkers command in Seurat v3, to run differential expression tests cluster by cluster in order to identify the conserved markers between bystander and infected cells. Inventors required a gene to have a log 2 (Fold Change)>0.25, and a maximum Bonferroni-corrected P value threshold <0.05 to be considered as a conserved marker. This analysis identifies genes that are differentially regulated between two conditions (i.e. bystander versus infected) across all clusters in one experiment. They identified 19 and 79 conserved genes in experiments 1 and 2, respectively. Interestingly, only 7 genes were conserved between the two experiments (FIG. 13A). Two of these genes were canine genes (ENSCAFG00000032813 and ENSCAF00000031808). The five remaining genes are APEX1, DDIT4, DUSP6, TBCB and DUSP1. Inventors examined the effect of DDIT4 on VV replication. Infection of HeLa cells overexpressing DDIT4 resulted in a 60% reduction in the production of infectious VV particles compared to control HeLa cells expressing GFP (FIG. 13B). Inversely, infection of mouse embryonic fibroblast (MEF) from DDIT4 knock out mice resulted in a six-fold increase in the production of infectious VV particles compared to MEF isolated from wild-type mice (FIG. 13C).

### REFERENCES

Achard, C., Surendran, A., Wedge, M. E., Ungerechts, G., Bell, J., and Ilkow, C. S. (2018). Lighting a Fire in the Tumor Microenvironment Using Oncolytic Immunotherapy. EBioMedicine 31, 17-24.
Beard, P. M., Griffiths, S. J., Gonzalez, O., Haga, L R., Pechenick Jowers, T., Reynolds, D. K., Wildenhain, J., Tekotte, H., Auer, M., Tyers, M., et al. (2014). A loss of function analysis of host factors influencing Vaccinia virus replication by RNA interference. PLoS One 9, e98431.
Ben Sahra, I., Regazzetti, C., Robert, G., Laurent, K., Le Marchand-Brustel, Y., Auberger, P., Tanti, J. F., Giorgetti-Peraldi, S., and Bost, F. (2011). Metformin, independent of AMPK, induces mTOR inhibition and cell-cycle arrest through REDD1. Cancer Res 71, 4366-4372.
Breitbach, C. J., Arulanandam, R., De Silva, N., Thorne, S. H., Patt, R., Daneshmand, M., Moon, A., Ilkow, C., Burke, J., Hwang, T. H., et al. (2013). Oncolytic vaccinia virus disrupts tumor-associated vasculature in humans. Cancer Res 73, 1265-1275.
Breitbach, C. J., Parato, K., Burke, J., Hwang, T. H., Bell, J. C., and Kirn, D. H. (2015). Pexa-Vec double agent engineered vaccinia: oncolytic and active immunotherapeutic. Curr Opin Virol 13, 49-54.
Brugarolas, J., Lei, K., Hurley, R. L., Manning, B. D., Reiling, J. H., Hafen, E., Witters, L. A., Ellisen, L. W., and Kaelin, W. G., Jr. (2004). Regulation of mTOR function in response to hypoxia by REDD1 and the TSC1/TSC2 tumor suppressor complex. Genes Dev 18, 2893-2904.
Caceres, A., Perdiguero, B., Gomez, C. E., Cepeda, M. V., Caelles, C., Sorzano, C. O., and Esteban, M. (2013). Involvement of the cellular phosphatase DUSP1 in vaccinia virus infection. PLoS Pathog 9, e1003719.
Cairns, J. (1960). The initiation of vaccinia infection. Virology 11, 603-623.
Dimier, J., Ferrier-Rembert, A., Pradeau-Aubreton, K., Hebben, M., Spehner, D., Favier, A. L., Gratier, D., Garin, D., Crance, J. M., and Drillien, R. (2011). Deletion of major nonessential genomic regions in the vaccinia virus Lister strain enhances attenuation without altering vaccine efficacy in mice. J Virol 85, 5016-5026.
Cree I A, Glaysher S, Harvey A L. (2010 August). Efficacy of anti-cancer agents in cell lines versus human primary tumour tissue. Curr Opin Pharmacol; 10(4):375-9.
Drillien, R., Spehner, D., and Hanau, D. (2004). Modified vaccinia virus Ankara induces moderate activation of human dendritic cells. J Gen Virol 85, 2167-2175.
Ellisen L W, Ramsayer K D, Johannessen C M, Yang A, Beppu H, Minda K, et al. REDD1, a developmentally regulated transcriptional target of p63 and p53, links p63 to regulation of reactive oxygen species. Mol Cell. 2002; 10(5):995-1005. Epub 2002/11/28. doi: S1097-2765(02)00706-2 [pii]. PubMed PMID: 12453409.62.
Gama, A., Alves, A., and Schmitt, F. (2008). Identification of molecular phenotypes in canine mammary carcinomas with clinical implications: application of the human classification. Virchows Arch 453, 123-132.
Gholami, S., Chen, C. H., Lou, E., De Brot, M., Fujisawa, S., Chen, N. G., Szalay, A. A., and Fong, Y. (2012). Vaccinia virus GLV-1 h153 is effective in treating and preventing metastatic triple-negative breast cancer. Ann Surg 256, 437-445.
Gillet, J. P., Varma, S., and Gottesman, M. M. (2013). The clinical relevance of cancer cell lines. J Natl Cancer Inst 105, 452-458.
Guerra, S., Caceres, A., Knobeloch, K. P., Horak, I., and Esteban, M. (2008). Vaccinia virus E3 protein prevents the antiviral action of ISG15. PLoS Pathog 4, e1000096.
Heise, C., and Kirn, D. H. (2000). Replication-selective adenoviruses as oncolytic agents. J Clin Invest 105, 847-851.
Hebben M, Brants J, Birck C, Samama J P, Wasylyk B, Spehner D, et al. High level protein expression in mammalian cells using a safe viral vector: modified vaccinia virus Ankara. Protein Expr Purif. 2007; 56(2):269-78. Epub 2007/09/26. doi: S1046-5928(07)00194-5 [pii] 10.1016/j.pep.2007.08.003. PubMed PMID: 17892951.63.
Ibrahim, N., Wicklund, A., and Wiebe, M. S. (2011). Molecular characterization of the host defense activity of the barrier to autointegration factor against vaccinia virus. J Virol 85, 11588-11600.
Jaillardon, L., Abadie, J., Godard, T., Campone, M., Loussouarn, D., Siliart, B., and Nguyen, F. (2015). The dog as a naturally-occurring model for insulin-like growth factor type 1 receptor-overexpressing breast cancer: an observational cohort study. BMC Cancer 15, 664.
Kapp, L. D., and Lorsch, J. R. (2004). The molecular mechanics of eukaryotic translation. Annu Rev Biochem 73, 657-704.
Katsafanas, G. C., and Moss, B. (2007). Colocalization of transcription and translation within cytoplasmic poxvirus factories coordinates viral expression and subjugates host functions. Cell Host Microbe 2, 221-228.
Khanna, C. (2017). Leveraging Comparative Oncology in the Hopes of Improving Therapies for Breast Cancer. Semin Oncol 44, 301.
Kim, N. H., Lim, H. Y., Im, K. S., Kim, J. H., and Sur, J. H. (2013). Identification of triple-negative and basal-like canine mammary carcinomas using four basal markers. J Comp Pathol 148, 298-306.
Kirn, D. H., and Thorne, S. H. (2009). Targeted and armed oncolytic poxviruses: a novel multi-mechanistic therapeutic class for cancer. Nat Rev Cancer 9, 64-71.
LeBlanc, A. K., Mazcko, C. N., and Khanna, C. (2016). Defining the Value of a Comparative Approach to Cancer Drug Development. Clin Cancer Res 22, 2133-2138.
Liu, D., Xiong, H., Ellis, A. E., Northrup, N. C., Rodriguez, C. O., Jr., O'Regan, R. M., Dalton, S., and Zhao, S. (2014). Molecular homology and difference between spontaneous canine mammary cancer and human breast cancer. Cancer Res 74, 5045-5056.
Martinico, S. C., Jezzard, S., Sturt, N. J., Michils, G., Tejpar, S., Phillips, R. K., and Vassaux, G. (2006). Assessment of endostatin gene therapy for familial adenomatous polyposis-related desmoid tumors. Cancer Res 66, 8233-8240.
Mercer, J., Snijder, B., Sacher, R., Burkard, C., Bleck, C. K., Stahlberg, H., Pelkmans, L., and Helenius, A. (2012). RNAi screening reveals proteasome- and Cullin3-dependent stages in vaccinia virus infection. Cell Rep 2, 1036-1047.
Montiel-Equihua C A, Martin-Duque P, de la Vieja A, Quintanilla M, Burnet J, Vassaux G, et al. Targeting sodium/iodide symporter gene expression for estrogen-regulated imaging and therapy in breast cancer. Cancer Gene Ther. 2008; 15(7):465-73. Epub 2008/04/19. doi: cgt20086 [pii]10.1038/cgt.2008.6. PubMed PMID: 18421306.59.
Paoloni, M., and Khanna, C. (2008). Translation of new cancer treatments from pet dogs to humans. Nat Rev Cancer 8, 147-156.
Pinho, S. S., Carvalho, S., Cabral, J., Reis, C. A., and Gartner, F. (2012). Canine tumors: a spontaneous animal model of human carcinogenesis. Transl Res 159, 165-172.
Pinto, J. A., Araujo, J., Cardenas, N. K., Morante, Z., Doimi, F., Vidaurre, T., Balko, J. M., and Gomez, H. L. (2016). A prognostic signature based on three-genes expression in triple-negative breast tumours with residual disease. NPJ Genom Med 1, 15015.
Pinto, J. A., Rolfo, C., Raez, L. E., Prado, A., Araujo, J. M., Bravo, L., Fajardo, W., Morante, Z. D., Aguilar, A., Neciosup, S. P., et al. (2017). In silico evaluation of DNA Damage Inducible Transcript 4 gene (DDIT4) as prognostic biomarker in several malignancies. Sci Rep 7, 1526.
Queiroga, F. L., Raposo, T., Carvalho, M. I., Prada, J., and Pires, I. (2011). Canine mammary tumours as a model to study human breast cancer: most recent findings. In Vivo 25, 455-465.
Riesco-Eizaguirre G, De la Vieja A, Rodriguez I, Miranda S, Martin-Duque P, Vassaux G, et al. Telomerase-driven expression of the sodium iodide symporter (NIS) for in vivo radioiodide treatment of cancer: a new broad-spectrum NIS-mediated antitumor approach. J Clin Endocrinol Metab. 2011; 96(9):E1435-43. Epub 2011/06/24. doi: 10.1210/jc.2010-2373 jc.2010-2373 [pii]. PubMed PMID: 21697253.61.
Russell, A. B., Trapnell, C., and Bloom, J. D. (2018). Extreme heterogeneity of influenza virus infection in single cells. Elife 7.
Sassi, F., Benazzi, C., Castellani, G., and Sarli, G. (2010). Molecular-based tumour subtypes of canine mammary carcinomas assessed by immunohistochemistry. BMC Vet Res 6, 5.
Savary G, Dewaeles E, Diazzi S, Buscot M, Nottet N, Fassy J, et al. The Long Noncoding RNA DNM3OS Is a Reservoir of FibromiRs with Major Functions in Lung Fibroblast Response to TGF-beta and Pulmonary Fibrosis. Am J Respir Crit Care Med. 2019; 200(2):184-98. Epub 2019/04/10. doi: 10.1164/rccm.201807-1237OC. PubMed PMID: 30964696.64.
Sivan, G., Martin, S. E., Myers, T. G., Buehler, E., Szymczyk, K. H., Ormanoglu, P., and Moss, B. (2013). Human genome-wide RNAi screen reveals a role for nuclear pore proteins in poxvirus morphogenesis. Proc Natl Acad Sci USA 110, 3519-3524.
Sivan, G., Ormanoglu, P., Buehler, E. C., Martin, S. E., and Moss, B. (2015). Identification of Restriction Factors by Human Genome-Wide RNA Interference Screening of Viral Host Range Mutants Exemplified by Discovery of SAMD9 and WDR6 as Inhibitors of the Vaccinia Virus K1L-C7L-Mutant. MBio 6, e01122.
Soares, J. A., Leite, F. G., Andrade, L. G., Tones, A. A., De Sousa, L. P., Barcelos, L. S., Teixeira, M. M., Ferreira, P. C., Kroon, E. G., Souto-Padron, T., et al. (2009). Activation of the PI3K/Akt pathway early during vaccinia and cowpox virus infections is required for both host survival and viral replication. J Virol 83, 6883-6899.
Steuerman, Y., Cohen, M., Peshes-Yaloz, N., Valadarsky, L., Cohn, O., David, E., Frishberg, A., Mayo, L., Bacharach, E., Amit, I., et al. (2018). Dissection of Influenza Infection In Vivo by Single-Cell RNA Sequencing. Cell Syst 6, 679-691 e674.
Tai C J, Liu C H, Pan Y C, Wong S H, Richardson C D, Lin L T. (2019 May). Chemovirotherapeutic treatment using Camptothecin enhances oncolytic measles virus mediated killing of breast cancer cells. Sci Rep.; 9(1):6767. doi: 10.1038/s41598-019-43047-3.
Thorne, S. H. (2011). Immunotherapeutic potential of oncolytic vaccinia virus. Immunol Res 50, 286-293.
Vassaux G, Hurst H C, Lemoine N R. Insulation of a conditionally expressed transgene in an adenoviral vector. Gene Ther. 1999; 6(6):1192-7. Epub 1999/08/24. doi: 10.1038/sj.gt.3300910. PubMed PMID: 10455425.60.
Vassaux G, Zwarthoed C, Signetti L, Guglielmi J, Compin C, Guigonis J M, et al. Iodinated Contrast Agents Perturb Iodide Uptake by the Thyroid Independently of Free Iodide. J Nucl Med. 2018; 59(1):121-6. Epub 2017/10/21. doi: 10.2967/jnumed.117.195685 jnumed.117.195685 [pii]. PubMed PMID: 29051343.65.
Walsh, D., Arias, C., Perez, C., Halladin, D., Escandon, M., Ueda, T., Watanabe-Fukunaga, R., Fukunaga, R., and Mohr, I. (2008). Eukaryotic translation initiation factor 4F architectural alterations accompany translation initiation factor redistribution in poxvirus-infected cells. Mol Cell Biol 28, 2648-2658.
Williams, J. A. (2018). Using PDX for Preclinical Cancer Drug Discovery: The Evolving Field. J Clin Med 7.
Yang, Z., Bruno, D. P., Martens, C. A., Porcella, S. F., and Moss, B. (2010). Simultaneous high-resolution analysis of vaccinia virus and host cell transcriptomes by deep RNA sequencing. Proc Natl Acad Sci USA 107, 11513-11518.
Zhang, Q., Yu, Y. A., Wang, E., Chen, N., Danner, R. L., Munson, P. J., Marincola, F. M., and Szalay, A. A. (2007). Eradication of solid human breast tumors in nude mice with an intravenously injected light-emitting oncolytic vaccinia virus. Cancer Res 67, 10038-10046.

1-18. (canceled)

19. A method of assessing the sensitivity or resistance of a subject having a cancer to an oncolytic virus, which method comprises a step of determining, in a biological sample from said subject selected from the group consisting of a tumor sample, a blood sample, a serum sample, a plasma sample and a derivative thereof, the presence or absence of a protein/mRNA encoded by a gene selected from the group consisting of DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, thereby assessing whether the subject having a cancer is sensitive or resistant to the oncolytic virus.

20. The method according to claim 19, wherein the method comprises
a step a) of determining, in a biological sample from said subject selected from the group consisting of a tumor sample, a blood sample, a serum sample, a plasma sample and a derivative thereof, the presence or absence of, and if present the expression level of, and/or percentage of cells expressing, a protein/mRNA encoded by a gene selected from the group consisting of DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, and,
when the expression level of, and/or percentage of cells expressing, the protein/mRNA is determined, a step b) of comparing said expression level to a reference expression level and/or said percentage of cells to a reference percentage of cells, thereby assessing whether the subject having a cancer is sensitive or resistant to the oncolytic virus.

21. The method according to claim 20, wherein
the protein/mRNA expression level determined in step a) is the protein/mRNA basal expression level in the subject, and the percentage of cells expressing the protein determined in step a) is the basal percentage of cells expressing the protein in the subject, and wherein
step b) comprises comparing said protein/mRNA basal expression level to a protein/mRNA response expression level in the subject as determined after an administration to said subject of the oncolytic virus, and/or comparing said basal percentage of cells to a percentage of cells expressing the protein in the subject as determined after an administration to said subject of the oncolytic virus.

22. The method according to claim 20, wherein the protein/mRNA basal expression level and/or basal percentage of cells expressing the protein, is determined before any step of cancer treatment applied to the subject or at about the same time as beginning a cancer treatment.

23. The method according to claim 19, wherein the cancer is selected from the group consisting of a carcinoma, a sarcoma, a lymphoma, a melanoma, a paediatric tumour and a leukaemia.

24. The method according to claim 23, wherein the cancer is selected from the group consisting of a breast cancer, a breast cancer comprising triple negative carcinoma cells (TNBC), a colon cancer, a skin cancer, a melanoma, a lung cancer, a glioblastoma multiform, an osteosarcoma, a soft tissue sarcoma, an ovarian cancer, a prostate cancer, a lymphoma, and an acute myeloid leukemia.

25. The method according to claim 20, wherein the cancer is a breast cancer and a DDIT4 protein/mRNA basal expression level above a protein/mRNA reference expression level, or a percentage of cells expressing a DDIT4 protein above a reference percentage of cells, is indicative of a resistance of the subject to the oncolytic virus, and a DDIT4 protein/mRNA basal expression level below said protein/mRNA reference expression level, or a percentage of cells expressing a DDIT4 protein below said reference percentage of cells, is indicative of a sensitivity of the subject to the oncolytic virus.

26. The method according to claim 19, wherein the virus is a vaccinia virus or an attenuated version thereof.

27. The method according to claim 26, wherein the vaccinia virus is selected from the group consisting of a Lister, a Copenhagen and a Western Reserve strain.

28. A method of selecting a treatment comprising an oncolytic virus effective against a cancer of a subject, wherein the method comprises, in the following order:
a step a) of determining the presence or absence of the basal expression level of, or basal percentage of cells expressing, a protein/mRNA encoded by a gene selected from the group consisting of DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in a biological sample of a subject having a cancer, before any step of cancer treatment comprising an oncolytic virus applied to the subject or at about the same time as beginning a cancer treatment comprising an oncolytic virus in the subject,
a step a′) of determining, in a biological sample of the subject having a cancer, the protein/mRNA response expression level, or percentage of cells expressing the protein, after the administration to said subject of at least one therapeutic dose of an oncolytic virus for treating the cancer,
a step b) of:
comparing said protein/mRNA response expression level to said protein/mRNA basal expression level and/or to a protein/mRNA reference expression level in a control population, and/or of comparing said percentage of cells expressing the protein to said basal percentage of cells and/or to a reference percentage of cells in a control population, and
a step c) of selecting an appropriate treatment of the subject's cancer, wherein:
a protein/mRNA response expression level identical to or below the protein/mRNA basal and/or reference expression level, and/or a percentage of cells expressing the protein identical to or below the basal and/or reference percentage of cells, is the indication that the oncolytic virus will be effective as such and is to be selected for treating the cancer of the subject, whereas a protein/mRNA response expression level above the protein/mRNA basal and/or reference expression level, and/or a percentage of cells expressing the protein above the basal and/or reference percentage of cells, is the indication that the oncolytic virus will not be effective as such, and that an appropriate treatment is to be selected, the appropriate treatment being a treatment combining said oncolytic virus with an additional compound selected from the group consisting of DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, or a treatment comprising a therapeutic recombinant oncolytic virus.

29. A method of monitoring in a subject the response to a cancer treatment comprising an oncolytic virus, and if required of stopping or adapting the treatment, the method comprising
a step a) of determining at a first time point, T0, the expression level of (reference expression level), and/or the percentage of cells expressing (reference percentage of cells), a protein/a mRNA encoded by a gene selected from the group consisting of DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in a biological sample of a subject having a cancer, before any step of cancer treatment applied to the subject or at about the same time as beginning a cancer treatment in the subject, or after a step of cancer treatment applied to the subject,
a step a′) of determining in a biological sample of the subject having a cancer obtained at a different time point following T0 (T1), the protein/mRNA response expression level and/or percentage of responding cells expressing the protein, after the administration to said subject of a first, or additional, therapeutic dose of an oncolytic virus for treating the cancer, and
a step b) of comparing said protein/mRNA response expression level to said protein/mRNA reference expression level and/or to a protein/mRNA reference expression level in a control population, and/or of comparing said percentage of responding cells expressing the protein to said reference percentage of cells and/or to a reference percentage of cells in a control population, a protein/mRNA response expression level identical to or below the protein/mRNA reference expression level(s), and/or a percentage of responding cells expressing the protein identical to or below the reference percentage of cells, being the indication that the oncolytic virus will be effective as such against the cancer of the subject, whereas a protein/mRNA response expression level above the protein/mRNA reference expression level(s), and/or a percentage of responding cells expressing the protein above the reference percentage of cells, being the indication that an oncolytic virus will not be effective alone in the subject, and if the oncolytic virus is not effective as such, and
a step c) of stopping or adapting the treatment of the subject's cancer by selecting a treatment combining said oncolytic virus with an additional compound selected from the group consisting of DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, or a treatment comprising a therapeutic recombinant oncolytic virus.

30. A therapeutic recombinant virus comprising a nucleic acid for modulating a gene selected from the group consisting of DDIT4, SERPINE1, BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB or its expression product in a cell, the nucleic acid being selected from the group consisting of a si-RNA, a sh-RNA, an antisense-DNA, an antisense-RNA and a ribozyme when the modulation is an inhibition.

31. The therapeutic recombinant virus according to claim 30, wherein the therapeutic recombinant virus is a therapeutic recombinant oncolytic virus.

32. A pharmaceutical composition comprising a therapeutic recombinant virus according to claim 30 and pharmaceutically acceptable carrier(s) and/or excipient(s).

33. A kit comprising detection means selected from the group consisting of at least one antibody specific to DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 or TBCB; a molecule allowing the antibody detection; and, optionally, a leaflet providing the DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and/or TBCB respective reference expression level(s), and/or reference percentages of cells expressing a protein selected from the group consisting of DDIT4, SERPINE1 BHLHE40, HAS2, MT2A, AMOTL2, PTRF, SLC20A1, ZYX, CDKN1A, CYP1B1, LIF, NEDD9, NUAK1, PLAU, THBS1, DUSP6, APEX1 and TBCB, in control population(s), and/or a therapeutic recombinant virus.
